(19) United States
(12) Patent Application Publication
(10) Pub. No.: US 2023/0031222 A1
(43) Pub. Date: Feb. 2, 2023

(54) Systems and Methods for Processing Cells

(71) Applicant: Synthego Corporation, Redwood City, CA (US)

(72) Inventors: Myo Thu MAUNG, Pacifica, CA (US); Matthew Everly FOWLER, Oakland, CA (US); Paul DABROWSKI, Redwood City, CA (US); Sergey SHKAPOV, Belmont, CA (US); Ivan RAZINKOV, San Jose, CA (US); Jingling LI, Sunnyvale, CA (US); Daniel SLOMSKI, Atherton, CA (US); Jeffrey SMITH, Redwood City, CA (US); James Duncan BRAZA, East Palo Alto, CA (US); Aliya KUSUMO, Redwood City, CA (US); Brandon Phillip WHITNEY, San Francisco, CA (US)

(21) Appl. No.: 17/951,680

(22) Filed: Sep. 23, 2022

Related U.S. Application Data
(63) Continuation of application No. PCT/US2022/012820, filed on Jan. 18, 2022.
(60) Provisional application No. 63/138,197, filed on Jan. 15, 2021.
(60) Provisional application No. 63/225,383, filed on Jul. 23, 2021.

(51) Int. Cl. C12M 3/00; C12M 1/00

(52) U.S. Cl. CPC C12M 23/44; C12M 29/18

(57) ABSTRACT

A system for processing cells is provided. The system can include a cell culture container, a fluid handling device, and one or more removable cell processing modules for performing one or more cell processing processes. The one or more removable cell processing modules can include a fluid handling pathway. The one or more removable cell processing modules can be fluidly connected to the cell culture container and the fluid handling device via a receptacle in which the cell processing modules may be inserted. The system can be a closed system.

### CROSS-REFERENCE TO RELATED APPLICATIONS

This application is a continuation application of International Application No. PCT/US2022/012820, filed Jan. 18, 2022, which claims priority to U.S. Provisional Application No. 63/138,197 filed Jan. 15, 2021, entitled “Systems and Methods for Processing Cells,” and to U.S. Provisional Application No. 63/225,383 filed Jul. 23, 2021, entitled “Systems and Methods for Processing Cells,” each of which is hereby incorporated by reference in its entirety.

### BACKGROUND

Ex-vivo cell culturing allows cells to be grown externally in a nutrient rich solution, which can be used for many applications including experiments on certain cell types, production of biological products (e.g., those produced by the cells), production of cells to be used to treat certain diseases, etc. While cell culturing has numerous applications, it is often difficult to ensure that the cells maintain their viability throughout the cell culturing duration. Additionally, it can be difficult ensuring that particular batches of cells from a culture receive their intended processing treatment (e.g., purification). Thus, it would be desirable to have improved systems and methods for processing cells.

### SUMMARY OF THE DISCLOSURE

Some embodiments of the disclosure provide a system for processing cells. The system can include a cell culture container, a fluid handling device, and one or more removable cell processing modules for performing one or more cell processing processes. The one or more removable cell processing modules can include a fluid handling pathway. The one or more removable cell processing modules can be fluidly connected to the cell culture container and the fluid handling device. The system can be a closed system.

Some embodiments of the disclosure provide a method of processing cells. The method can include growing or incubating cells in a cell culture container and flowing the cells and/or one or more reagents through one or more removable cell processing module and performing a cell processing process in the one or more removable cell processing module, the one or more removable cell processing module can include a fluid handling pathway. The method can include a fluid handling device for handling fluids. The one or more removable cell processing modules can be connected to the cell culture container and the fluid handling device. The processing of cells can be carried out in a closed system.

Some embodiments of the disclosure provide a method for storing cells in a bag-based cell storage container. The method can include storing cells in one or more fluoropolymer membrane chambers of the bag-based cell storage container. The one or more fluoropolymer membrane chambers can include a non-fluoropolymer base.

Some embodiments of the disclosure provide a self-sterilizing connection. The self-sterilizing connection can include a sterile inner cavity, a sterile first barrier sealing the inner cavity, and a sterile needle in the inner cavity. The needle can include an inner channel. The self-sterilizing connection can include a second barrier sealing a sterile inner lumen. The inner cavity, the first barrier, and the needle can be part of a first device. The second barrier and the inner lumen can be part of a second device. The second barrier can be exposed to a sterilization agent. The second barrier can be aligned with the first barrier and an actuation force can be applied to drive the needle of the first device through both barriers to make a sterile connection with the inner lumen of the second device.

Some embodiments of the disclosure provide a method of making a sterile connection between a first device and a second device. The method can include providing a self-sterilizing connection. The self-sterilizing connection can include a sterile inner cavity, a sterile first barrier sealing the inner cavity, a sterile needle in the inner cavity, the needle can include an inner channel, and a second barrier that can seal a sterile inner lumen. The inner cavity, the first barrier, and the needle can be part of the first device, and the second barrier and the inner lumen can be part of the second device. The method can include exposing the second barrier to a sterilization agent, aligning the second barrier with the first barrier, and applying an actuation force to drive the needle of the first device through both barriers to make a sterile connection with the inner lumen of the second device.

Some embodiments of the disclosure provide a cell processing system. The cell processing system can include a cell culture container having an interior volume configured to receive cells, a receptacle having a flow coupler with a flow path, the flow coupler being actuatable to place the flow path of the flow coupler in fluid communication with the interior volume of the cell culture container, a cell processing module defining a second flow path that is in fluid communication with the flow path of the flow coupler, the cell processing module being configured to perform one or more cell processes as cells from the interior volume of the cell culture container flow along the second flow path. The receptacle can draw fluid from the cell culture container, through the flow path of the flow coupler, and through the second flow path of the cell processing module. The flow paths can be sealed and fluidically isolated from the ambient environment surrounding the cell culture container.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The following drawings are provided to help illustrate various features of embodiments of the disclosure and are not intended to limit the scope of the disclosure or exclude alternative configurations.

FIG. 1 shows a block diagram providing a schematic illustration of a cell processing system.

FIG. 2 shows an isometric view of a cell culture container.

FIG. 3 shows an isometric view of another cell culture container.

FIG. 4 shows a cross-sectional view of another cell culture container in closed configuration.

FIG. 5 show a cross-sectional view of the cell culture container of FIG. 4, in an open configuration.

FIG. 6 shows a schematic illustration of an example of a cell culture container engaged with a receptacle.

FIG. 7 shows a schematic illustration of an example of a cell culture container engaged with a simplified receptacle.

FIG. 8 shows a front cross-sectional view of a centrifuge container.

FIG. 9 shows an exploded view of the centrifuge container of FIG. 8.

FIG. 10 shows a top isometric view of a fluid handling device for receiving a cell culture container.

FIG. 11 shows a bottom isometric view of the fluid handling device of FIG. 10.

FIG. 12 shows another perspective view of the fluid handling device of FIG. 10, with portions of the fluid handling device opened for visual clarity.

FIG. 13 shows a cross-sectional view of the fluid handling device of FIG. 10 engaged with the cell culture container of FIG. 2 and with the flow coupler of the fluid handling device of FIG. 10 deployed.

FIG. 14 shows an enlarged cross-sectional view of FIG. 13 that details the engagement between the flow coupler and the cell culture container of FIG. 13.

FIG. 15 shows a rear perspective view of the fluid handling device of FIG. 10 with different cell processing modules.

FIG. 16 shows a front isometric view of a fluid handling device engaged with a cell culture container, and a cell processing module.

FIG. 17 shows a partial side view of the fluid handling device of FIG. 16 with the moveable rack positioned in an open configuration.

FIG. 18 also shows a partial side view of the fluid handling device of FIG. 16 with the moveable rack in an open configuration, and with the cell processing module and the cell culture container supported by the moveable rack.

FIG. 19 show a partial side view of the fluid handling device of FIG. 16 with the moveable rack in a closed configuration.

FIG. 20 shows a rear perspective view of the fluid handling device of FIG. 16 with the moveable rack in the closed configuration.

FIG. 21 shows a partial rear isometric view of a top plate of the fluid handling device of FIG. 16.

FIG. 22 shows a side cross-sectional view of the fluid handling device of FIG. 16.

FIG. 23 shows an enlarged partial top perspective view of the fluid handling device of FIG. 16.

FIG. 24 shows a schematic illustration of a cell processing system.

FIGS. 25 and 26 collectively show a flowchart of a process for performing a cell debeading process.

FIG. 27 shows a schematic illustration of another cell processing system.

FIGS. 28 and 29 collectively show a flowchart of a process for adding, removing and/or exchanging one or more reagents.

FIG. 30 shows a schematic illustration of another cell processing.

FIGS. 31 and 32 collectively show a flowchart of a process for performing a cell isolation process.

FIG. 33 shows a front perspective view of a cell processing module dispenser.

FIG. 34 shows a front perspective view of a cell processing system that includes a fluid handling device.

FIG. 35 shows a front view of the fluid handling device of the cell processing system of FIG. 34.

FIG. 36 shows a front isometric view of a plurality of cell processing systems and other instruments.

FIG. 37 shows an isometric view of the sampling instrument of FIG. 36.

FIG. 38 shows an isometric view of another sampling instrument.

FIG. 39 shows a front isometric view of another cell culture container.

FIG. 40 shows a bottom view of the cell culture container of FIG. 39.

FIG. 41 shows a cross-sectional view of the cell culture container of FIG. 39.

FIG. 42 shows another cross-sectional view of the cell culture container of FIG. 39.

FIG. 43 shows an isometric view of a mixer system.

FIG. 44 shows a front view of a gripper assembly of the mixer system of FIG. 43.

FIG. 45 shows the grippers of the gripper assembly of FIG. 44 positioned in the open configuration.

FIG. 46 shows an isometric view of the cell culture container of FIG. 45 received within the gripper, and with the gripper coupled to the rotor.

FIG. 47 shows a top view of the configuration of FIG. 46.

FIG. 48 shows a graph of the cell density divided by the true cell density as a percent for each mixing routine.

FIG. 49 shows an isometric front view of an electroporator module that is configured to electroporate cells from a cell culture container.

FIG. 50 shows an isometric rear view of the electroporator module of FIG. 49.

FIG. 51 shows a front isometric view of the electrode and the spacer of the electroporator module of FIG. 49, with an electrode removed for visual clarity.

FIG. 52 shows a side view of the electrode and the spacer of FIG. 51.

FIG. 53 shows an isometric view of another electroporator module.

FIG. 54 shows an isometric view of the spacer of the electroporator module of FIG. 53.

FIG. 55 shows a front view of the spacer of FIG. 54.

FIG. 56 shows a schematic illustration of another cell processing system.

FIG. 57 shows an isometric view of another cell processing module.

FIG. 58 shows a bottom view of the cell processing module of FIG. 57.

FIG. 59 shows a top view of the cell processing module of FIG. 57.

FIG. 60 shows a front view of the cell processing module.

FIG. 61 shows a schematic illustration of a flow coupler prior to engagement with a cell culture container.

FIG. 62 shows a schematic illustration of the flow coupler of FIG. 61 engaged with the cell culture container of FIG. 61.

FIG. 63 shows a schematic illustration of another fluid handling device prior to engagement with another cell processing module.

FIG. 64 shows an isometric view of another cell processing module.

FIG. 65 shows a schematic illustration of the cell processing module of FIG. 64, showing the interfacing with pressure sources of a fluid handling device.

FIGS. 66A and 66B collectively show a flowchart of a process 1500 for processing cells.

FIG. 67 shows a graph comparing the total viable cells and density of cells for the CARE system as well a standard flask.

FIG. 68 shows a graph comparing a TRAC gene knock-out scores in CD4+ Primary Human T cells using a CARE electroporator vs Lonza’s 4D-Nucleofector electroporation system.

FIG. 69 shows a graph of ddPCR data for TRAC gene editing in CD4+ Human primary T cells for two fresh runs.

FIG. 70 shows another graph of ddPCR data for TRAC gene editing in CD4+ Human primary T cells for two thawed runs.

FIG. 71 shows a graph of the performance of the CARE automated hardware for magnetic isolation of CD4+ T cells from fresh and thawed (from frozen) human PBMCs.

FIG. 72 shows a graph of the fold expansion of Human CD4+ T cells processed on the CARE hardware platform under 3 different conditions.

FIG. 73 shows a graph of the viability of Human CD4+ T cells isolated and cultured in the CARE hardware and consumables.

FIG. 74 shows a graph of the viability as a percentage for two independent T cell donors.

FIG. 75 shows a graph of the cell expansion folds over a number of days for the two independent T-cell donors.

FIG. 76 shows a graph of the total number of viable cells over the number of days for the two independent T-cell donors.

### DETAILED DESCRIPTION OF THE PRESENT DISCLOSURE

As described above, it is often difficult to ensure that the cells maintain their viability throughout the culturing process (e.g., ensuring that the cells do not die during culturing). For example, cells are typically cultured in flasks (e.g., Erlenmeyer flasks) that are sealed with metal foils, or sealable plastic sheets. While these may help to prevent the culture from being exposed to the ambient environment (that can directly kill cells or otherwise force nutrient levels such as oxygen within the culturing solution out of balance), these are only temporary solutions that provide only mediocre sealing from the ambient environment. In fact, the generation of the seal from the ambient environment is largely operator dependent, and thus can be highly variable between operators. Additionally, and regardless of the operator, the temporary nature of these seals can, in routine practice, expose the cells to the ambient environment. For example, if the cells are to be processed in any manner, such as concentrated (e.g., to move the cells to a larger container), this requires exposure and transport of the cells to the cell concentrator instrumentation, and back to another container. This is only an example of a single cell processing step, which is typically uncharacteristic. Rather, multiple cell processing steps are usually implemented, each of which thus exposes the cells to the ambient environment.

Some embodiments of the disclosure provide advantages to these issues (and others) by providing improved systems and methods for processing cells. For example, some embodiments of the disclosure provide a closed cell processing system that can function in an isolated manner from the ambient environment, thereby decreasing the risks to over exposure to the ambient environment, e.g., contamination, that may detrimentally impact cell viability. In some embodiments, the cell processing system can include a number of cell processing modules that can be in selective fluid communication with the container that the cells are being cultured in. These cell processing modules can each implement a particular cell culturing functionally including magnetic separation, transfection, media exchange, etc. When in use, a particular cell processing module is brought into fluid communication with the container that houses the cells. Because the cell processing module is isolated from the ambient environment, as the cells from the container flow through the particular cell processing module, the cells are not exposed to the ambient environment. In this way, a number of cell processes can be imposed on the cells in such an end-to-end cell processing system, while being isolated from contact with the ambient embodiment. Still further, because of the flexibility of the cell processing modules, many different cell processes can be imposed on the cells without having to move the cells to a different instrument. In addition, the ability to physically disconnect individual cell processing steps via use of various cell processing modules configured to perform each cell processing step allows maximization of the utility of the individual hardware components and of the physical space (e.g., cleanroom, bedside or benchtop space) occupied by the cell processing system. In some configurations, this ease of switching cell processes, allows for a more automated cell culturing system, which can free current operator constraints for cell culturing (e.g., a given operator can be much more efficient). In some embodiments, a plurality of cell processing systems described herein operate in parallel to carry out a plurality of cell processes simultaneously thereby allowing production of different cell therapies in parallel in a high throughput fashion. In certain embodiments, each cell processing system of the of cell processing systems operating in parallel is capable of being inactivated without interrupting the cell manufacturing process.

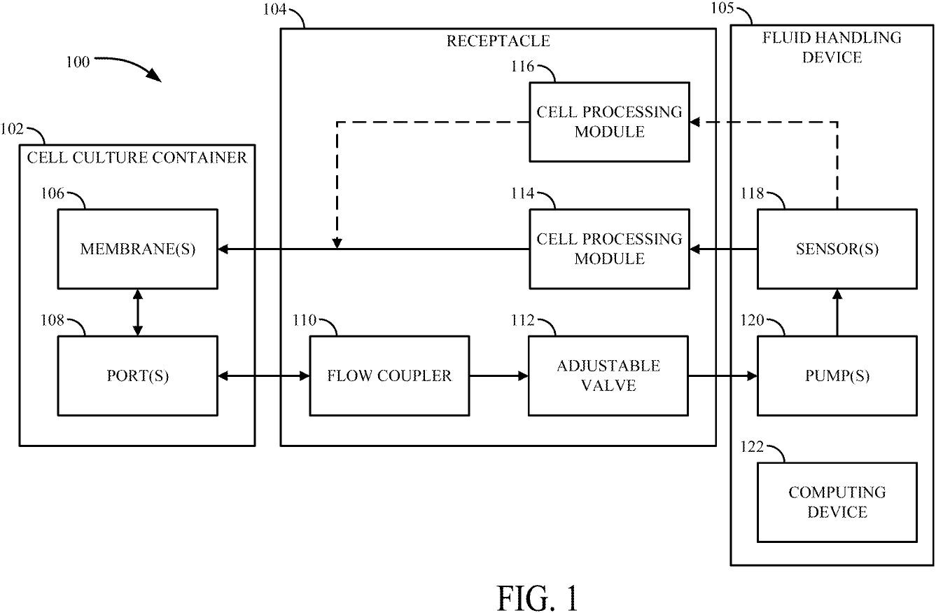

FIG. 1 is a schematic illustration of one embodiment of a cell processing system 100 in accordance with the present disclosure. The cell processing system 100 can include a cell culture container 102, one or more cell processing modules 114,116, and a fluid handling device 105. The cell processing system also may include a receptacle 104 for receiving the cell culture container 102 and the one or more cell processing modules 114,116. The cell culture container 102 can be engaged (and disengaged) with the receptacle 104 to secure (remove) the cell culture container 102 relative to the receptacle 104. For example, the cell culture container 102 can be slideably engaged and disengaged with the receptacle 104 and/or aligned with the receptacle 104 via pins. The receptacle 104 can be engaged in fluid communication with flow paths of the fluid handling device 105 (e.g., the fluid handling device 105 can exert pressure on the receptacle 104 via flow paths, however, no cells, media or other reagents comprised in the receptacle 104 is communicated to the fluid handling device 105), and can be selectively placed into and out of fluid communication with the cell culture container 102 (e.g., cells, media or other reagents comprised in the cell culture container 102 can be communicated between the receptacle 104 and the cell culture container 102). The cell processing system also may be configured to engage the cell culture container 102 directly with the one or more cell processing modules 114,116. For example, the cell culture container 102 can be engaged and disengaged with the with the one or more cell processing modules 114, 116 via a self-sterilizing connection. The one or more cell processing modules 114, 116 can be engaged in fluid communication with flow paths of the fluid handling device 105 (e.g., the fluid handling device 105 can exert pressure on the cell processing modules 114, 116 via flow paths, however, no cells, media or other reagents comprised in the cell processing modules 114, 116 is communicated to the fluid handling device 105), and can be selectively placed into and out of fluid communication with the cell culture container 102 (e.g., cells, media or other reagents comprised in the cell culture container 102 can be communicated between the cell processing modules 114, 116 and the cell culture container 102).

As shown, the cell culture container 102 can include a membrane 106 and a port 108. The membrane 106 can define an interior volume of the cell culture container 102 that receives cell culture fluid medium and cells. In some configurations, the membrane 106 can be a single, integral piece, in the form of a bag to encapsulate and define the interior volume. In other cases, the membrane 106 can be multiple pieces that are joined to define the interior volume of the cell culture container 102. For example, the membrane 106 can define two planar halves that can be mechanically secured at joined peripheral edges to define the interior volume. In some embodiments, the cell culture container 102 can include a frame that houses or sandwiches the membrane 106 or membranes 106. For example, the frame can extend around the periphery of the membrane 106 or membranes on the top side and/or bottom side of the membrane 106 or membranes 106 and sandwich the membrane 106 or membranes 106. In some cases, a top portion of a frame can be joined together mechanically, via fasteners (e.g., bolts) to a bottom portion of the frame, such that the membrane 106 or membranes 106 are housed and/or sandwiched between the two portions of the frame. In some configurations, such as when the membrane 106 is implemented as two pieces, fastening of the frame can sandwich and clamp together the opposing pieces of the membrane 106 along the periphery of both pieces of the membrane 106.

The cell culture container 102 can include a single port 108, or multiple ports 108. In some cases, a port 108 can be disposed on one side (e.g., an upper side) of the cell culture container 102 to allow access to the interior volume of the cell culture container 102. For example, the port 108 can include a septum that is secured to (or integrated within) the membrane 106 (or frame) of the cell culture container 102. The septum may have a lower surface that provides a seal with the internal volume of the cell culture container 102, isolating the internal volume from the ambient environment. The port 108 also may have a bore positioned above the septum that defines the port 108, which can provide a fluid path from the internal volume of the cell culture container 102, when the lower surface of the septum is pierced. In some cases, the port 108 can be a valve that can be actuated between a closed position that isolates the internal volume from the ambient environment, to an open position that allows fluid communication from the interior volume and along a flow path that opens the valve.

In some cases, the cell culture contain may include a second port 108, which optionally may be positioned on another side of the cell culture container 102 than the first port 108, or the second port 108 may be position on the edge of the cell culture container 102. The second port 108 also provide fluid communication between the interior volume of the cell culture container 102 and another flow path, such as when the membrane 106 is pierced at the second port 108, for example, through a septum located at the second port 108. In some cases, the second port may comprise or interact with a septum and/or a valve, which may be utilized to engage the interior of the cell culture container. In some configurations, the cell culture container 102 can have an alignment feature that aligns and engages with a corresponding alignment feature of the receptacle or a cell processing module 114, 116 to generate a proper alignment between the cell culture container 102 and the receptacle 104 or the cell processing module 114, 116.

As shown, the receptacle 104 or the cell processing module 114, 116 can include a flow coupler 110 for engaging the interior of the cell culture container 102. The flow coupler 110 can be actuated to engage with the interior of the cell culture container 102 through the port 108 of the cell culture container 102 to bring the flow path of the receptacle or the cell processing module into fluid communication with the interior volume of the cell culture container 102. The flow coupler 110 may include a reciprocating component with a flow path directed therethrough, so that when the reciprocating component engages with the port 108, the flow path of the reciprocating component is brought into fluid communication with the interior volume of the cell culture container 102. The flow coupler 110 can be utilized in different ways. For example, the flow coupler 110 can include a needle that is biased (e.g., with a spring) towards a first position that is not in contact with the cell culture container 102. The needle can be then be actuated to a second position (e.g., a stopper can move out of contact with the reciprocating component to release the biasing force) to advance the needle through the port 108 and into the interior volume of the cell culture container 102. In other configurations, such as when the port 108 comprises and/or interacts with a valve, the reciprocating member (e.g., a reciprocating cylinder) can be advanced to contact the valve, move the valve from a closed position to an open position, and allow fluid communication between the flow path of the reciprocating component and the interior volume of the cell culture container 102.

In some embodiments, the receptacle 104 may include one or more adjustable valves 112 (e.g., a three way valve, a multi-way valve, a rotational valves such as rotary valves), that can be adjusted to selectively bring a particular cell processing module 114, 116 into or out of fluid communication with a flow path of the receptacle 104. The one or more adjustable valves 112 also may be adjusted to selectively bring the flow coupler 110 into or out of fluid communication with a flow path of the receptacle 104. In this way, fluid that includes cells of the cell culture container 102 can be passed through a selected cell processing module (e.g., the cell processing module 114). Then, as desired, the adjustable valve 112 can be adjusted to allow the fluid including the cells of the cell culture container 102 to flow through a different cell processing module (e.g., the cell processing module 116) and/or the adjustable valve 112 can be adjusted to allow the fluid including cells of the cell culture container to flow to the cell culture container 102. The one or more cell processing modules 114, 116 have the ability to implement a cell process on the cells as they flow through the particular cell processing module 114, 116. For example, a process for a cell processing module can include cell enrichment, cell isolation, electroporation, cell isolation, cell media exchange, etc. Thus, the types of components within a cell processing module 114, 116 may depend on the process the cell processing module is configured to perform. As such, a cell processing module 114,116 may include, reagent reservoirs, pumps, valves, circuitry (e.g., spaced apart electrodes), etc. Although only two cell processing modules 114,116 are illustrated in FIG. 1, in alternative configurations, the cell processing system 100 or the receptacle can include other numbers of cell processing modules (e.g., three, four, five, or more).

In some embodiments, the receptacle 104 or the cell processing module 114, 116 can include an alignment feature that engages with a corresponding alignment feature of the cell culture container 102. When the alignment feature of the receptacle 104 or the cell processing module 114, 116 engages with the alignment feature of the cell culture container 102, the port 108 of the cell culture container 102 is brought into alignment with the flow coupler 110. This ensures that the port 108 and the flow coupler 110 are in alignment prior to actuation of the flow coupler 110. The alignment features can be configured in different embodiments. For example, the alignment feature can be a bore (or a plurality of bores) and the corresponding alignment feature can be a protrusion (or a plurality of protrusions) that are received in the corresponding bore. As another example, the alignment feature can be a slot and the corresponding alignment feature can be a rail that slides along the slot. In this case, an end of the slot (or a mechanical stop) can ensure that one of the components (e.g., the cell culture container 102 or the receptacle 104 or the cell processing module 114, 116) does not advance past a particular location along the slot such that the port 108 of the cell culture container 102 is aligned with the flow coupler 110 of the receptacle 104 or of the cell processing module 114, 116.

In some embodiments, the cell processing modules 114, 116 can each have one or more alignment features that interface with corresponding alignment features on the receptacle 104, the fluid handling device 105, or both the receptacle 104 and the fluid handling device 105. For example, the receptacle 104 (or the fluid handling device 105) can have designated locations, which can be known to the computing device 122 (e.g., these locations indexed by the computing device 122). Each of these designated locations can have the one or more alignment features that can engage with the one or more alignment features of a cell processing module 114, 116. In this way, the cell processing modules 114, 116 (and others) can be easily inserted (or removed from engagement) with one of the locations in a cartridge-like manner (e.g., the cell processing modules 114, 116 being constructed as a cartridge). Additionally, the computing device 122 can know which designated locations having a cell processing module 114, 116 that is in use. In some embodiments, the cell processing modules 114, 116 can have sensors (e.g., proximity sensors, contact sensors, etc.), which can be used by the computing device 122 to determine that a particular cell processing module is interfaced with a particular designated location, and what type of cell processing module is interfaced with the particular designated location (e.g., a cell sorting cell processing module). Thus, the cell processing modules 114, 116 can have sensors that correspond to their unique cell processing function.

In some embodiments, the cell culture container 102 can be slidably engaged with the receptacle 104 or the cell processing module 114, 116. For example, the frame of the cell culture container 102 may be engaged with a channel of the receptacle 104 or the cell processing module 114, 116 and may slide along the channel of the receptacle 104 or the cell processing module 114, 116 until the cell culture container 102 is in proper alignment (e.g., in which the port 108 of the cell culture container 102 aligns with the flow coupler 110 of the receptacle 104 or the cell processing module 114, 116). In other cases, the cell culture container 102 can be releasably engaged with the receptacle 104 or the cell processing module 114, 116 (e.g., with pins). The releasable engagement (e.g., slideable engagement) between the cell culture container 102 and the receptacle 104 or the cell processing module 114, 116 ensures that the current cell culture container 102 is secured to the receptacle 104 or the cell processing module 114, 116 and can be removed from the receptacle 104 or the cell processing module 114, 116.

In some embodiments, the interior volume of the cell culture container 102 can be in a range of substantially (i.e., deviating by less than 10 percent from) 15 mL to substantially 750 mL, in a range of substantially 50 mL to substantially 700 mL, in a range of substantially 100 mL to substantially 600 mL, in a range of substantially 200 mL to substantially 500 mL, etc. In some cases, the interior volume of the cell culture container can be substantially 50 mL, substantially 200 mL, or substantially 500 mL

As shown, the fluid handling device 105 may include sensors 118, pumps 120, and a computing device 122 (or the fluid handling device may be operably connected to a computing device 122). The sensors 118 can include a sensor that is positioned along a flow path that receives fluid from the cell culture container 102 (e.g., through the receptacle 104 via the flow coupler 110 contained within the receptacle 104 and/or through the one or more cell processing modules 114,116 contained within the receptacle 104). In some cases, this sensor 118 can include a bubble sensor that determines a presence of air within the fluid path. In some configurations, the sensors 118 can include other types of sensors. The pump 120 is in fluid communication with a flow path and drives fluid through the receptacle 104 to the cell culture container 102, for example via the flow coupler 110 and/or via the flow coupler 110 contained within the receptacle 104 and/or through the one or more cell processing modules 114,116 contained within the receptacle 104. In some configurations, the pump 120 can be a syringe pump, a pneumatic pump, or a peristaltic pump.

In some embodiments, the computing device 122 is in communication with the electrical components of the fluid handling device 105 and the receptacle 104 or the cell processing module 114, 116. For example, the computing device 122 can be in communication with the sensors 118 and the pumps 120 of the fluid handling device 105, and the adjustable valve 112 of the receptacle 104 or the cell processing module 114, 116. In particular, the computing device 122 can cause the pumps 120 to pump fluid. The computing device 122 also may cause the valve 112 to adjust its position. The computing device 122 also may cause the one or more cell processing modules 114,116 to be actuated. The computing device 122 may be configured in different embodiments. For example, the computing device 122 may include one or more components such as a processor, memory, a display, inputs (e.g., a keyboard, a mouse, a graphical user interface, a touch-screen display, etc.), and communication devices. In some cases, the computing device 122 may comprise or consist of a processor. The computing device 122 may communicate directly or indirectly with other computing devices and systems. In some embodiments, the computing device 122 may actuate some or all of the cell processes disclosed herein.

In some embodiments, and as shown in FIG. 1, a flow path is defined or comprised by the cell processing system 100. For example, the cell culture container 102 may be engaged (e.g., slideably) with the receptacle 104 or the cell processing module 114, 116 and the port 108 of the cell culture container 102 may be aligned with the flow coupler 110 of the receptacle 104 or the cell processing module 114, 116. Then, the flow coupler 110 or a component of the flow coupler 110 (e.g., a reciprocating component) may be actuated such the flow coupler 110 or a portion or component of the flow coupler is inserted into the port 108 and a flow path is established between the interior volume of the cell culture container 102 and the receptacle 104 or the cell processing module 114, 116. Then, a computing device 122 may select which of the cell processing modules 114,116 is to be utilized by adjusting the valve position of the adjustable valve 112, so that the established flow path between the interior volume of the cell culture container 102 and the receptacle 104 or the cell processing module 114, 116 is directed through the cell processing module 114,116. In the illustrated embodiment, the computing device 122 has selected the cell processing module 114 to be utilized. The computing device 122 can cause the pump 120 to draw fluid (including cells) out of the cell culture container 102 through the port 108, through the receptacle or directly through the cell processing module 114 via the flow coupler 110, through the adjustable valve 112, and through a flow path of the cell processing module 114. As the fluid that includes the cells flows through the cell processing module 114, the cells are subjected to a process that is defined by the cell processing module 114 (i.e., a cell process that the cell processing module 114 is configured to perform). After the cell process is complete, the fluid that includes the cells can be returned to the cell culture container 102 (e.g., via the port 108). Because this flow path is isolated from the ambient environment (e.g., where the flow path is exposed to the ambient environment via 0.22 micron pore filter) the cells are isolated from the ambient environment. Additionally, the cell processing modules 114,116 may allow for cell processes to be automated -ensuring that the processes are completed automatically and no manual errors have occurred.

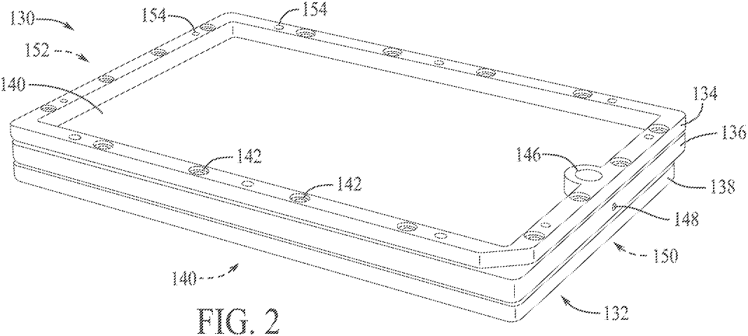

FIG. 2 shows an isometric view of one embodiment of a cell culture container 130, which is a specific configuration of the cell culture container 102 of FIG. 1. The cell culture container 130 can include a frame 132 having pieces 134, 136, 138, one or more membranes 140, frame fasteners 142, one or more ports 146,150, and alignment features 154. The frame 132 contains and secures the one or more membranes 140 within the frame 132. The frame 132 includes distinct pieces 134, 136, 138 that are joined together by the fasteners 142, and which extend around the periphery of the one or more membranes 140. As shown, the spacer piece 136 of the frame 132 is sandwiched between the upper pieces 134 and lower piece 138 of the frame 132 so that the upper piece 134 defines an upper side of the frame 132, while the lower piece 138 defines a lower side of the frame 132. The upper piece 134 and the lower piece 138 may have a centrally located interior opening so that when the upper piece 134 and the lower piece 138 are assembled with the one or more membranes 140, the one or more membranes 140 may expand and retract through the interior opening of the upper piece 134 of the frame 132 and/or through a corresponding interior opening of the lower piece 138 of the frame 132, e.g., wherein the cell culture container 130 includes two membranes 140 that form an interior volume. These interior openings of the upper piece 134 and the lower piece 138 allow the one or more membranes 140 to expand in order to increase the interior volume of the cell culture container 130 when the interior volume receives the cell culture media (and the cells).

The upper piece 134 and the lower piece 138 may be constructed in a similar manner and may include similar components and features. For example, the upper piece 134 of the frame 132 may have a protrusion that includes the port 146, while the lower piece 138 of the frame 132 also may include a protrusion that includes the port 150. Each port 146, 150 may include a bore that is directed through the respective entire thickness of the corresponding upper piece 134 and the lower piece 138 of the frame 132, and each port 146, 150 may include or engage a septum located on an end of the respective bore that isolates the interior volume of the membrane 140 from the ambient environment. As described herein, the septum may be pierceable to allow fluid communication through the bore, through the membrane 140, and into the interior volume of the cell culture container 130. In some configurations, the septum can be coupled to the surface of the respective portion of the frame 132, or can be integrated within the bore of the respective port.

Although not shown in FIG. 2, the spacer piece 136 of the frame 132 provides a barrier that separates the upper piece 134 and the lower piece 138 of the frame 132. The spacer piece 136 may include one or more ports 148, 152 located on opposing ends of the spacer piece 136. The spacer piece 136 can be constructed of any suitable material e.g., plastic such as polycarbonate etc. However, in alternative configurations, the ports 148, 152 can be located on the same side of the spacer piece 136. The port 148 may provide a flow pathway (e.g., a liquid flow pathway) through the spacer piece 136 to a surface of a membrane 140.

When assembled, peripheral edges of a membrane 140 are positioned between the upper piece 134 and the lower piece 138 (and optionally the spacer piece 136). Fasteners 142 (or other mechanical coupling configurations) may be used to couple together the pieces 134, 136, 138 of the frame 132. In some configurations, the ports 148, 150 can then be used to fill the respective membrane 140 with cell culture media (and cells). As shown, the piece 134 of the frame 132 includes alignment features in the form of bores 154 that are positioned at locations around the entire periphery of the spacer piece 136 of the frame 132. These bores 154 can be engaged with corresponding alignment features of the receptacle 104 or the cell processing module 114, 116 such as protrusions (or pins) that are inserted into respective bores 154. The engagement between an alignment feature of the cell culture container 130 (e.g., the bores 154) with an alignment feature of the receptacle 104 or the cell processing module 114, 116 aligns the port 146 with the flow coupler 110 of the receptacle 104 or the cell processing module 114, 116.

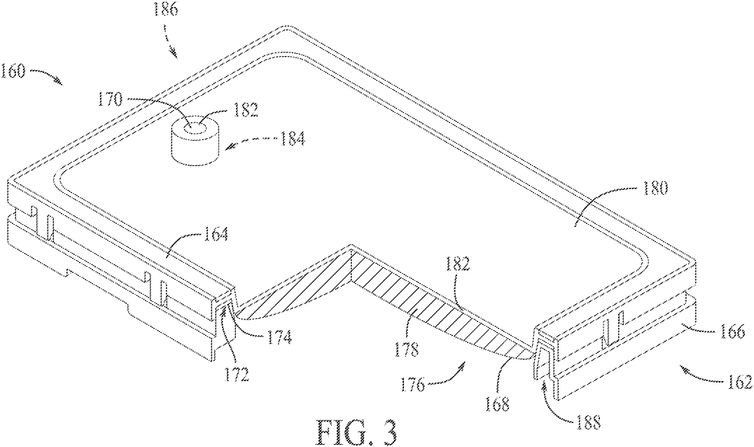

FIG. 3 shows an isometric view of another embodiment of a cell culture container 160, which is also a specific configuration of the cell culture container 102. The cell culture container 160 includes a frame 162 having an upper piece 164, a lower piece 166, a membrane 168, and a port 170. The frame 162 of the cell culture container 160 secures and houses the membrane 168. The upper piece 164 and the lower piece 166 of the frame 162 are coupled to secure the membrane 168 within the frame 162. In particular, the upper piece 164 of the frame 162 may have a peripheral channel 172 that extends along the entire periphery of the upper piece 164, while the lower piece 166 may have a peripheral protrusion 174 that extends along the entire periphery of the lower piece 166. The upper piece 164 and the lower piece 166 may be coupled via mating of the protrusion 174 and the channel 172. In some configurations, the upper piece 164 may have the peripheral protrusion 174 and the lower piece 166 may have the peripheral channel 172.

As shown, the lower piece 166 of the frame 162 has a centrally located interior opening 176, which is similar to the openings of the upper piece 134 and lower piece 138 of the frame 132. The interior opening 176 allows the membrane 168 to selectively expand and retract through the interior opening 176, allowing the interior volume 178 of the membrane 168, which includes the cell culture media and cells, to be modulated based on the volume of cell media and cells inserted in the cell culture container 160. The interior opening 176 may be located on either side of the cell culture container 160. As illustrated, the interior opening 176 is located an under side of the cell culture container 160. The upper piece 164 of the frame may have a surface 180 that extends entirely beyond the interior opening 176 of the lower piece 166, and provides a border that can partially define the interior volume 178. When the surface 180 is located on an upper side of the cell culture container 160, the membrane expands through the interior opening 176 of the lower piece 166.

Similarly to the cell culture container 130, the port 170 of the cell culture container 160 can include a bore 182, and a septum 184 (not shown) disposed at an end of the bore 182. The septum 184 provides a pierceable seal that separates the interior volume 178 of the cell culture container 160 from the ambient environment. In some cases, the septum 184 may be integrally formed with the upper piece 164 on the surface 180 of the upper piece 164. In some configurations, the septum 184 can be pierced (e.g., by the flow coupler 110 of the receptacle 104 or the cell processing module 114, 116) to allow fluid communication between the interior volume 178 and the component pierced by the septum 184 (e.g., by the flow coupler 110 of the receptacle 104 or the cell processing module 114, 116).

In some embodiments and similarly to the cell culture container 130, the cell culture container 160 can include an additional port 186 (not shown) that also provides access to the interior volume 178 of the cell culture container. In some cases, the additional port 186 may be configured in a similar manner as the port 170. In other cases, the additional port 186 can include a conduit directed through one or both of the pieces 164, 166 that is in fluid communication with the interior volume 178, and a check valve within the conduit (or in fluid communication with the conduit) that only allows fluid to flow through the conduit and into the interior volume 178.

In some embodiments, the cell culture container 160 can include an alignment feature 188 that can engage with a corresponding alignment feature of the receptacle 104 or the cell processing module 114, 116. As shown in FIG. 3, the alignment feature 188 is a channel that extends along the bottom peripheral edge of the lower piece 166 of the frame 162. In this case, the alignment feature of the receptacle 104 or the cell processing module 114, 116 can be a protrusion that extends in a similar manner as the channel of the cell culture container 160. This protrusion of the receptacle 104 or the cell processing module 114, 116 can be seated within the alignment feature 188 to couple the cell culture container 160 to the receptacle 104 or the cell processing module 114, 116 in a removably coupled manner.

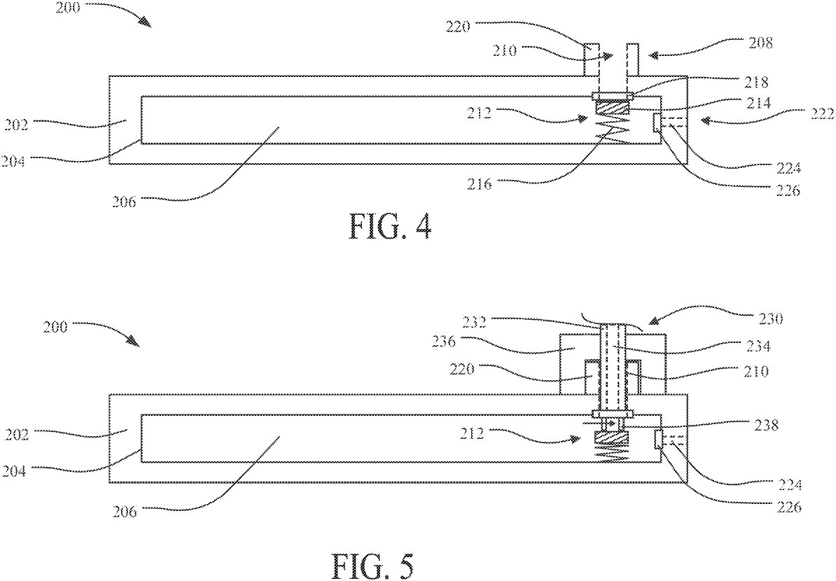

FIG. 4 and FIG. 5 show cross-sectional views other embodiments of a cell culture container 200, which are specific configurations of the cell culture container 102. Similarly to the other cell culture containers, the cell culture container 200 includes a frame 202, a membrane 204 that defines an interior volume 206 of the cell culture container 200 (which includes the cell culture media), and a port 208. The port 208 includes a bore 210 and a valve 212 having a seal 214 biased by a spring 216 against a valve seat 218. The bore 210 extends through an extension 220 that extends upwardly from the frame 202, through the frame 202, and through the valve seat 218. The valve seat 218 can be coupled to (or integrated within) the membrane 204, and the seal 214 seats against the valve seat 218 to generate a seal between the ambient environment that includes the bore 210. In this way, when the cell culture container 160 is not being used to process the cells (e.g., when the cells are growing), the valve 212 prevents fluid communication between the interior volume 206 and the ambient environment. Although the valve is illustrated as mainly residing within the interior volume 206 of the cell culture container 200, in other configurations the valve 212 can be situated mainly within the frame 202. In this case, the spring 216 could be attached to the frame and the seal 214 can be positioned on the same side of the valve seat 218 as illustrated, or on the opposing side of the valve seat 218.

In some embodiments, the cell culture container 200 can include another port 222 that is situated on a different adjacent side of the cell culture container 200 as a port 208. In some cases, the port 222 can include a bore 224 directed through the frame 202, and a check valve 226 that includes a valve seat that can be coupled to or integrally formed with the membrane 204 (e.g., in a similar manner as the valve seat 218). The check valve 226 is configured to only allow fluid to pass in a direction that extends through the bore 224, through the check valve 226, and into the interior volume 206 of the cell culture container 200. As such, fluid is blocked from flowing through the check valve 226 in a direction from the interior volume 206 of the cell culture container 200 and to the bore 224.

FIG. 5 shows a cross-sectional view of the cell culture container 200 engaged with a flow coupler 230 and with the valve 212 in an actuated position. The flow coupler 230 is a specific configuration of the flow coupler 110. The flow coupler 230 can include a reciprocating member 232 with a bore 234 directed therethrough, an engagement feature 236, and a head 238 that is positioned at an end of the reciprocating member 232. The engagement feature 236 of the flow coupler 230 is coupled to the reciprocating member 232 and can engage with the extension 220 and the frame 202 to generate a seal between the frame 202. In some cases, a sealing layer (e.g., a gasket) can be positioned at the end surface of the engagement feature 236 to generate a seal between the frame 202 and the end surface of the engagement feature 236. The engagement feature is illustrated as being an extension off the reciprocating member 232 that encapsulates the extension 220 and the bore 210. Additionally, the engagement feature 236 has a void that receives the extension 220.

As the reciprocating member 232 is advanced (e.g., by an electric motor, pneumatically, spring operation, etc.) towards the cell culture container 200, the reciprocating member 232 travels through the bore 210, contacts the seal 214 of the valve 212 until the seal 214 moves away from the valve seat 218 and the valve 212 opens. Once the valve 212 is opened, fluid within the interior volume 206 can travel through the head 238, into and up through the bore 234 to the receptacle 104 and/or to cell processing modules 114,116 container, or to the fluid handling device 105. Because the interface between the bore 210 and the reciprocating member 232 is relatively tight, and a seal is provided between the engagement feature 236 and the frame 202, the fluid within the interior volume 206 is protected from the ambient environment.

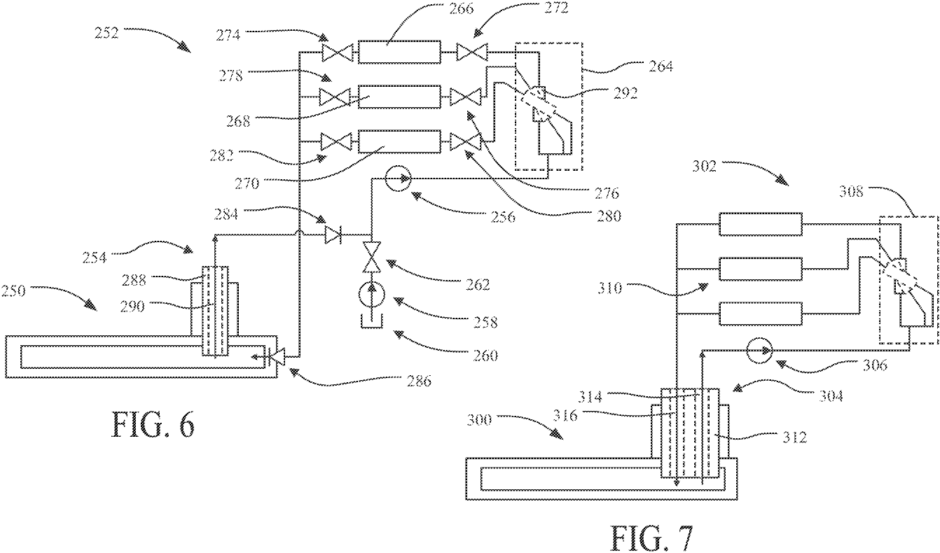

FIG. 6 shows a schematic illustration of one embodiment of a cell culture container 250, engaged with a receptacle 252. Both the cell culture container 250 and the receptacle 252 are specific configurations of the cell culture container 102 and the receptacle 104, respectively. The cell culture container 250 may include components and features as the other previously described cell culture containers, and thus the components and features of the other previously described cell culture containers may pertain to the cell culture container 250. As shown, the receptacle 252 includes a flow coupler 254, pumps 256, 258, a reservoir 260, an adjustable valve 262 downstream of pump 258, an adjustable valve 264 downstream of pump 256, cell processing modules 266, 268, 270, and valves 272, 274, 276, 278, 280, 282 positioned upstream and/or downstream of cell processing modules, 266, 268, and 270.

The flow coupler 254 is a specific configuration of the flow coupler 110, and can include an internal reciprocating member 288 and a bore 290 directed therethrough. The internal reciprocating member 288 of the flow coupler 110 may engage the interior volume of the cell culture container 250 to establish flow from the cell culture container 250 through the bore 290 of the flow coupler and/or to establish flow to the cell culture container 250 through the bore 290 of the flow coupler. The bore 290 of the flow coupler 254 may be in fluid communication with the pump 256, which may be a syringe pump, that draws fluid out from the interior volume of the cell culture container 250 and/or that introduces fluid into the interior volume of the cell culture container 250. The adjustable valve 264 has a single flow path 292 that may be moved to selectively place either of the cell processing modules 266, 268, 270 into fluid communication with the bore 286 of the flow coupler 254 (and thus the interior volume of the cell culture container 250). As illustrated, the flow path 292 of the adjustable valve 264 is in fluid communication with the cell processing module 266 and is not in fluid communication with the cell processing modules 268, 270 for this valve position. However, different valve positions of the adjustable valve 264 can adjust which cell processing module is selected for use. For example, by rotating the flow path 292 in a counterclockwise direction (e.g., relative to the view in FIG. 6), the flow path 292 is removed from alignment with the flow path of the cell processing module 266 to be in alignment with the flow path of the cell processing module 268. In this case, the flow path 292 is in fluid communication with the cell processing module 268 and is not in fluid communication with the cell processing modules 266, 270. In this embodiment, the selectable configuration ensures that only one cell processing module is used at a time.

Although the adjustable valve 264 is illustrated as a rotary valve, where the rotational position of the single flow path 292 may be adjusted to selectively which cell processing module 266, 268, 270 is in fluid communication with the bore 286 of the flow coupler 254 (and the pump 256), in other configurations the adjustable valve 264 may be configured to move and be adjusted in a manner other than rotationally in order to select which cell processing module 266, 268, 270 is in fluid communication with the bore 286 of the flow coupler 254 (and the pump 256). Additionally, although the adjustable valve 264 is illustrated as having a single movable flow path 292, in other configurations, the adjustable valve 264 may have a plurality of flow paths where each of the plurality of flow paths is dedicated to a single cell processing module 266, 268, 270. In this embodiment, different rotational positions of the valve would align one flow path with the corresponding cell processing module to place the cell processing module in fluid communication with the bore 286 of the flow coupler 254 (and the pump 256), while the other remaining flow paths would not be in fluid communication with the bore 286 of the flow coupler 254 (and the pump 256). In this way, only one of the cell processing modules 266, 268, 270 will be in fluid communication with the bore 290 of the reciprocating member 288 at a time.

In some configurations, the system 100 may include a reservoir 260. The reservoir optionally may be contained in the receptacle 252 or in the fluid handling device 105. The system further may include a pump 258 and one or more valves 262, 284, which optionally may be a solenoid valve, or a pinch valve. In some cases, to replenish fluid within the fluid circuit, the valve 262 can be selectively opened (e.g., by a computing device), and the pump 258 can be activated (e.g., by a computing device) to draw fluid from the reservoir 260 and into the flow path. In some cases, the valve 284 may be configured as a check valve preventing back flow of fluid into the bore 286 of the flow coupler 254 (and into the interior volume of the cell culture container 250). The reservoir may contain one or more of reagents, and/or cells for culturing in the cell culture container 250. Non-limiting examples of reagents include media, cell differentiation factors, immune cell activation factors, viruses for viral transduction, RNA, DNA, beads, polypeptides, small molecules, chemical reagents such as glucose etc. In some embodiments, the reservoir 260 includes fresh media which is utilized to replace spent media in the cell culture container 250. In this case, the spent media and cells may be removed from the cell culture container 250 via actuating the reciprocating member 288 of the flow coupler 254 and passing the spent media and cells through the bore 286 of the flow coupler 254 in a flow path to a cell processing module 266, 268, 270 in the receptacle 252. The cell processing module 266, 268, 270 may separate the cells from the spent media and pass the spent media through a flow path to a vessel where the spent media may be contained and optionally disposed. Fresh media then may be transferred from the reservoir 260 to the cell processing module 266, 268, 270, where the fresh media is utilized to suspend and transfer the cells from the cell processing module 266, 268, 270 back to the cell culture container 250, which optionally may have been replaced with a fresh cell culture container. In other embodiments, the cells may be transferred from the cell processing module 266, 268, 270 to a container other than the cell culture container 250 (e.g., a storage container for freezing the cells).

In some cases, each of the flow paths of the cell processing modules 266, 268, 270 can have valves positioned on opposing ends, or in other words, one valve positioned upstream of the inlet and/or one valve positioned downstream of the outlet. For example, the cell processing module 266 can have the valve 272 positioned upstream of the inlet of the cell processing module 266 and the valve 274 positioned downstream of the outlet of the cell processing module 266. Similarly, the cell processioning module 268 can have the valve 276 positioned upstream of the inlet of the cell processing module 268 and the valve 278 positioned downstream of the outlet of the cell processing module 268, and the cell processing module 270 can have the valve 280 positioned upstream of the inlet of the cell processing module 270 and the valve 282 positioned downstream of the outlet of the cell processing module 270. Each of the valves 272, 274, 276, 278, 280, 282 can be adjustable opened and closed (e.g., by a computing device), where fluid is allowed to flow through the valve when open, and fluid is prevented from flowing through the valve when closed. Thus, in some cases, the valves 272, 274, 276, 278, 280, 282 can be implemented as solenoid valves or pinch valves.

The valves 272, 274, 276, 278, 280, 282 may ensure that fluid does not pass through a respective cell processing module 266, 268, 270 while the respective cell processing module 266, 268, 270 has not been selected for use, or while the respective cell processing module 266, 268, 270 has been selected for use. For example, in the illustrated embodiment, all the valves 274, 276, 278, 280, 282 may be closed and the valve 272 may be opened. This ensures that fluid (which optionally includes cells) flows into cell processing module 266 and that fluid (which optionally includes cells) does not flow into and through the cell processing modules 268, 270. After fluid is received in the cell processing module 266, the upstream valve 272 optionally may be closed to isolate the fluid to only the cell processing module 266. Then, the process defined by the cell processing module 266 may be performed. After the process performed by the cell processing module 266 is completed, the downstream valve 274 may be opened (and if the upstream valve 272 was closed, the upstream valve 272 may be opened) and the fluid (which optionally includes cells) may be transferred from the cell processing module 266 back into the interior volume of the cell culture container 250, into a different container (e.g., a storage container), or into another cell processing module 268, 270 for further processing.

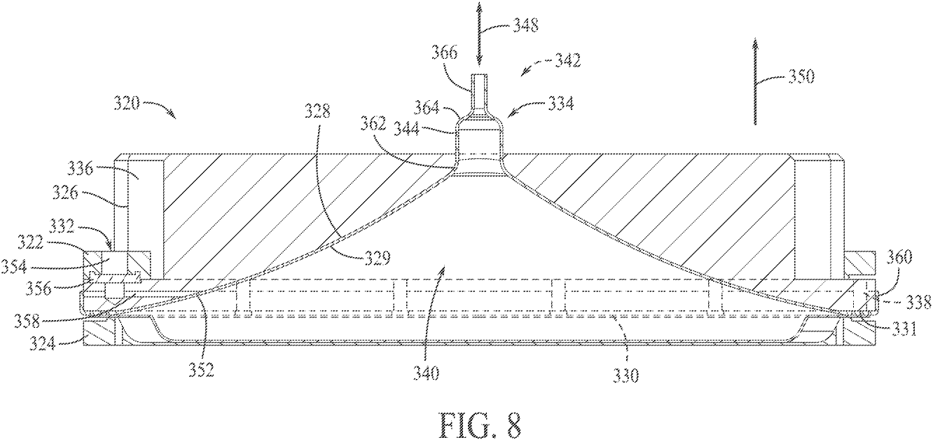

FIG. 7 shows a schematic illustration of an example of a cell culture container 300 engaged with a simplified receptacle 302, both of which are specific examples of the cell culture container 250 and the receptacle 104, respectively. The cell culture container 300 may include similar components and features as the other previously described cell culture containers, and thus the components and features of the other previously described cell culture containers may also pertain to the cell culture container 300. As shown, the receptacle 302 can include a flow coupler 304, a pump 306, an adjustable valve 308, and a plurality of cell processing modules 310. The flow coupler 304 is a specific configuration of the flow coupler 110 and can include a reciprocating member 312 and dual bores 314, 316 directed therethrough. In some configurations, this dual bore configuration of the flow coupler 304 allows one of the bores 314, 316 to define an inlet, and the other of the bores 314, 316 to define an outlet. Fluid including cells from the interior volume of the cell culture container 300 is drawn, by the pump 306, up through bore 314 through the adjustable valve 308, and through one of the cell processing modules 310. Then, after all the cell processing has been completed, the fluid including the processed cells can be pumped through the bore 316 and back into the interior volume of the cell culture container 300. This dual bore configuration of the flow coupler 304 can be advantageous in that the cell culture container 300 only needs a single port, rather than having multiple ports.

In some configurations, the pump 306 can be a reversible pump. In this case, the flow coupler 304 can have a single bore (e.g., one of the bores 314, 316). Then, when the flow coupler 304 comes into fluid communication with the interior volume of the cell culture container 300 fluid is drawn by the fluid into one of the cell processing modules 310. Then, when cell processing is completed, the cells can be pumped in the opposing direction back through the single bore of the flow coupler 304 and back into the interior volume of the cell culture container 300.

FIG. 8 shows a front cross-sectional view of a centrifuge container 320, which in some cases, can be a specific implementation of the cell culture container (e.g., the cell culture container 130). For example, along with centrifuging cells within the centrifuge container 320, the centrifuge container 320 can be configured to facilitate growing (and multiplying) of cells within the centrifuge container 320. As shown in FIG. 8, the centrifuge container 320 can include frames 322, 324, a tub 326, a centrifuge structure 328 (e.g., a centrifugation slope) having peripheral surfaces 329, a plate 331 (or optionally a membrane 330), and ports 332, 334. The frames 322, 324 can be structured in a similar manner as the pieces 134, 138 of the cell culture container 130. For example, the frame 322 can be structured similar to the piece 134, while the frame 324 can be structured similar to the piece 138. In some cases, each frame 322, 324 can include a first plurality of holes (e.g., some or all of which can be threaded) to receive one or more fasteners (e.g., threaded fasteners) to couple the frames 322, 324 together, and a second plurality of holes to receive one or more pins to secure the centrifuge container 320 to, for example, a fluid handling device, etc., as described below. In addition, each frame 322, 324 can include a hole directed therethrough to receive a component of the centrifuge container 320. For example, a portion of the tub 326 can be received through the hole of the frame 322.

The tub 326 can define a bowl 336, and a peripheral extension 338 emanating from the bowl 336. As described in more detail below, the interior of the bowl 336, can at least partially define an interior volume of the centrifuge container 320. In some cases, the height of the bowl 336 can be larger than the thickness of either or both of the frames 322, 324. In this way, with the tub 326, the centrifuge container 320 can provide a considerable increase in the interior volume (as compared to the cell culture container 130), which can provide a larger space for growing cells, and allow for different shapes for centrifuge structure 328 (e.g., to facilitate the centrifuge process), etc. While the bowl 336 of the tub 326 is illustrated as having a rectangular shape, in other configurations, the bowl 336 can have other shapes. Correspondingly, for example, the peripheral extension 338 can have a shape that corresponds to the shape of the bowl 336 (and the shape of the frames 322, 324), which in the illustrated embodiment is a rectangle, however, alternative shapes are contemplated.

In some embodiments, similarly to the frames 322, 324, the peripheral extension 338 of the tub 326 can include a plurality of holes each of which can align with a hole of each of the frames 322, 324 to facilitate coupling the components together (e.g., to receive a threaded fastener). Thus, in some cases, the plurality of holes of the peripheral extension 338 can be threaded. As shown in FIG. 8, at least a portion of the tub 326 can be sandwiched between the frames 322, 324. For example, the peripheral extension 338 can be positioned between frames 322, 324, with the bowl 336 of the tub 326 being received through the hole of the frame 322 (e.g., which can correspond to the peripheral shape of the bowl 336). In some cases, the tub 326 can include a hole 342 for receiving at least a portion of the port 334.

The membrane 330 can be implemented in a similar manner as the previously described membranes (e.g., the membranes 140, 168). For example, the membrane 330 can be a gas permeable membrane, which can facilitate movement of gas through the membrane 330, but which blocks movement of liquids through the membrane 330. As a more specific example, the membrane 330 can be a formed out of silicone. In other cases, the membrane 330 can be formed out of a polymer (e.g., a plastic, including polypropylene, polycarbonate, etc.). Regardless of the configuration, membrane 330 can be configured to minimally bind cells to the surface of the membrane 330 (e.g., ideally binding no cells at all to the surface of the membrane 330). As shown in FIG. 8, the membrane 330 can be positioned within the interior volume of the bowl 336, and can partially define the interior volume 340 of the centrifuge container 320 in which cells (and cell media) are contained.

In some embodiments, the membrane 330 can be sandwiched between the frames 322, 324, and in particular can be sandwiched between the frame 322 and the centrifuge structure 328. Similarly to the frames 322, 324, the membrane 330 (e.g., a peripheral flange of the membrane 330) can include a plurality of holes, which can align with other holes of the frames, 322, 324, the tub 326, and the centrifuge structure 328 to ensure that the membrane 330 is properly clamped and secured during the centrifuge process (or during growing cells in the centrifuge container 320). For example, each of these holes of the membrane 330 can receive a respective threaded fastener (e.g. to be received in secured to the frame 324).

In some embodiments, the centrifuge structure 328 can be coupled to (or integrally formed with) a body 344 of the port 334. For example, the centrifuge structure 328 can include a first hole positioned near a central region of the centrifuge structure 328, which can receive the body 344 of the port 334 to couple the body 344 to the centrifuge structure 328 at the first hole (e.g., using an adhesive). In some cases, the centrifuge structure 328 can include a plurality of other holes surrounding the first hole of the centrifuge structure 328 which can align with the holes of the other components (e.g., the frames 322, 324, the tub 326, the membrane 330, etc.).

In some embodiments, the centrifuge structure 328 can have a peripheral surface 329, which can be defined between the first hole of the centrifuge structure 328 and a peripheral edge of the centrifuge structure 328 and can have a non-planar shape. For example, the peripheral surface 329 can be curved, angled, etc., partially (or entirely) around an axis 348 that extends through the port 334 and through the frames 322, 324. In addition, the peripheral surface 329 of the membrane 330 can be angled, curved, etc., towards the first hole of the centrifuge structure 328 and relative to the axis 348. In some configurations, the axis 348 can be perpendicular to a horizontal surface of each of the frames 322, 324. In some embodiments, and as illustrated in FIG. 8, the interior volume 340 of the centrifuge container 320 can be defined between the peripheral surface 329 of the centrifuge structure 328 and the plate 331 (or the membrane 330, for example, if the centrifuge container 320 includes the membrane 330 rather than the plate 331). In some embodiments, the centrifuge structure 328 can be inserted into the interior volume of the tub 326, and can be clamped between the frames 322, 324. For example, a peripheral end of the centrifuge structure 328 that can include a plurality of holes can be positioned between the frames 322, 324, positioned under the peripheral end 338 of the tub 326, and can be above the plate 331 (or the membrane 330). In this way, each hole of the plurality of holes of the centrifuge structure 328 can align with a respective hole of the frame 322, the frame 324, the peripheral extension 338, and the plate 331 (or membrane 330) and receive a fastener (e.g., a threaded fastener) to couple the centrifuge structure 328 to the tub 326, the frames, 322, 324, and the plate 331 (or the membrane 330).

Regardless of the configuration of the peripheral surface 329 of the centrifuge structure 328, the cross-sectional area defined by the peripheral surface 329 of the centrifuge structure 328 can decrease in a direction from the frame 324 and towards the port 334 along the axis 348. In this way, when a centrifugal force 350 is applied to the centrifuge container 320, which can extend in a direction along the axis 348 (or an axis parallel to the axis 348) upwardly, cells are forced into and form a pellet within the body 344 of the port 334 (e.g., as the cells travel along the peripheral surface 329). In some embodiments, the interior volume 340 of the centrifuge container 320 can be in fluid communication with the port 332 so that cells, media, etc., can be introduced though the port 332, through the centrifuge structure 328, and into the interior volume 340. Correspondingly, cells, media, etc., can be forced to flow along a flow path from the interior volume 340, through the centrifuge structure 328, and through the port 332.

In some embodiments, the centrifuge structure 328 can include a port 352 that can align with a portion of the port 332 to allow fluid communication between the interior volume 340 and the port 332. For example, the port 332, which can be structured in a similar manner as the port of other cell culture containers described herein, can include a hole 354, a septum 356, and a conduit 358. The hole 354 can be directed through the frame 322, while the conduit 358 can be directed through the tub 326. The hole 354 can be aligned with the conduit 358 to fluidly connect the components. However, the septum 356 can span across a portion of the hole 354 to fluidly isolate the hole 354 from the conduit 358. In some cases, when the septum 356 is pierced (e.g., by a needle), the needle (and components upstream of the needle) are brought into fluid communication with the conduit 358, and thus the interior volume 340 (e.g., through the port 352).

As shown in FIG. 8, the centrifuge container 320 includes both the membrane 330 and the plate 331. However, it should be appreciated that, the centrifuge container 320 can include either the membrane 330 or the plate 331. For example, in one case, the membrane 330 can provide the lower boundary for the interior volume 340, while in a second case, the plate 331 can provide a lower boundary for the interior volume 340.

In some embodiments, the plate 331 can be planar with an upwardly extending peripheral flange 360. However, in other configurations, the plate 330 can have other three-dimensional shapes (e.g., the plate 330 being curved). Similarly to the frames 322, 324, the tub 326, etc., a peripheral end of the plate 331 can include a plurality of holes each of which can align with a respective hole of the frames 322, 324, and the tub 326, and can subsequently receive a fastener (e.g., a threaded fastener) to couple the components together. As shown in FIG. 8, the plate 331 can be positioned between the frames 322, 324, and can be situated underneath the tub 326. In particular, the peripheral flange 360 of the plate 331 can extend upwardly along the peripheral extension 338 of the tub 326 so that the tub 326 is restricted from moving relative to the plate 331 (e.g., by contacting the peripheral flange 360).

In some embodiments, the body 344 of the port 334 can include sections 362, 364, 366. The section 362 is situated below the sections 364, 366 and can include a local minima in cross-section and a flared end that increases in cross section away from the port 334 and towards the frame 322. Thus, the flared end of the section 362 has a larger cross-section than the cross-section of the local minima of the section 362. In some cases, the first hole of the centrifuge structure 328 can be positioned (and coupled) at the local minima of the section 362, which can prevent the centrifuge structure 328 from sliding off the body 344 (e.g., due to the flared end of the section 362). The section 364 can be positioned between the sections 362, 364, and can have a larger cross-section than the section 366. In some cases, the body 344 of the port 334 can be coupled to the bowl 336 of the tub 326 at the hole 342, and a portion of the body 344 can be positioned on an exterior side of the tub 326. The section 366 can be positioned above the sections 362, 364, and the entire section 366 can be positioned on an exterior side of the tub 326. The cross-section of the section 366 can be smaller than the cross-section of the sections 364, 366, which can facilitate receiving and compacting a cell pellet during the centrifuging process. For example, when the centrifugal force 350 is applied to the centrifuge container 320, the cells traverse the sections 362, 364, until being forced into the section 366 to form a cell pellet. In some cases, and as described below, after the cell pellet is formed in the section 366 of the body 344, the pellet can be extracted through the port 334, the cell pellet can be resuspended (e.g., in other cell media) after, for example, the cell media has been exchanged, etc. In some cases, after the cell pellet is formed, the port 332 can be used to extract the (spent) cell culture media from the interior volume 340, dispense new cell culture media into the interior volume 340 (e.g., via the port 332), and resuspend the cells from the cell pellet (e.g., by introducing cell culture media into the port 334).

In some embodiments, including when the membrane 330 defines the lower boundary of the interior volume 340 and if the membrane 330 is gas permeable, then the centrifuge container 320 can be placed in a standard incubator (e.g., the centrifuge container 320 being a closed system cell culture vessel). Alternatively, if the membrane 330 of the centrifuge container 320 is not gas permeable (or the plate 331 is utilized as the lower boundary of the interior volume 340), then the centrifuge container 320 may not be a closed system cell culture container. In this case, cell media may need to be exchanged more frequently. In some embodiments, the interior volume 340 of the centrifuge container 320 (or other cell culture containers) can be in a range of substantially 15 mL to substantially 750 mL, in a range of substantially 50 mL to substantially 700 mL, in a range of substantially 100 mL to substantially 600 mL, in a range of substantially 200 mL to substantially 500 mL, etc. In some cases, the interior volume 340 of the centrifuge container 320 can be substantially 50 mL, substantially 200 mL, or substantially 500 mL.

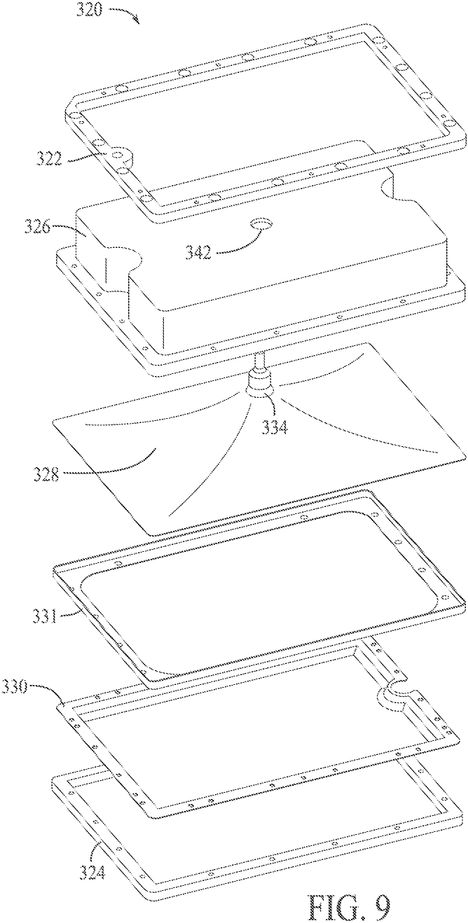

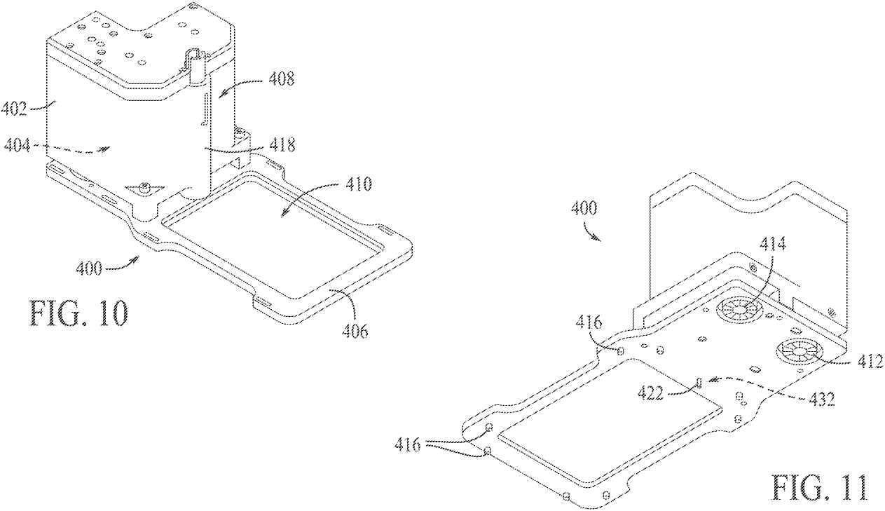

FIG. 9 shows an exploded view of the centrifuge container 320. In some cases, to assemble the centrifuge container 320 as illustrated in FIG. 8, the tub 326 is positioned so that the bowl 336 faces upwards, and the peripheral extension 338 faces downwards. In other words, the tub 326 is positioned so that the bowl 336 is positioned above the peripheral extension 338. Then, the frame 322 can be placed around the bowl 336 of the tub 326, and the plate 331 (or the membrane 330) can be positioned under the tub 326 with the peripheral surface 329 positioned between the plate 331 (or the membrane 330) and the tub 326. After, the frame 324 can be positioned underneath the plate 331 (or the membrane 330), and each hole (e.g., fastening hole) of each of the frame 322, the tub 326, the plate 331 (or the membrane 330), and the frame 324 can be aligned can receive a fastener to couple these components together.

FIG. 10 shows a top isometric view of a fluid handling device 400 comprising a receptacle for receiving a cell culture container (e.g., the cell culture container 130, or the cell culture container 160), and FIG. 11 shows a bottom isometric view of the fluid handling device 400. The fluid handling device 400 is a specific configuration of the fluid handling device 105 and can include a housing 402 having an interior volume 404 therein, an extension 406 extending from the housing 402, and a flow coupler 408. The interior volume 404 of the housing 402 can secure and enclose one or more cell processing modules, as described below. The extension 406 includes a centrally located aperture 410, that when engaged with a cell culture container, allows the membrane of the cell culture container to extend through the aperture 410. The flow coupler 408 is a specific configuration of the flow coupler 110 and is described in more detail below.

As shown in FIG. 11, the fluid handling device 400 also includes multi-position adjustable valves 412, 414 that can each be used to adjust the flow paths of fluid within the fluid handling device 400. The adjustable valves 412, 414 may be manually adjustable and/or may be adjusted via mechanical/electronic components. The adjustable valves 412, 414 may be adjusted to select which cell processing module is to be used (and which are not to be used) in a similar manner as the adjustable valve 264 of FIG. 6. In certain embodiments, the adjustable valves may be used to create a configurable fluidic path for routing cells and reagents through the cell culture container 130, 160 and the cell processing module 114, 116 to perform a cell process. In some embodiments, the adjustable valves on the cell processing module 114, 116 connect to the fluid handling device 105 through a matching actuator valve located on the fluid handling device 105. In some embodiments, the position of each of the adjustable valves 412, 414 can interface with and can be adjusted by a computing device which optionally is present in the fluid handling device 105.

In some embodiments, the fluid handling device 400 includes alignment features that engage with corresponding alignment features of the cell culture container to align a port of the cell culture container with a bore of the flow coupler 408. In particular, the fluid handling device 400 may include downwardly extending pins 416. Each pin 416 may engage with a corresponding channel of the cell culture container (e.g., the bores 154 of the cell culture container 130). As shown, the pins 416 are situated on opposing ends of the aperture 410 of the extension 406 so that when a cell culture container is interfaced with the fluid handling device 400 some pins 416 engage with some channels on one side of the cell culture container, and other pins 416 engage with other channels an opposing side of the cell culture container, which can provide a stable interface between the fluid handling device 400 and the cell culture container.

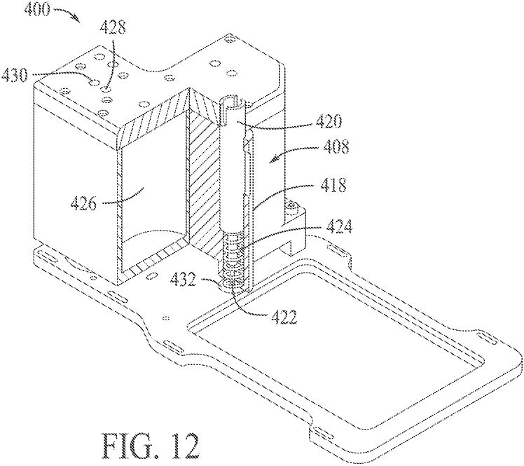

FIG. 12 shows another perspective view of the fluid handling device 400 comprising the receptacle, with portions of the fluid handling device 400 opened for visual clarity. As shown, the flow coupler 408 includes a flow coupler housing 418, a reciprocating member 420, a needle 422 attached to the reciprocating member 420 at an end thereof, a spring 424, a reagent reservoir 426, a inlets/outlets 428, 430, a barrier 432, and an actuatable stop (not shown). The barrier 432 is coupled to (or integrated within) the end of the flow coupler housing 418 so that the barrier 432 extends entirely across a bore that extends through the flow coupler housing 418. The barrier 432 ensures that the needle 422 is sterile prior to usage of the needle 422, and thus the barrier 432 provides a sterilized barrier for the needle 422. In some cases, the barrier 432 can be a septum or a removable seal (e.g., an adhesive backed foil or a polymeric material seal such as a rubber seal). In some cases, after the barrier 432 has been perforated during usage of the needle 422, the needle 422 may be retracted.

In some embodiments, the fluid handling device 400 includes one or more inlets/outlets 428/430 which may be utilized to couple the fluid handling device 400 to a fluid handling device 105. The inlets/outlets 428,430 may comprise barriers to prevent exposing the closed cell processing system to ambient conditions. For example, suitable barriers may include filters having a pore size of less than about 0.22 microns (e.g., PTFE filter membranes) which may allow gas equilibration during reagent loading and/or liquid motion during a unit operation, while ensuring that the cell processing system remains closed to microbial contaminants.

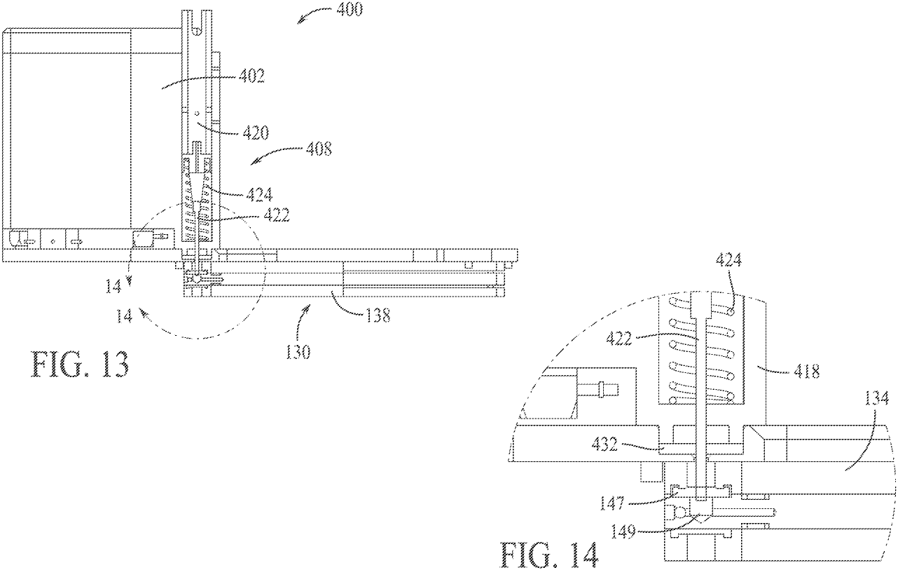

FIG. 13 shows a cross-sectional view of the fluid handling device 400 engaged with the cell culture container 130 and with the flow coupler 408 (e.g., a self-sterilizing connection) actuated. FIG. 14 shows an enlarged cross-sectional view of FIG. 13 that details the engagement between the flow coupler 408 and the cell culture container 130. During storage, the reciprocating member 420 and the needle 422 are raised and biased with the spring 424 so that the needle 422 is above the barrier 432. In this state, an actuatable stop (not shown) can be advanced (e.g., by the computing device) to be positioned under a portion of the reciprocating member 420. In this way, the actuatable stop can maintain the biased position of the flow coupler 408. In some embodiments, prior to loading the needle 422 (and sealing with the barrier 432) such as between uses, the needle 422 can be sterilized (e.g., by autoclaving, gamma radiation, ethylene oxide, an alcohol or peroxide solution, such as 70% isopropyl alcohol or 70% hydrogen peroxide, etc.). In some embodiments, prior to actuating the flow coupler 408, surfaces that are to come in contact with each other after actuation of the flow coupler 408 can be sterilized. For example, a lower surface of the barrier 432 and an upper surface of a septum 147 of the port 146 of the cell culture container 130 can be sterilized (e.g., with isopropyl alcohol).

Once appropriately sterilized, the reciprocating member 420 including the needle 422 can be advanced until the needle 422 punctures and extends through both the barrier 432 and the septum 147 and enters into a conduit 149 of the cell culture container 130 that is in fluid communication with the interior volume of the cell culture container 130. In some embodiments, such as when the flow coupler 408 includes the actuatable stop, the actuatable stop can be retracted (e.g., by the computing device) until the actuatable stop is removed from contact with the reciprocating member 420. At this point, because the reciprocating member 420 is spring-loaded, the needle 422, driven by the spring force, advances and punctures the barrier 432 and the septum 147. In some cases, this spring biased actuation of the needle 422 allows for a more quick and forceful puncturing of the barrier 432 and the septum 147, which can provide a better seal between the needle 422 and the barrier 432 or the septum 147. In other configurations, however, the needle 422 can be electrically or pneumatically advanced to puncture the barrier 432 and the septum 147. Once the needle 422 is inserted and in fluid communication with the interior volume of the cell culture container 130, fluid can be pumped from the interior volume and upwardly through a flow path that is defined by the needle 422 and the reciprocating member 420 to a different flow path of the fluid handling device 400 (e.g., the different flow path being in fluid communication with a cell processing module) or directly to a different flow path of the cell processing module.

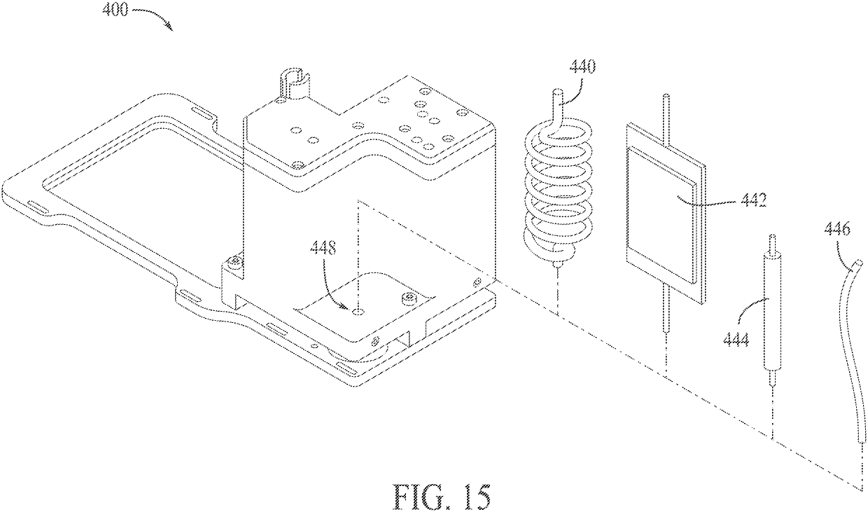

FIG. 15 shows a rear perspective view of the fluid handling device 400 with different cell processing modules 440, 442, 444, 446, each of which may be inserted in the fluid handling device 400. Each of the cell processing modules 440, 442, 444, 446 may define a cell process and/or a combination of the cell processing modules together may define a cell process. Each of the cell processing modules 440, 442, 444, 446 has a flow path, in which one end of the flow path connects to a corresponding port 448 of the fluid handling device 400 and an opposing end of the flow path connects to a different port of the fluid conduit and/or to a port of the fluid handling device 105 to establish a fluid circuit within the cell processing system 100. The port 448 of the fluid handling device 400 may be in fluid communication (or selective fluid communication) with a port of the cell culture container (e.g., the port 148 of the cell culture container 130).

The cell processing modules 440, 442, 444, 446 each can provide a unique function for cells as they flow along the flow path of a cell processing module 440, 442, 444, 446. For example, the cell processing module 440 is a spiral attachment with a spirally wound flow path that can detach and capture magnetic beads from the cell culture media (e.g. magnetic beads that are bound to components of the cell culture media which may include cells). As another example, the cell processing module 442 can include electrodes that can be positioned on opposing sides of the flow path of the cell processing module 442, and that can be energized to apply an electric field that is substantially (e.g., deviating by less than 20%) perpendicular to the flow path of the cell processing module 442 to provide electroporation to cells in the flow path. As yet another example, the cell processing module 444 can include a conduit with a larger surface area and volume, which can be used for magnetic cell isolation/enrichment as the cells pass through the conduit. As still yet another example, the cell processing module 446 can be a conduit that provide a simple flow through connection, which can be used for cell transfer and/or cell media exchange.

In some embodiments, the multiple cell processing modules 440, 442, 444, 446 may be connected in series within a flow circuit of the cell processing system 100 (e.g., wherein fluid flow passes from one cell processing module to another cell processing module). In other embodiments, the multiple cell processing modules 440, 442, 444, 446 may be connected in parallel within a flow circuit of the cell processing system 100. In this case, one end of each flow path of each cell processing module 440, 442, 444, 446 may interface with an adjustable valve 412, and the adjustable valve 412 may be adjusted to establish fluid flow through a selected cell processing module 440, 442, 444, 446 and close fluid flow through the non-selected cell processing modules 440, 442, 444, 446. The adjustable valve 412 may be utilized to establish a flow circuit between the cell culture container 130, the fluid handling device 400, the one or more cell processing modules 440, 442, 444, 446, and the fluid handling device 105. In certain embodiments, only one of the multiple cell processing modules 440, 442, 444, 446 may be connected in within a flow circuit of the cell processing system 100 at a time.

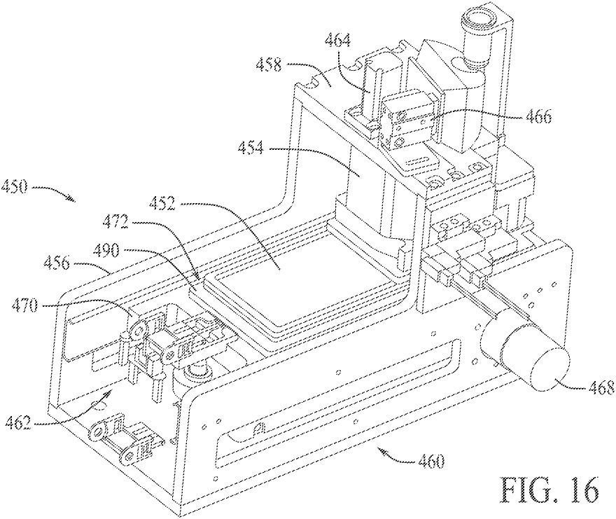

FIG. 16 shows a front isometric view of a fluid handling device 450 engaged with a cell culture container 452, and a cell processing module 454. In some embodiments, the cell culture container 452 can be a specific implementation of any of the previously described cell culture containers. In addition, the cell culture container 452 can be replaced with a centrifuge container (e.g., the centrifuge container 320). In some embodiments, the cell processing module 454 can also be a specific implementation of the previously described cell processing modules.

In some embodiments, the fluid handling device 450 can include a housing 456 including a top plate 458, a shuttle assembly 460, a clamping assembly 462, an actuation assembly 464 for piercing a septum (e.g., of the cell culture container 452), and a magnet assembly 466. The shuttle assembly 460 can include an actuator 468, and a moveable rack 470 in engagement with the actuator 468. The actuator 468 can be configured to extend the moveable rack 470 along a first direction, and can extend the moveable rack 470 along a second direction opposite the first direction. As shown in FIG. 16, the moveable rack 470 can support the cell culture container 452, and the cell processing module 454. Thus, movement of the moveable rack 470 can also move the cell culture container 452, and the cell processing module 454. In some cases, the moveable rack 470 can include engagement features 472, 474. The engagement feature 472 can contact (and engage) the cell culture continuer 452 to ensure that the cell culture container 452 is positioned at a repeatable location on the moveable rack 470. In some cases, the engagement feature 472 can include a tray with a recess that is coupled to the moveable rack 470 and that the recess of the tray receives and retains the cell culture container 452. Similarly, the engagement feature 474 can contact a housing 455 of the cell processing module 454 to ensure that the cell processing module 454 is positioned at a repeatable location on the moveable rack 470. In particular, the engagement feature 474 can ensure that the cell processing module 454 is aligned properly with the cell culture container 452, and is aligned properly with a flow path of the fluid handling device 450.

In some embodiments, the actuator 468 can extend (and retract) thereby extending (and retracting) the moveable rack 470. In some configurations, the actuator 468 can be a linear actuator, while in other configurations, the actuator 468 can be a pneumatic actuator. For example, in the illustrated embodiment, the actuator 468 can be a linear actuator that includes a motor (e.g., an electric motor) with a rotatable shaft coupled to a pinion gear. Correspondingly, the moveable rack 470 can also define a portion of the actuator 468, with the moveable rack 470 including a plurality of teeth along a longitudinal dimension of the moveable rack 470. As the pinion gear of the actuator 468 rotates in a first rotatable direction, the moveable rack 470 moves in the first direction, while as the pinon gear of the actuator 468 rotates in a second rotatable direction, the moveable rack 470 moves in the second direction.

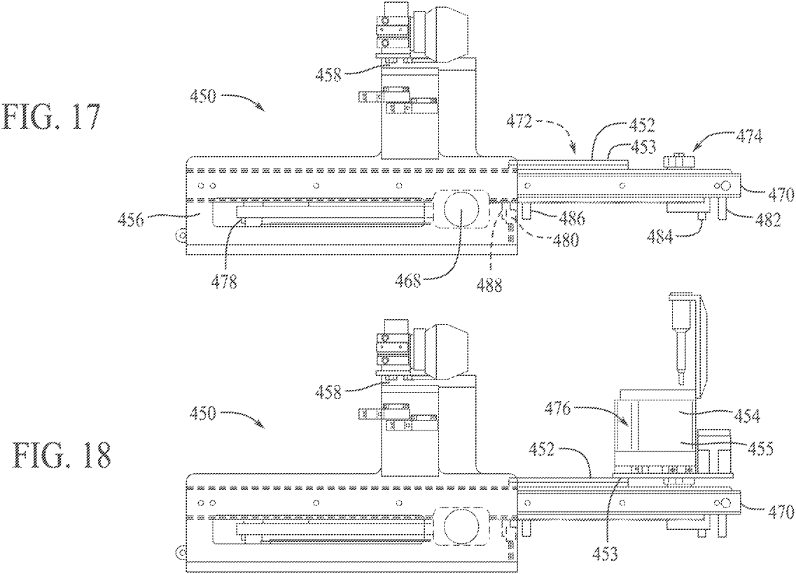

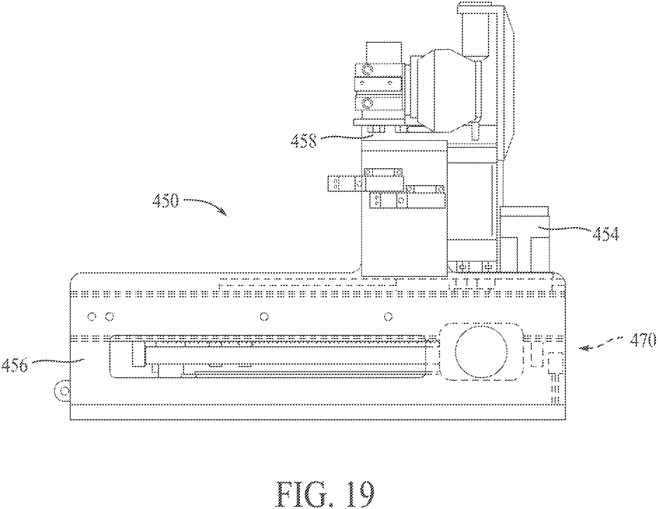

FIG. 17 shows a partial side view of the fluid handling device 450 with the moveable rack 470 positioned in an open configuration with the cell culture container 452 in contact with the engagement feature 472, and with the cell processing module 454 removed from the fluid handling device 450. FIG. 18 also shows a partial side view of the fluid handling device 450 with the moveable rack 470 in an open configuration, but with the cell processing module 454 and the cell culture container 452 supported by the moveable rack 470. For example, the housing 455 of the cell processing module 454 is in contact with the engagement feature 474 to constrain the movement between the cell processing module 454 and the moveable rack 470, and to ensure that the cell processing module 454 is aligned with the port 453 of the cell culture container 452. For example, when a portion of the housing 455 contacts the engagement feature 474, a flow coupler 476 of the cell processing module 454 (which can be similar to the flow coupler 408) aligns with the port 453 of the cell culture container 452. In particular, the flow path defined by a needle of the flow coupler 476 aligns with the port 453 of the cell processing culture container 452 when the housing 455 of the cell processing module 454 contacts the engagement feature 474. In this way, with the cell culture container 452 in contact with (and secured to) the engagement feature 472 and with the housing 455 of the cell processing module 454 in contact with (and secured to) the engagement feature 472, even if the moveable rack 470 moves, the flow coupler 476 still is in alignment with the port 453. In some embodiments, the engagement feature 474 can be implemented in different ways. For example, the engagement feature 474 can include a post that is inserted into a recess of the housing 455 of the cell processing module 454.

FIG. 19 show a partial side view of the fluid handling device 450 with the moveable rack 470 in a closed configuration, in which the moveable rack 470 is supporting the cell culture container 452 and the cell processing module 454. In the closed configuration, a portion of the housing 455 of the cell processing module 454 is positioned underneath the top plate 458, and a portion of the cell culture container 452 is positioned under the top plate 458. In particular, when the moveable rack 470 is in the closed configuration, the port 453 of the cell culture container 452, the flow coupler 476 of the cell processing module 454, and at least a portion of the actuation assembly 462 (e.g., the flow path, defined by for example, the needle) can be aligned to facilitate movement of the contents from the cell culture container 452 into the cell processing module 454 (e.g., to be processed). In addition, when the moveable rack 470 is in the closed configuration, a flow path of the fluid handling device 450 can be aligned with a flow path of the cell processing module 454. For example, a flow path of the fluid handling device 450 (e.g., for a pump, such as a syringe pump) can be aligned and subsequently brought into fluid communication with the flow path of the cell processing module 454 (e.g., by opening a valve) that is in fluid communication with a volume of fluid in a cell culture container or a centrifuge container. In some embodiments, when the moveable rack 470 is in a closed configuration, one or more multi-position valves of the cell processing module 454 can be brought into mechanical contact with one or more actuators (e.g., motors, and in particular a gear coupled to a shaft of the motor). When in contact, the one or more actuators can adjust the position of the one or more valves (e.g., by using a computing device) thereby adjusting flow paths within the cell processing module 454. In this way, the fluid handling device 450 can advantageously house the one or more actuators (and corresponding electrical connections as appropriate), rather than the cell processing module (and others), which prevents the need to connect (e.g., electrically, fluidly, etc.) the one or more actuators to the fluid handling device 450. Thus, a more automated approach can be established because a user does not have to manually disconnect and connect the actuators to the fluid handling device 450 for each different cell processing module. In addition, the actuators (e.g., including a motor) do not have to be disposed of when the cell processing module is disposed.

Referring back to FIG. 17, the fluid handling device 450 can include position sensors 478, 480, each of which can be positioned on an opposing end of the housing 456 of the fluid handling device 450. The position sensors 478, 480 can each be configured to sense a position of the moveable rack 470 and the components positioned thereon. The position sensors 478, 480 can be implemented in different ways. For example, the position sensors 478, 480 can be quadrature encoders, Hall-effect sensors, etc. In other cases, and as illustrated in FIG. 17, the position sensors 478, 480 can be optical sensors, each of which can include a light source configured to emit light towards the optical sensor. In addition to the optical sensors 478, 480, the fluid handling device 450, and in particular the moveable rack 470 of the fluid handling device 450, can include protrusions 482, 484, 486, 488 that are configured to interrupt an optical sensor from receiving light thereby indicating that the moveable rack 470 is at a particular position. For example, a computing device (e.g., of the fluid handling device 450) can cause the moveable rack 470 to move (e.g., by activating the actuator 468), and as the moveable rack 470 moves from the open configuration and to the closed configuration, each protrusion 482, 484, 486, 488 moves past the position sensor 480 (e.g., that is implemented as an optical sensor), which can be sensed by the position sensor 480. Then, a computing device can determine the position of the moveable rack 470, based on the number of occurrences of failing to receive light. In some cases, when the moveable rack 470 is in the open configuration, each position sensor 478, 480 can be unobstructed (e.g., not fully blocked by a protrusion). Conversely, when the moveable rack 470 is in the closed configuration, each position sensor 478, 480 can be obstructed (e.g., partially blocked by a protrusion). In this way, a computing device can determine that the moveable rack 470 is in the open configuration based on the computing device receiving an indication from each sensor 478, 480 that each sensor 478, 480 is not obstructed by a protrusion, while a computing device can determine that the moveable rack 470 is in the closed configuration, based on the computing device receiving an indication from each sensor 478, 480 that each sensor is obstructed by a protrusion.

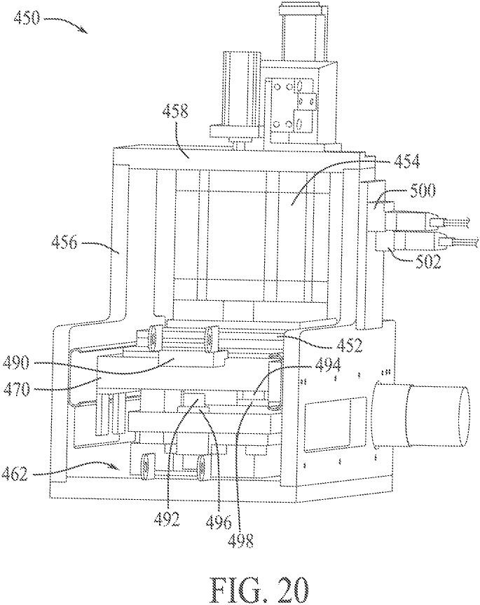

FIG. 20 shows a rear perspective view of the fluid handling device 450 with the moveable rack 470 in the closed configuration and supporting the cell culture container 452, and the cell processing module 454. The clamping assembly 462 can be configured to lift a tray 490 that is received on the moveable rack 470 (with the components situated thereon) upwardly until the housing 455 of the cell processing module 454 contacts the top plate 458, and downwardly until the tray 490 rests back onto the moveable rack 470. For example, the tray 490 can support both the cell culture container 452 and the cell processing module 454, and can be supported by the moveably rack 470. In particular, the tray 490 can be received within a recess of the moveable rack 470, which can ensure that the relative position between the tray 490 (and the components thereon) and the moveable rack 470 are consistent. In some embodiments, the clamping assembly 462 can include actuators 492, 494, which can be positioned on opposing sides of the actuation assembly 466 to contact opposing sides of the tray 490. Each actuator 492, 494 can include a piston that is received through a respective guide bushing 496, 498 to ensure proper extension (and retraction) of each piston and thus corresponding lifting (and lowering) of the tray 490. In some embodiments, each actuator 492, 494 can be pneumatic actuators, which are drivable by opening and closing a valve 500 that is in fluid communication with a pneumatic fluid source (e.g., air). In this way, opening of the valve 500 (e.g., by a computing device) can allow fluid to flow into each of the actuators 492, 494 thereby extending the piston of each actuator 492, 494 and raising the tray 490 from the rack 470 until the housing 455 of the cell processing module 454 contacts the top plate 458. Correspondingly, opening of the valve 500 to atmosphere (e.g., using a computing device) can allow fluid to flow along a flow path from each actuator 492, 494, through the valve 500, and into atmosphere, thereby lowering each piston of each actuator 492, 494, and thus lowering the tray 490 until the tray 490 contacts the moveable rack 470.

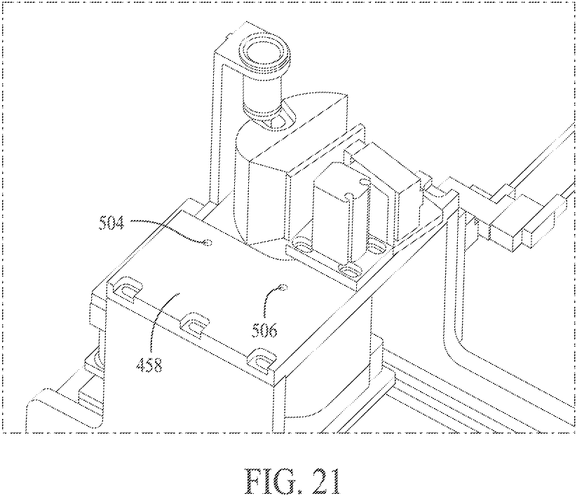

In some embodiments, the clamping assembly 462 can not only secure the cell culture container 452 and the cell processing module 454, but the clamping assembly 462 can also fluidly connect components (e.g., flow paths of components) and electrically connect components. For example, as shown in FIG. 21, the top plate 458 can include ports 504, 506, which can be in fluid communication with components of the fluid handling device 450 (e.g., a pump) can each be brought into fluid communication with a cell processing consumable (e.g., a pressure chamber, a cell culture container, etc.), a pump, etc., when the housing 455 of the cell processing module 454 is forced against and contacts the top plate 458. For example, each port 504, 506 can include a gasket that engages with a corresponding inlet (or outlet) of the fluid path of the cell processing module 454 when the housing 455 contacts the top plate 458 to fluidically isolate each fluid path of the cell processing module 454. In some embodiments, each port 504, 506 can include an actuatable valve (e.g., a solenoid valve, a pinch valve, etc.) to allow (or block) fluid communication through the respective port 504, 506. In other embodiments, each port 504, 506 can be engaged by a flow coupler (e.g., by extending the actuator of the flow coupler until the flow coupler is in engagement with the port).

In some embodiments, including when the cell processing module 454 includes one or more electrodes (e.g., the cell processing module 454 being configured to perform electroporation on cells), the one or more electrodes can electrically connect to an electrical connector of the fluid handling device 450 when the housing 455 of the cell processing module 454 is brought into contact with the top plate 458. In this way, the fluid handling device 450 can provide power (e.g., a voltage) to the one or more electrodes of the cell processing module 454 to perform the electroporation in a sterile manner. In other words, because the one or more electrodes are positioned within the cell processing module 454, there is less risk to contamination if, for example, the one or more electrodes were located at the fluid handling device 450. In other words, the cell processing module 454 can be disposed of and the fluid handling device 450 can be reused with other cell processing modules without the risk of contaminating the materials (e.g., reagents) of the new cell processing module.

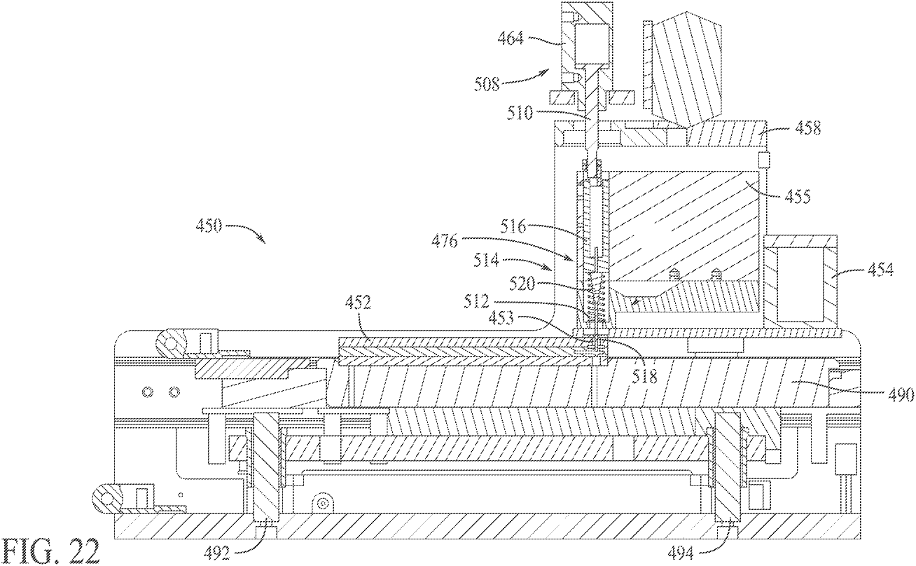

FIG. 22 shows a side cross-sectional view of the fluid handling device 450 with the tray 490 supporting the cell culture container 452, and the cell processing module 454, and with the moveable rack 470 in the closed configuration. As shown in FIG. 22, the port 453 of the cell culture container 452, the flow coupler 476 (e.g., the flow path of the flow coupler 476), and the actuation assembly 464 are aligned. In particular, the port 453, the flow path of the flow coupler 476, and an actuator of the actuation assembly 464 are aligned. In addition, the actuators 492, 494 have been extended to raise the tray 490 until the housing 455 of the cell processing module 454 contacts the top plate 458. In some embodiments, once these components have been aligned, the actuation assembly 464 can be activated to puncture the septum of the port 453 of the cell culture container 452 to access the interior volume of the cell culture container 452. For example, the actuation assembly 464 can include an actuator 508 with a piston 510 that can extend and retract, while the flow coupler 476 can be structured similarly to the other flow couplers described herein (e.g., the flow coupler 408) and thus the flow coupler 476 can include a spring 512, and a needle assembly 514 including a base 516, a needle 518, and a flow path 520 through the base 516 and the needle 518. When a computing device activates the actuator 508 (e.g., by opening a pneumatic valve 502 to drive pneumatic fluid into the actuator 508), the piston 510 extends to contact the base 516 thereby driving the needle 518 through the septum of the port 453 of the cell culture container 452. In this way, the flow path 520 of the needle assembly 514 is brought into fluid communication with the interior volume of the cell culture container 452 (e.g., that includes cells, culture media, etc.), and the cells located within the interior volume can be removed from the cell culture container 452 via the flow path 520.

In some embodiments, after the piston 510 extends, the actuator 508 can cause the piston 510 to retract. When this occurs, because the base 516 of the needle assembly 514 biases the spring 512 during extension of the piston 510, retraction of the piston 510 can cause the needle 518 to retract via unloading of the spring 512 onto the base 516. In some cases, retraction of the needle 518 can cause the needle 518 to be removed from the port 453 of the cell culture container 452. In some configurations, with the cell processing module 454 including the flow coupler 476 that is actuatable by the actuator 508 of the fluid handling device 450, the fluid handling device 450 can be reused for other cell processing modules without fear of contamination. In other words, because the flow couplers are not being reused (e.g., are not located within the fluid handling device 450), but rather are disposed of after each use with the corresponding cell processing module, the flow couplers and in particular the needle of a flow coupler does not have to be thoroughly cleaned before usage with different cell processing modules.

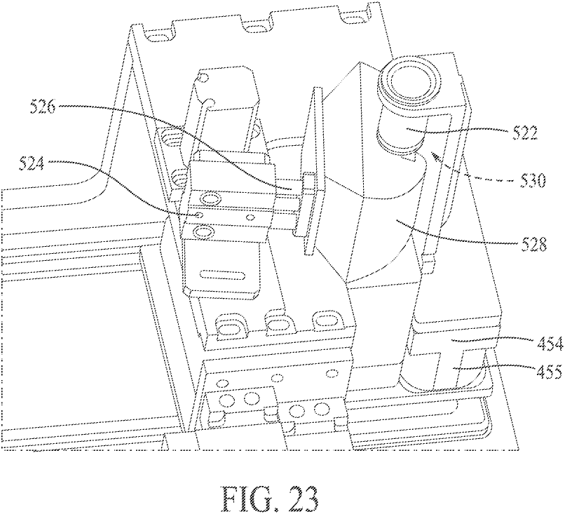

In some embodiments, the magnet assembly 466 of the fluid handling device 450 can be used for cell processing modules that facilitate cell isolation, cell debeading, etc. For example, as shown in FIG. 23, the cell processing module 454 can include a magnet chamber 522 for cell debeading, cell isolation, etc. In some configurations, the magnet chamber 522 can be a magnetic column (e.g., containing magnetic binding agents, including resins). The magnet assembly 466 can include an actuator 524 having a piston 526, and a magnet 528 that is coupled to the piston 526, which has a recess 530. In some embodiments, the magnet 528 can be an electromagnet, which can be excited by a computing device of the fluid handling device 450 (e.g., by driving current through the electromagnetic), while in other cases, the magnet 528 can be a permanent magnet. In some cases, the magnet 528 being a permanent magnet can be advantageous in that the permanent magnet can generate higher amounts of magnetic flux (e.g., as compared to an electromagnet of similar size), and does not require relatively high driving currents required by the electromagnet.

As shown in FIG. 23, the actuator 524 has extended the piston 526 (e.g., by opening a pneumatic valve) thereby moving the magnet 528 until, for example, the magnet chamber 522 is received into the recess 530 of the magnet 528. In some embodiments, the actuator 524 can extend the piston 526 until the magnet chamber 522 contacts the magnet 528 (e.g., while the magnet chamber 522 is situated within the recess 530 of the magnet 528). With the selective movement of the magnet 528, the magnet 528 can be extended when the magnet 528 is to be used for cell debeading, cell isolation, but can be retracted to create additional space when the magnet 528 is not needed for the cell process provided by the cell processing module.

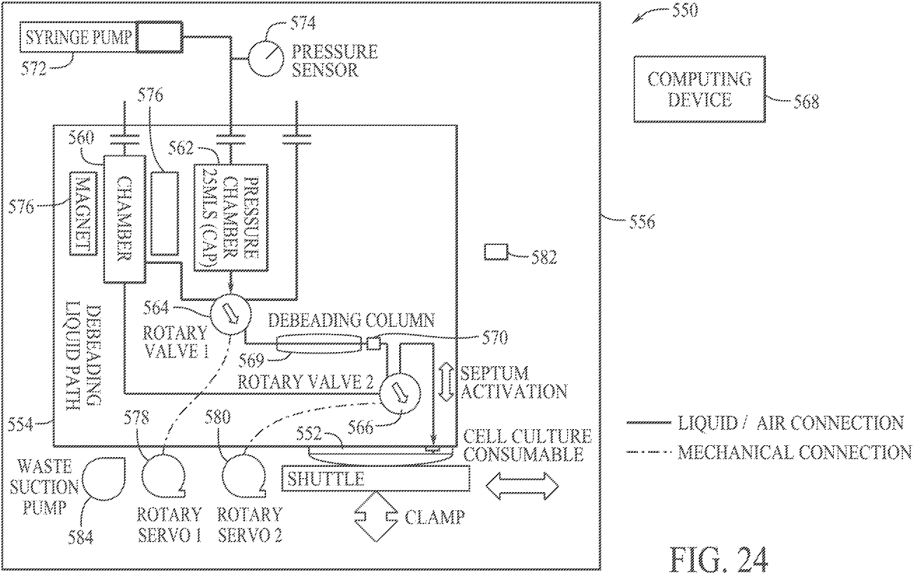

FIG. 24 shows a schematic illustration of a cell processing system 550, which can be a specific implementation of the cell processing systems described herein (e.g., the cell processing system 100). In some embodiments, the cell processing system 550 can be configured to perform a debeading process on cells flowing through the cell processing system 550. The cell processing system 550 can include a cell culture container 552, a cell processing module 554, a fluid handling device 556, and a computing device 568 in communication with the fluid handling device 556 (e.g., to control components of the fluid handling device 556). In some cases, the cell culture container 552, the cell processing module 554, and the fluid handling device 556 can be implemented in a similar manner as components described herein with similar corresponding names.

As shown in FIG. 24, the cell processing module 554 can include a magnet chamber 560, a pressure chamber 562, multi-position valves 564, 566, a debeading column 569, and a pump 570. The fluid handling device 556 can include a pump 572, a pressure sensor 574 in communication with the pump 572 (e.g., at the outlet of the pump 572), a magnet 576, motors 578, 580, 582, a waste pump 584. The magnet chamber 560 can be positioned so that the magnet 576 at least partially surrounds the magnet chamber 560. In this way, magnetic beads (including components coupled to the bead such as antibodies with cells coupled thereto), can be forced against the inner side of the wall of the magnet chamber 560. The pressure chamber 562 can be in fluid communication with the pump 572, which can be a syringe pump, and can function as a storage chamber for storing liquid (having cells).

Similarly to the discussion of the fluid handling device 450 above, the multi-position valves 564, 566 can each change the flow path of fluid flow within the cell processing module 554, and are each mechanically engaged with a respective motor 578, 580. In this way, activation of the motors 578, 580 can adjust the flow paths within the cell processing module 554 in a relatively sterile manner (e.g., because the inner flow paths of the multi-position valves 564, 566 are isolated from the motors 578, 580). In some embodiments, and as illustrated in FIG. 24, each multi-position valve 564, 566 can be a rotary valve. The debeading column 569 can include magnetic binding agents that attract and bind to magnetic beads forced through the debeading column 569. In some embodiments, the pump 570 can be positioned between the debeading column 569 and the valve 566, and can be mechanically engaged with the motor 582 in a similar manner as the engagement between the motors 578, 580 and the respective valves 564, 566. In this way, the pump 570 can be fluidically isolated from the motor 582, but the motor 582 can drive pumping (and the pumping direction) of the pump 570. In some configurations, the pump 570 can be a two-way pump, so that the pump 570 can, for example, drive fluid through the debeading column 569 in both flow directions. While the magnet 576 has been described as being part of the fluid handling device 556, which can include the selective engagement between the magnet 576 and the magnet chamber 560 (e.g., in a similar manner as the magnet 528 and the magnet chamber 522 described above), in alternative configurations, the cell processing module 554 can include the magnet 576. In this case, for example, the magnet 576 can be coupled to a housing of the cell processing module 554 and can be fixed relative to the magnet chamber 560. In some embodiments, the waste pump 584 can include a valve to selectively allow and block fluid communication between a flow path of a cell processing module. In some embodiments, some cell processing models including the cell processing module 554 do not include a waste chamber for storing waste fluid from a flow path of the cell processing module.

In some embodiments, the computing device 568 can be in communication with some or all of the components of the cell processing system 550, as appropriate. For example, the computing device 568 can be in communication (and can control) the pump 572, the pressure sensor 574, the motors 578, 580, 582, the waste pump 584, and other components described herein (e.g., actuators, including those that control a flow coupler).

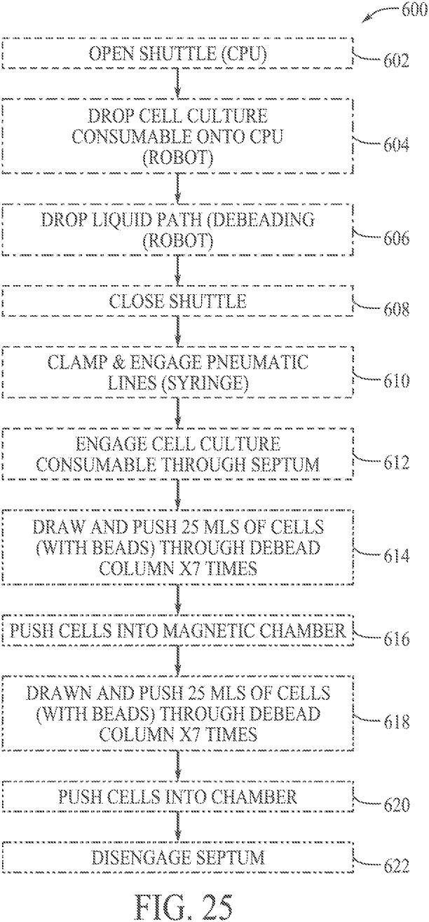

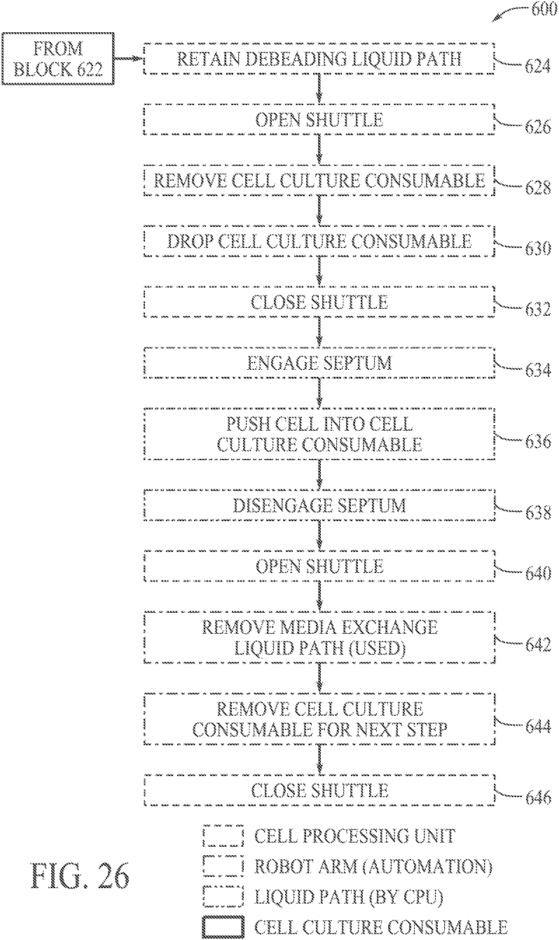

FIGS. 25 and 26 collectively show a flowchart of a process 600 for performing a cell debeading process. In some embodiments, the process 600 can be implemented using any of the cell processing systems (and corresponding components), but will be described mainly with reference to the cell processing system 550. Similarly, some or all blocks of the process 600 can be implemented using one or more computing devices, as appropriate, but will reference mainly the corresponding computing device 568 of the cell processing system 550.

At 602, the process 600 can include a computing device causing a shuttle assembly of a fluid handling device to open. For example, this can include a computing device causing an actuator to move a moveable rack of the receptacle to an open configuration.

At 604, the process 600 can include a computing device placing a cell culture container (or centrifuge container) into the receptacle. In some cases, this can include a computing device causing a robot arm to pick up a cell culture container, and place the cell culture container onto the moveable rack (e.g., the tray that is supported by the moveable rack). In some cases, this can include engaging the cell culture container (e.g., the cell culture container 452) with an engagement feature of the moveable rack (or the tray supported by the moveable rack).

At 606, the process 600 can include a computing device placing a cell processing module (e.g., a debeading cell processing module) into the receptacle. For example, this can include a computing device causing a robot arm to pick up the cell processing module and place the cell processing module onto the moveable rack. In some cases, this can include engaging the housing of the cell processing module with an engagement feature of the moveable rack (or the tray supported by the moveable rack). In some embodiments, this can include aligning a port of the cell culture container with a flow path of a flow coupler of the cell processing module (e.g., when the cell culture container is placed on the moveable rack, and when the cell processing module is placed on the moveable rack). In some configurations, this can include engaging (and aligning) each motor (e.g., the motors 578, 580) with a corresponding multi-position valve (e.g., the multi-position valve 564, 566) of the fluid handling device. In some configurations, this can include engaging a motor (e.g., the motor 582) with a pump (e.g., the pump 570) of the fluid handling device. In some cases, each motor can be positioned on the moveable rack of the fluid handling device.

At 608, the process 600 can include a computing device causing a shuttle assembly of a fluid handling device to close. For example, this can include a computing device causing an actuator to move a moveable rack of the receptacle to a closed configuration. In some configurations, a computing device can receive, from one or more position sensors, position sensor data, and can determine that the moveable rack of the receptacle is in the closed configuration. In some embodiments, this can include aligning the flow coupler with an actuator of an actuation assembly of the receptacle.

At 610, the process 600 can include a computing device clamping the cell processing module to the receptacle. For example, this can include a computing device causing one or more actuators (e.g., of the receptacle) to move the cell processing module (and the cell culture container) into contact with the housing of the receptacle. As a more specific example, this can include a computing device causing one or more actuators to lift a tray that supports the cell processing module and the cell culture container, off the moveable rack and until the cell processing module contacts a top plate of the receptacle. In some cases, this can include a computing device causing one or more ports of the receptacle (e.g., the top plate of the receptacle) to fluidly connect with one or more flow paths of the cell processing module. For example, when the cell processing module contacts the top plate of the receptacle, a first port of the receptacle aligns and fluidly connects with a first flow path of the cell processing module, and a second port of the receptacle aligns and fluidly connects with a second flow path of the cell processing module. In some cases, when a port fluidly connects with a flow path, the port and the flow path can be sealed from the ambient environment.

At 612, the process 600 can include a computing device fluidly connecting a flow path of the flow coupler of the cell processing module with an interior volume of a cell culture container (e.g., by actuating an actuator). In some cases, this can include a computing device causing an actuator to extend a piston to drive a needle (e.g., downwardly) through a septum of a port of the cell culture container thereby fluidly connecting the flow path of the needle with the interior volume of the cell culture container. In some embodiments, this can include, when extending the piston of the actuator, mechanically biasing the flow coupler (e.g., the needle assembly of the flow coupler) using a spring.

At 614, the process 600 can include a computing device drawing a volume of liquid from the interior volume of the cell culture container. In some cases, this can include a computing device causing a motor to adjust a position of a multi-position valve to bring a pump (e.g., the pump 570) in fluid communication with the port of the cell culture container, and causing the pump to draw the volume of liquid from the interior volume of the cell culture container, and through the flow path of the flow coupler. In some cases, the volume of liquid can be substantially (i.e., deviating by less than 10%) 25 mL.

In some embodiments, the block 614 of the process 600 can include a computing device directing the volume of liquid through a debeading column (of the cell processing module) in a first direction, and directing the volume of liquid through the debeading column in a second direction opposite the first direction, each of which can be completed one or more times (e.g., seven times). For example, a computing device can cause the pump to direct the volume of liquid through the debeading column in the first direction. In some cases, a computing device can cause a multi-position valve (e.g., the multi-position valve 564) to block fluid flow past the multi-position valve. In addition, the entire volume of liquid, when flowing in the first direction, can flow through past the debeading column. In this way, the entire volume of liquid (e.g., 25 mL) is exposed to a greater surface area of the debeading column (and thus a larger number of magnetic binding agents). Then, a computing device, after causing the multi-position valve to block fluid flow past the multi-position valve and to the port of the cell culture container, can cause the pump to direct the volume of liquid through the debeading column in a second direction. Similarly, the entire volume of liquid, when flowing in the second direction, can flow past the debeading column. This process (passing the volume through the debeading column in both directions) can be repeated a number of times (e.g., seven), with the more times the liquid is passed through the greater likelihood that the magnetic beads bind to the debeading column. As the volume of fluid flows through the debeading column, magnetic beads located within the volume, some of which have antibodies attached thereto (e.g., which are coupled to cells, via for example, interactions between the fragment antigen-binding and a cell) are attracted and bound to the debeading column. In this way, this process can remove undesirable cells (e.g., those that are captured by the debeading column).

At 616, the process 600 can include a computing device directing the volume of liquid into a magnet chamber of the cell processing module. For example, this can include a computing device causing a motor to move a multi-position valve to allow flow between the debeading column and the magnet chamber, and causing a pump to direct the volume of liquid into the magnet chamber. In some embodiments, a computing device can extend an actuator to move a magnet into engagement with the magnet chamber, or can provide power to the magnet (e.g., that is an electromagnet). Regardless of the configuration, as the volume of liquid is directed into the magnet chamber, the magnetic flux provided by the magnet attracts magnetic beads (e.g., leftover magnetic beads with no cells coupled thereto, or magnetic beads with cells coupled thereto) within the volume of liquid against a wall of the magnet chamber. In some embodiments, after the volume of liquid is situated within the magnet chamber, the volume of liquid can be kept within the magnet chamber for a period of time (e.g., fifteen minutes). In this way, waiting the period of time can ensure that the magnet appropriately attracts the beads.

At 618, the process 600 can include a computing device directing the volume of liquid from the magnet chamber and through the column, in both the first and second directions, one or more times (e.g., seven times), which can be similar to the block 614.

At 620, the process 600 can include a computing device directing the volume of liquid into a storage chamber of the removable cell processing module. In some cases, this volume of liquid includes cells of a first type that are not magnetically attracted by the magnet or the debeading column, different from cells of a second type that were magnetically attracted by the magnet, and the debeading column (and trapped to the component). In this way, the chamber largely (and ideally) receives only cells of the first type. In some embodiments, this can include a computing device causing a motor to adjust the position of a multi-position valve to allow fluid communication between the magnet chamber (or magnet column) and the storage chamber (of the cell processing module), and causing a pump (e.g., the pump 572) to drive the volume of fluid from a flow path of the cell processing module (e.g., the magnet chamber, the debeading column, etc.) and into the storage container.

At 622, the process 600 can include a computing device fluidly disconnecting the flow path of the flow coupler from the interior volume of the cell culture container. In some cases, this can include a computing device causing an actuator to retract a piston, which correspondingly causes a spring to retract the flow coupler thereby retracting the needle (e.g., upwardly) back through and out of the septum of the cell culture container.

At 624, the process 600 can include a computing device causing the volume of liquid to be retained within the storage chamber of the cell processing module. For example, this can include a computing device closing a valve at an inlet (or outlet) of the storage chamber to fluidly isolate the storage chamber from a flow path of the cell processing module. In some cases, the storage chamber can be a pressure chamber (e.g., that includes cell media positioned therein). In some embodiments, this can include retaining the cell processing module for use with other cell culture containers (e.g., described in more detail below).

At 626, the process 600 can include a computing device causing a shuttle assembly of a fluid handling device to open, which can be similar to the block 602.

At 628, the process 600 can include a computing device removing the cell culture container from the receptacle, which can be the opposite as the block 604. For example, this can include a computing device causing a robot arm to pick up the cell culture container and remove the cell culture container from the receptacle (e.g., the moveable tray). In some cases, this can include disposing the cell culture container (e.g., the robot arm placing the cell culture container in a waste receptacle).

At 630, the process 600 can include a computing device placing a different cell culture container (e.g., a new cell culture container) into the receptacle, which can be similar to the block 604.

At 632, the process 600 can include a computing device causing a shuttle assembly of a fluid handling device to close, which can be similar to the block 608. In some embodiments, the process 600 can include clamping the different cell processing module to the receptacle, which can be similar to the block 610.

At 634, the process 600 can include a computing device fluidly connecting the flow path of the flow coupler of the cell processing module with an interior volume of the different cell culture container, which can be similar to the block 612.

At 636, the process 600 can include a computing device directing the volume of liquid (that includes cells of first type) from the storage chamber and into the interior volume of the different cell culture container. In some cases, this can include a computing device causing a motor to adjust the position of a multi-position valve to allow fluid communication between the storage chamber and the interior volume of the cell culture container, and causing a pump to drive the volume of liquid from the storage chamber, through the flow path of the flow coupler, and into the interior volume of the cell culture container.

At 638, the process 600 can include a computing device fluidly disconnecting the flow path of the flow coupler of the cell processing module from the interior volume of the cell culture container, which can be similar to the block 622.

At 640, the process 600 can include a computing device causing a shuttle assembly of a fluid handling device to open, which can be similar to the blocks 602, 626.

At 642, the process 600 can include a computing device removing the cell processing module from the receptacle, which can be opposite as the block 606. For example, this can include a computing device causing a robot arm to pick up the cell processing module and remove the cell processing module from the receptacle (e.g., the moveable tray). In some cases, this can include disposing the cell processing module (e.g., the robot arm placing the cell processing module in a waste receptacle).

At 644, the process 600 can include a computing device removing the different cell culture container (e.g., which can now include the cells of the first type) from the receptacle. In some cases, this can include a robot arm picking up the different cell culture container and placing the different cell culture container into an incubator.

At 646, the process 600 can include a computing device causing a shuttle assembly of the receptacle to close, which can be similar to the blocks 608, 632.

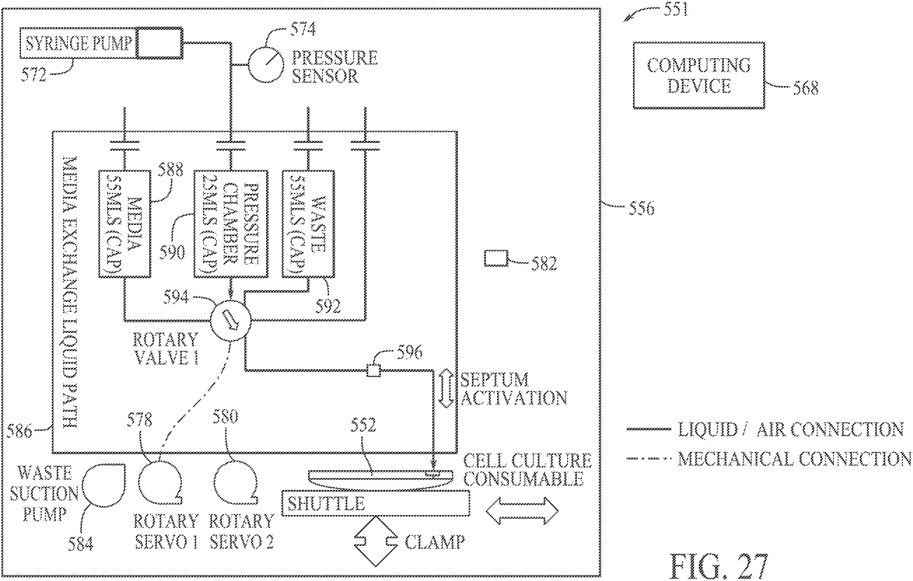

FIG. 27 shows a schematic illustration of a cell processing system 551, which can be a specific implementation of the cell processing systems described herein (e.g., the cell processing system 100). In some embodiments, the cell processing system 551 can be configured to perform a cell media exchange for a cell culture container. The cell processing system 551 can include the cell culture container 552, the fluid handling device 556, the computing device 568 in communication with the fluid handling device 556 (e.g., to control components of the fluid handling device 556), and a cell processing module 586. In some cases, the cell culture container 552, the cell processing module 586, and the fluid handling device 556 can be implemented in a similar manner as components described herein with similar corresponding names.

As shown in FIG. 27, the cell processing module 586 can include a cell media chamber 588, a pressure chamber 590, a waste chamber 592, a multi-position valve 594, and a pump 596. The cell media chamber 588 can hold a particular volume of cell media, and the waste chamber 592 can store a particular volume of waste liquid (e.g., spent cell media). In some embodiments, the interior volume of the cell media chamber 588 and the waste chamber 592 can be substantially the same. Similarly to the cell processing module 554, when the cell processing module 586 is engaged with the fluid handling device 556, the motor 578 can engage the multi-position valve 594 (e.g., to adjust the position of the multi-position valve 594), while the motor 582 can engage the pump 596 (e.g., to drive fluid flow through the pump), which is situated between the multi-position valve 594 and the flow path of the flow coupler of the cell processing module 586. In some configurations, similarly to the pump 570, the pump 596 can be a two-way pump.

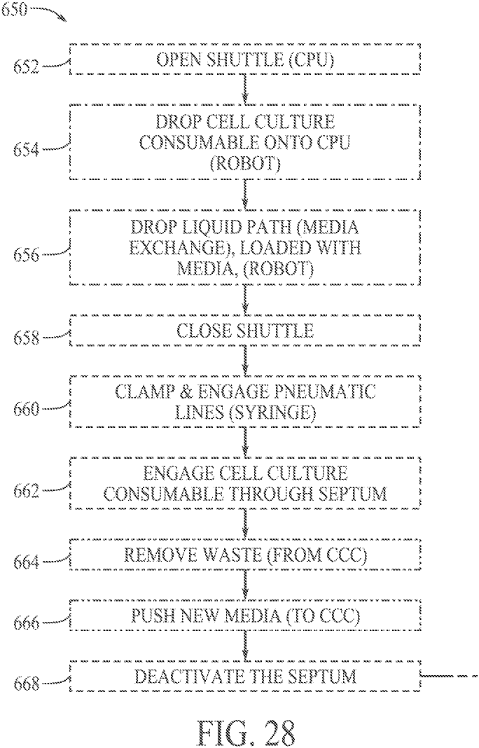

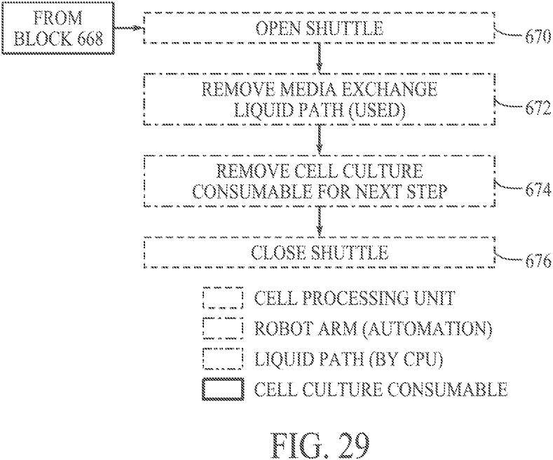

FIGS. 28 and 29 collectively show a flowchart of a process 650 for performing a cell debeading process. In some embodiments, the process 650 can be implemented using any of the cell processing systems (and corresponding components), but will be described mainly with reference to the cell processing system 551. Similarly, some or all blocks of the process 650 can be implemented using one or more computing devices, as appropriate, but will reference mainly the corresponding computing device 568 of the cell processing system 551.

At 652, the process 650 can include a computing device causing a shuttle assembly of a fluid handling device to open, which can be similar to the block 602 of the process 600. At 654, the process 650 can include a computing device placing a cell culture container into the receptacle, which can be similar to the block 604 of the process 600. At 656, the process 650 can include a computing device placing a cell processing module (e.g., a cell culture cell processing module) into the receptacle, which can be similar to the block 606 of the process 600. At 658, the process 650 can include a computing device causing a shuttle assembly of a fluid handling device to close, which can be similar to the block 608 of the process 600. At 660, the process 650 can include a computing device clamping the cell processing module to the receptacle, and causing one or more ports of the receptacle to fluidly connect with one or more flow paths of the cell processing module (e.g., when the housing of the cell processing module contacts a top plate of the receptacle), each of which can be similar to the block 610 of the process 600. At 662, the process 650 can include a computing device fluidly connecting a flow path of a flow coupler of the cell processing module with an interior volume of the cell culture container, which can be similar to the block 612 of the process 600.

At 664, the process 650 can include a computing device removing waste from the interior volume of the cell culture container and directing the waste into a waste chamber of the cell processing module. For example, this can include a computing device causing a motor (e.g., the motor 578) to rotate a multi-position valve (e.g., the multi-position valve 594) to bring the interior volume of the cell culture container into fluid communication with the waste chamber of the cell processing module. Then, a computing device can cause a pump (e.g., by activating a motor) to direct fluid (e.g., that is substantially devoid of cells) out from the interior volume of the cell culture container, through the flow path of the flow coupler of the cell processing module, through the multi-position valve, and into the waste chamber. In some cases, the computing device can cause the pump to direct a particular amount of liquid from the interior volume of the cell culture container into the waste chamber. For example, the particular volume can be substantially 55 mL. In addition, the particular amount of liquid can be substantially free of cells, which can be completed by, for example, by centrifuging the cells into a pellet prior to drawing liquid out of the interior volume of the cell culture container.

At 666, the process 650 can include a computing device directing fresh cell media from a cell media chamber (e.g., the cell media chamber 588) into the interior volume of the cell culture container. For example, this can include a computing device causing a motor (e.g., the motor 578) to rotate a multi-position valve (e.g., the multi-position valve 594) to bring the interior volume of the cell culture container into fluid communication with the cell media chamber of the cell processing module. Then, a computing device can cause a pump (e.g., the pump 596) to direct an amount of fresh cell culture media from the cell media chamber, through the multi-position valve, through the flow path of the flow coupler, and into the interior volume of the cell culture container. In some cases, the amount of fresh cell culture media can substantially corresponding to amount of liquid previously removed from the interior volume of the cell culture container.

At 668, the process 650 can include a computing device fluidly disconnecting the flow path of the flow coupler from the interior volume of the cell culture container, which can be similar to the block 622 of the process 600.

At 670, the process 650 can include a computing device causing a shuttle assembly of a fluid handling device to open, which can be similar to the block 602 of the process 600. At 672, the process 650 can include a computing device removing the cell processing module from the receptacle, which can be similar to the block 642 of the process 600. At 674, the process 650 can include a computing device removing the cell culture container from the receptacle, which can be similar to the block 644 of the process 600. At 676, the process 650 can include a computing device causing a shuttle assembly of a fluid handling device to close, which can be similar to the block 608 of the process 600.

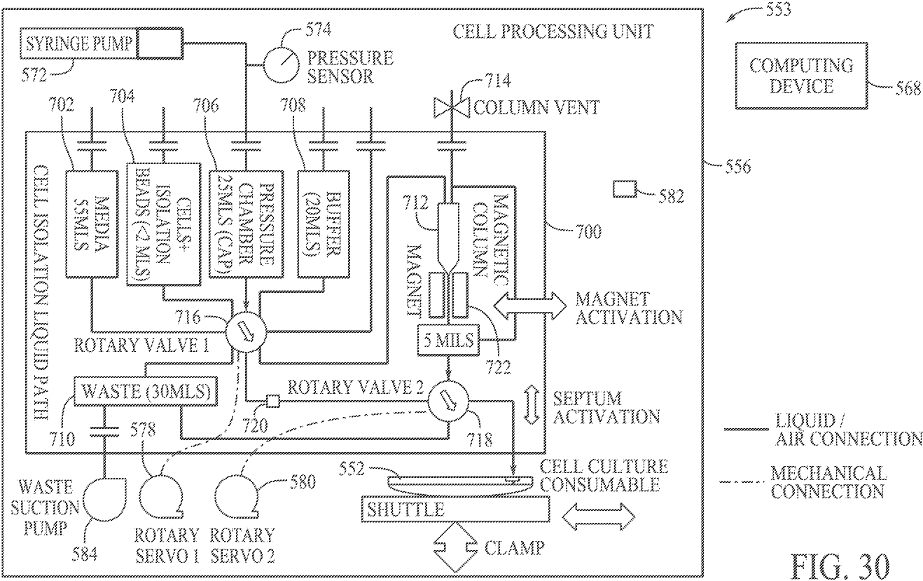

FIG. 30 shows a schematic illustration of a cell processing system 553, which can be a specific implementation of the cell processing systems described herein (e.g., the cell processing system 100). In some embodiments, the cell processing system 553 can be configured to perform a cell media exchange for a cell culture container. The cell processing system 553 can include the cell culture container 552, the fluid handling device 556, the computing device 568 in communication with the fluid handling device 556 (e.g., to control components of the fluid handling device 556), and a cell processing module 700. In some cases, the cell culture container 552, the cell processing module 700, and the fluid handling device 556 can be implemented in a similar manner as components described herein with similar corresponding names.

As shown in FIG. 30, the cell processing module 700 can include a cell media chamber 702, a cell chamber 704, a pressure chamber 706, a buffer chamber 708, a waste chamber 710, a magnetic column 712, a vent 714, multi-position valves 716, 718, and a pump 720. The cell media chamber 702 can store a volume of cell media, the cell chamber 704 can store cells of multiple types to be sorted with a first type of cell having one or more magnetic beads coupled thereto, the buffer chamber 708 can store a buffer solution to elute off components bound to the magnetic column 712, and the waste chamber 710 can store a volume of waste (e.g., liquid from washing the magnetic column 712). In some cases, the vent 714 can relieve gas pressure for the magnetic column 712. Similarly to the cell processing module 554, when the cell processing module 700 is engaged with the fluid handling device 556, the motor 578 can engage the multi-position valve 716 (e.g., to adjust the position of the multi-position valve 716), the motor 580 can engage the multi-position valve 718 (e.g., to adjust the positon of the multi-position valve 718), and the motor 582 can engage the pump 720 (e.g., to drive fluid flow through the pump), which can be situated between the multi-position valves 716, 718. In some configurations, the pump 720 can be a two-way pump.

In some embodiments, the fluid handling device 556 can include a magnet 722 that can selectively be brought into and out of alignment with the magnetic column 712. For example, a computing device can cause an actuator of the receptacle can extend the magnet 722 so that the magnetic column 712 is received in magnet 722 (e.g., a recess of the magnet 722), and can similarly cause the actuator to retract the magnet 722 so that the magnetic column 7112 is removed from the magnet 722.

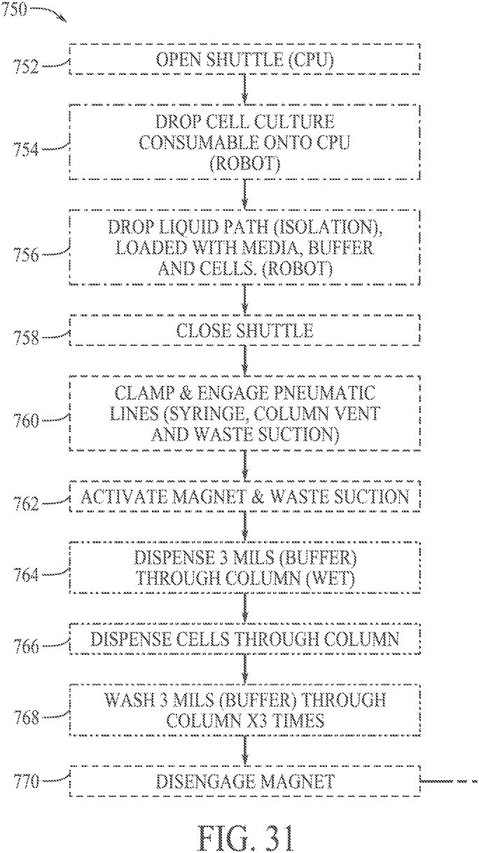

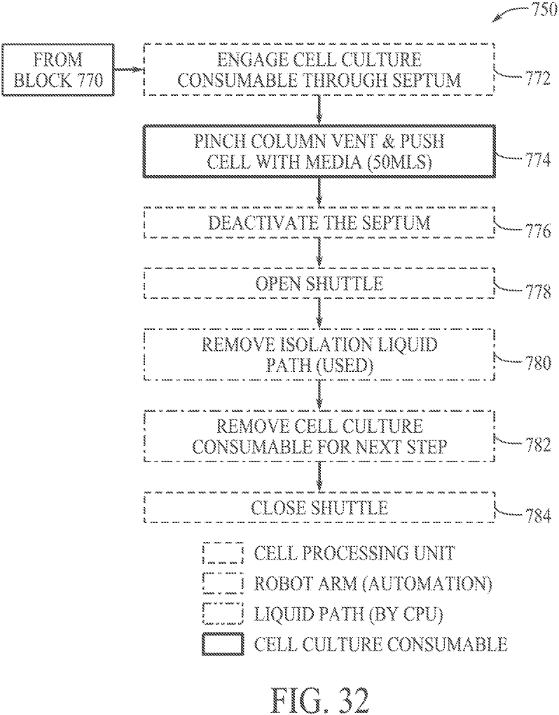

FIGS. 31 and 32 collectively show a flowchart of a process 750 for performing a cell isolation process. In some embodiments, the process 750 can be implemented using any of the cell processing systems (and corresponding components), but will be described mainly with reference to the cell processing system 553. Similarly, some or all blocks of the process 750 can be implemented using one or more computing devices, as appropriate, but will reference mainly the corresponding computing device 568 of the cell processing system 553.

At 752, the process 750 can include a computing device causing a shuttle assembly of a fluid handling device to open, which can be similar to the block 602 of the process 600. At 754, the process 750 can include a computing device placing a cell culture container into the receptacle, which can be similar to the block 604 of the process 600. At 754, the process 750 can include a computing device placing a cell processing module (e.g., a cell isolation cell processing module) into the receptacle, which can be similar to the block 606 of the process 600. At 758, the process 750 can include a computing device causing a shuttle assembly of a fluid handling device to close, which can be similar to the block 608 of the process 600.

At 760, the process 750 can include a computing device clamping the cell processing module to the receptacle, and causing one or more ports of the receptacle to fluidly connect with one or more flow paths of the cell processing module (e.g., when the housing of the cell processing module contacts a top plate of the receptacle), each of which can be similar to the block 610 of the process 600. For example, this can include a computing device causing a pump port to fluidly connect to a first flow path of the cell processing module, a vent port to fluidly connect to a second flow path of the cell processing module, and a waste port to fluidly connect to a third flow path of the cell processing module.

At 762, the process 750 can include a computing device activating a magnet. In some cases, this can include a computing device causing an actuator to move a magnet into alignment with a magnetic column of the cell processing module.

At 764, the process 750 an include a computing device dispensing an amount of buffer through the magnetic column and into a waste chamber. For example, this can include a computing device causing a first multi-position valve to bring the buffer chamber (e.g., the buffer chamber 708) into fluid communication with the magnetic column, and causing a second multi-position valve to bring the magnetic column into fluid communication with the waste chamber. Then, an amount of buffer (e.g., 3 mL) can flow (e.g., from being pressurized within the buffer container, from gravity, from a pump, etc.) though the first multi-position valve, through the magnetic column (thereby washing any contents off the magnetic column), through the second multi-position valve, and into the waste chamber.

At 766, the process 750 can include a computing device dispensing the liquid including cells from the cell chamber (e.g., the cell chamber 704) and through the magnetic column 712. In some cases, this can include a computing device causing the first multi-position valve to bring the cell chamber into fluid communication with the magnetic column. Then, the liquid including cells from the cell culture container can flow (e.g., by gravity, a pump, etc.), through the multi-position valve, through the magnetic column, through the second multi-position valve, and into the waste chamber. As the liquid containing cells flows through the magnetic column, cells with magnetic beads coupled thereto are trapped in the magnetic column (e.g., by the magnetic binding agents, and the magnetic flux provided by the magnet).

At 768, the process 750 can include a computing device washing the magnetic column a number of times (e.g., one, two, three, etc.). For example, this can include a computing device causing the first multi-position valve to bring the buffer chamber into fluid communication with the magnetic column. Then, an amount of buffer can flow through the first multi-position valve, flow through the magnetic column, flow through the second multi-position valve, and flow into the waste chamber. This can occur a number of times (e.g., three times), by for example, a computing device causing the multi-position valve to block and resume fluid communication from the buffer chamber and to the magnetic column.

At 770, the process 750 can include a computing device deactivating the magnet. In some cases, this can include a computing device causing an actuator to move the magnet out of alignment with the magnetic column of the cell processing module.

At 772, the process 750 can include a computing device fluidly connecting a flow path of the flow coupler of the cell processing module with an interior volume of a cell culture container, which can be similar to the block 612 of the process 600.

At 774, the process 750 can include a computing device directing culture media through the magnetic column and into the cell culture container. For example, this can include a computing device causing the first multi-position valve to bring the cell media chamber into fluid communication with the magnetic column, and causing the second multi-position valve to bring the magnetic column into fluid communication with the interior volume of the cell culture container. Then, the cell culture media can flow from the cell culture container (e.g., by gravity, a pump, etc.), through the first multi-position valve, through the magnetic column, through the flow path of the flow coupler, and into the interior volume of the cell culture container. As the cell culture media flow through the magnetic chamber, cells previously bound are eluted off the magnetic column, and flow with the cell culture media ultimately being deposited into the interior volume of the cell culture container.

At 776, the process 750 can include a computing device fluidly disconnecting the flow path of the flow coupler from the interior volume of the cell culture container, which can be similar to the block 622 of the process 600.

At 778, the process 750 can include a computing device causing a shuttle assembly of a fluid handling device to open, which can be similar to the block 602. At 780, the process 750 can include a computing device removing the cell culture container from the receptacle, which can be similar to the block 644 of the process 600. At 782, the process 750 can include a computing device removing the cell processing module from the receptacle, which can be similar to the block 642 of the process 600. At 784, the process 750 can include a computing device causing a shuttle assembly of a fluid handling device to close, which can be similar to the block 608 of the process 600.

In some embodiments, a cell processing system as descried herein, can facilitate performing a centrifuge process on cells. For example, the centrifuge process can include removing the liquid (including cells) from a cell culture container and directing the liquid (including cells) into a centrifuge container (e.g., the centrifuge container 320), which can follow similar processes as those described in processes 600, 650, 750. Then, the centrifuge container can be placed (e.g., manually, or automatically using for example, a robot arm) into a swing bucket centrifuge. After the centrifuge process a cell pellet can form at a port of the centrifuge container, which can be extracted, or the supernatant liquid (including spent media) can be extracted from a port of the centrifuge container. In some cases, the cell pellet can be resuspended (e.g., via agitation by newly added cell media via introduction through one or more ports of the centrifuge container). In some configurations, after resuspending the cells can be grown in the centrifuge container, or alternatively, can be grown in a different cell culture container (e.g., following similar processes descried herein).

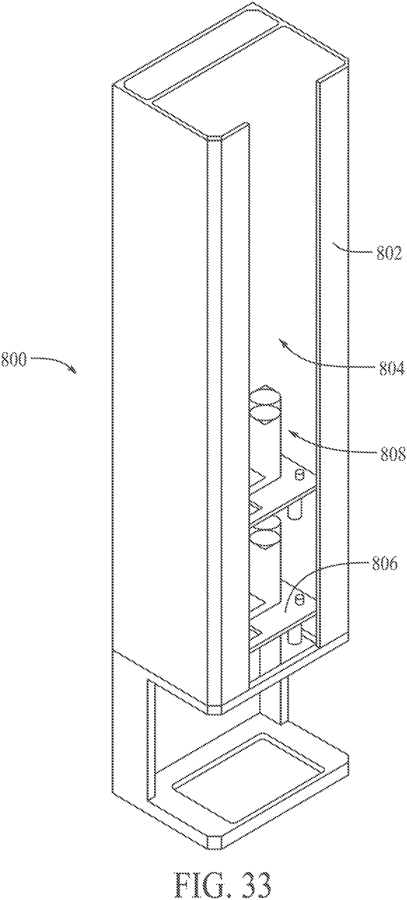

FIG. 33 shows a front perspective view of a cell processing module dispenser 800, which can be a component of the disclosed cell processing system 100. Optionally, the fluid handling dispense 800 can be a component of the fluid handling device 105. Alternatively, the cell processing module dispenser 800 can be a component of the fluid handling device 400 (e.g., a removable component of the fluid handling device 400). The cell processing module dispenser 800 can include a housing 802 that defines a channel 804, and cell processing modules 806, 808. As illustrated, the channel 804 has a smaller width than the width of each of the cell processing modules 806, 808, and the cell processing modules 806, 808 may be slidably inserted and retained within the housing 802 (e.g., without falling through the channel 804). Each of the cell processing modules 806, 808 may be actuated in order to introduce fluid into a flow circuit of the disclosed cell processing system 100.

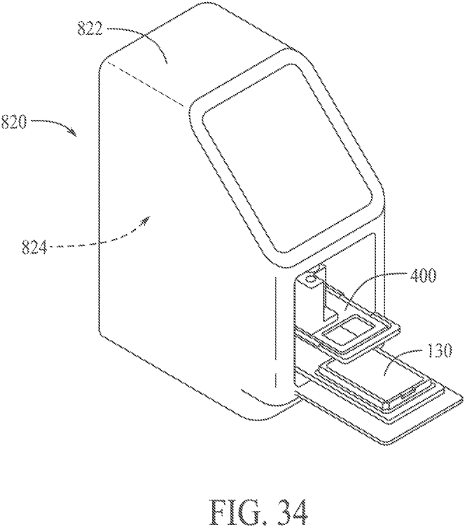

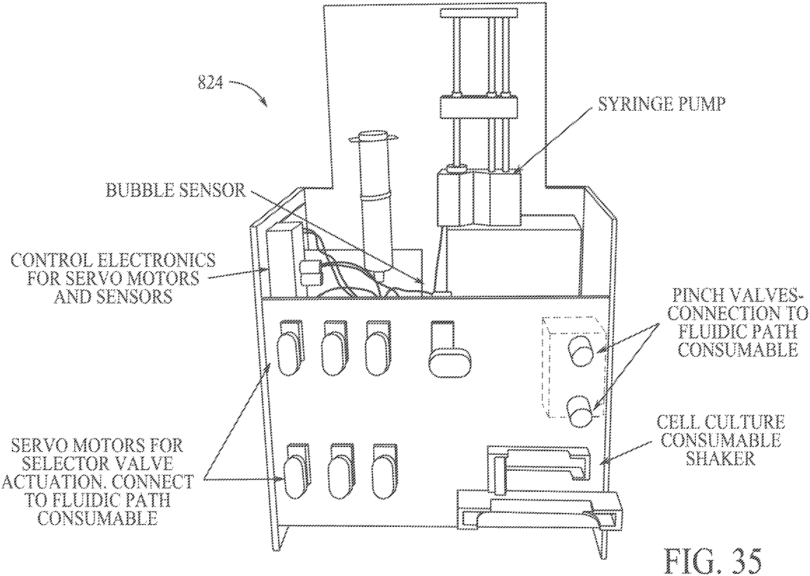

FIG. 34 shows a front perspective view of a cell processing system 820 that is a specific configuration of the cell processing system 100. The cell processing system 820 can include a housing 822, a fluid handling device 400, a cell culture container 130 engaged with the fluid handling device 400, and a fluid handling device 824 contained with the housing (e.g., a specific configuration of the fluid handling device 105) that is in fluid communication with the fluid handling device 400.

FIG. 35 shows a front view of one embodiment of a fluid handling device 824. As shown, the fluid handling device 824 can include a syringe pump to move precise amounts of liquids in the cell processing modules, a bubble sensor to detect the air/liquid interface in the line that connects to the syringe pump so that an accurate determination of the aspiration of variable volumes is achieved, control electronics (e.g., a processor, memory, communization device, etc.) to control the valves and motors, pinch valves to block/open flow through a respective conduit, and servo motors to adjust the position of a rotational valve. In some configurations, the fluid handling device 824 can include a shaker to agitate the cell culture container (e.g., to re-suspend the cells and mix the culture media). The pinch valves and servo motors can adjust how fluid is moved throughout the fluid handling device or the cell processing module and the fluid handling device (e.g., to ensure that only one cell processing module is used at a time to process cells from the cell culture container). In some embodiments, the sterility of the inner lumens of the cell processing modules and cell culture containers is achieved by a gas permeable, sterile barrier such as a 0.22 um PTFE filter.

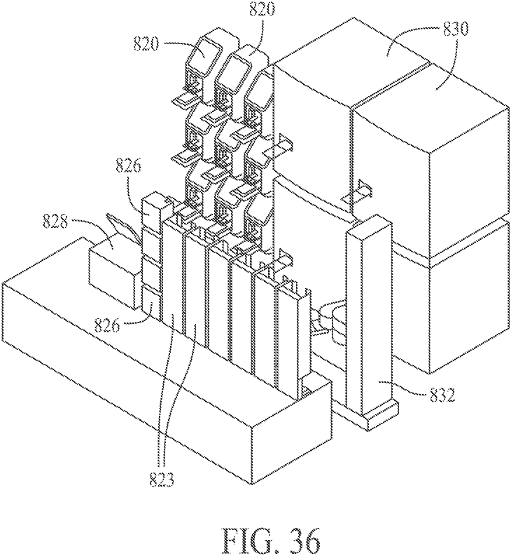

FIG. 36 shows a front perspective view of a plurality of cell processing systems 820 that can operate independently. The cell processing systems may operate in a series in which cells are processed in a first cell processing system 820 and then transferred to a different cell processing system 820. The cell processing systems 820 also may operate in parallel, in which multiple different cell cultures are processed in multiple different cell processing systems. FIG. 36 also shows sampling instruments 826, analytical equipment 828 (e.g., a Flex 2 chemistry, or a cell density and viability analyzer, which is able to analyze the composition of cell media for pH, dissolved oxygen, glucose, lactate, ions (K+, Na+, Ca++) and determine through staining and subsequent imagining if cells are dead or alive), cell processing module dispensers 823 each of which corresponds to a particular cell processing module, automated incubators 830 that can receive one or more of the cell processing systems 820 (or cell culture containers within the cell processing systems) to provide a controlled temperature for cell growth, and a robotic arm 832 for transporting the cell culture containers and the cell processing modules between the automated incubators 830.

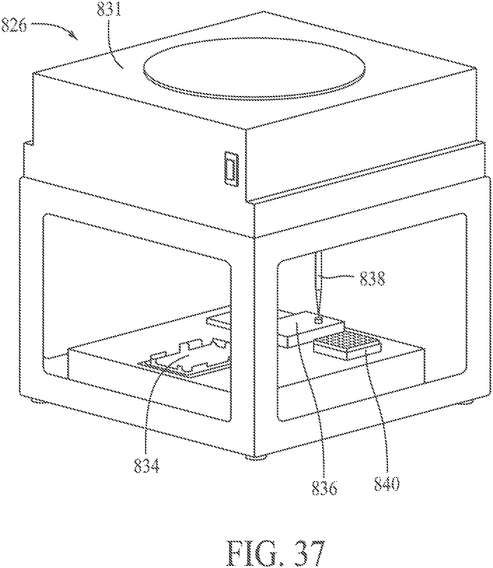

FIG. 37 shows a perspective view of one embodiment of a sampling instrument 826. The sampling instrument 826 can include a filtered enclosure 831 (e.g., using a HEPA filtered) that is sealed on all sides, a shaker 834 that is dimensioned and configured to agitate a cell culture container, a cell culture container 836, an electronic pipetting system 838 having a pipettor that is configured to receive liquid from the cell culture container and dispense it into a plurality of storage vials 840 (or a multi-well container).

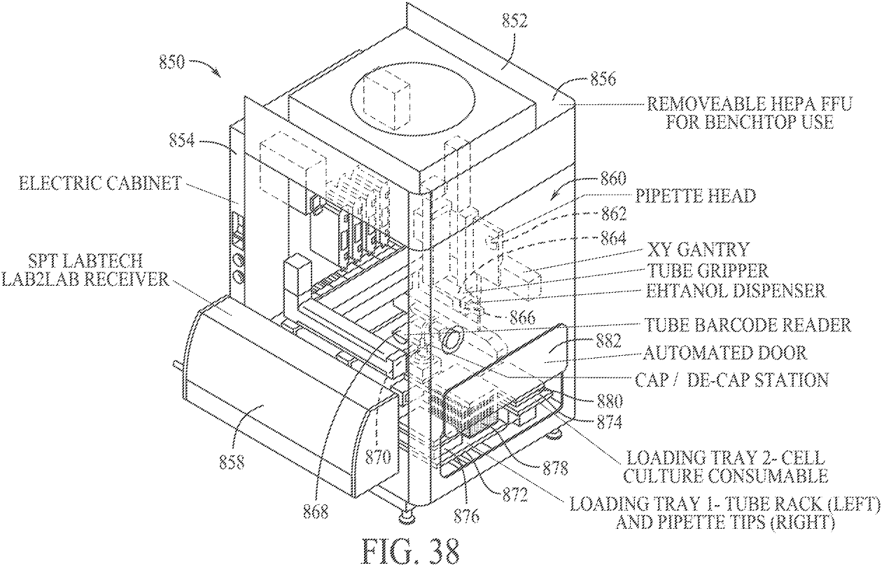

FIG. 38 shows a perspective view of a sampling instrument 850. The sampling instrument 850 can include a housing 852 having an electrical cabinet 854 and a filter 856, a communication system 858, a motion control system 860, a pipette head 862, a gripper 864 (e.g., for tubes, including test tubes), a cleaning liquid dispenser 866 (e.g., for dispensing ethanol), a symbol reader 868 (e.g., for barcodes), and a capping (and uncapping) assembly 870.

The housing 852 can retain and secure some or all of the components of the sampling instrument 850. For example, the electrical cabinet 854 of the housing 852 can retain and house electrical components (e.g., a computing device) used to control one or more aspects of the sampling instrument 850. In some cases, a filter 856, which can be a HEPA filter can be situated on at the top of the housing 852 so as to facilitate filtering of air into and out of the top of the housing 852. In some cases, the sampling instrument 850 can include a trays 872, 874, each of which can be situated within the housing 852. The tray 872 can support a test tube rack 876 having test tubes supported thereon, and can support a pipette tip container 878 having pipette tips supported thereon. The tray 874 can support a cell culture container 880, which can be similar to the other cell culture containers described herein. In some embodiments, the sampling instrument 850 can include a door 882, which can be controlled by a computing device (e.g., that is situated within the housing 852) to selectively allow (and block) access to the trays 872, 874.

The communication system 858 can be in communication with the electrical components of the sampling instrument (e.g., the computing device) and can communicate with other computing devices (e.g., to receive instructions). In some cases, the communication system 858 can be a SPT Labtech Lab2Lab receiver (available from sptlabtech, Melbourn, United Kingdom).

The motion control system 860 can be generally configured to control movement of the pipette head 862 according to a coordinate system. For example, the motion control system 860 can include an X-Y stage (e.g., an x-y gantry) that can control movement of the pipette head 862 along the x and y plane, and include a z-stage that can control movement of the pipette head 862 along the z-axis. In some embodiments, the control system 860 can include a second z-stage that can control movement of the gripper 864, and the cleaning liquid dispenser 866. In some cases, the z-stage that supports the pipette head 862 can also support the gripper 864 and the cleaning liquid dispenser 866.

In operation, a computing device can uncap a test tube from the test tube rack 876 (e.g., using the capping assembly 870), and can cause the gripper 864 to pick up the test tube. Then, a computing device can cause the cleaning liquid dispenser 866 to apply cleaning liquid (e.g., alcohol, including ethanol) to the pipette head 862. After, a computing device can cause the motion control system 860 to move the pipette head 862 to engage a pipette tip (clean) from the pipette tip container 878 thereby securing the pipette tip to the pipette head 862. A computing device can then cause the motion control system 860 to move the pipette head 862 into the interior volume of the cell culture container 880 (e.g., when the septum of the cell culture container has been partially removed), and once within the interior volume of the cell culture container, draw an amount of liquid from the interior volume. After, a computing device can cause the motion control system 860 to move the pipette head 862 to the test tube and dispense the liquid into the test tube. Then, a computing device can cause the motion control system 860 to move the gripper 864 with the test tube to the capping assembly 870 (and symbol reader 868). A computing device can cause the capping assembly 870 to seal the test tube (e.g., by screwing on a cap), and can cause the symbol reader 868 to read the symbol (e.g., barcode) on the test tube for association between the data of the symbol and the contents within the tube.

In some embodiments, the sampling instrument 850 can include a drip catch system. The drip catch system can be designed to contain any unintended drips from the pipette tip while traversing between the cell culture container and tube. The drip catch can include a cup which can contain a greater volume than the complete volume of the pipette tip. This cup can be manufactured from stainless steel or similar cleanable, smooth and corrosion resistant material or coating. This cup can be extended on a mechanism which allows it to fully enclose the pipette tip. The drip catch can provide sufficient clearance below the pipette tip to be deployed while directly above the cell culture container septum and retracted while above the tube (with cap removed). For example, the drip catch (e.g., a cup) can be extended and retracted by an actuator (e.g., a linear actuator) that can be controlled by the sampling instrument. As a more specific example, actuator of the drip catch system can be actuated using compressed air, and can feature a single actuation step for all motion (e.g., the actuator only having two positions - a fully extended position and a fully retracted position).

In some embodiments, the bottom of the cup can have sufficient clearance below the pipette tip so as to not wick droplets from the tip. The drip catch can be mounted to the right side of the pipette head (viewed from the front of instrument). The drip catch system can feature a sterile cleaning solution dispense head (e.g., the cleaning solution being isopropyl alcohol) which can sterilize the cup between uses. In some embodiments, the drip catch having a flat lower surface, and with a smaller volume can be clean (e.g., less surfaces to spray and clean). However, in some cases, the drip catch having a larger volume can decrease the likelihood of blowing contaminates into the space (e.g., interior volume of the housing), but may require more complicated cleaning routines.

In some embodiments, the sampling instrument 850 (or others) can be used to periodically sample the contents within a cell culture container. For example, the tables below show examples of sample processes. In some cases, the CARE Sampler Instrument (e.g., the sampling instrument 850) can be responsible for periodic sampling events occurring within the CARE workflow. These events can occur at least once during all CARE workflow operations after Initial Incubation.

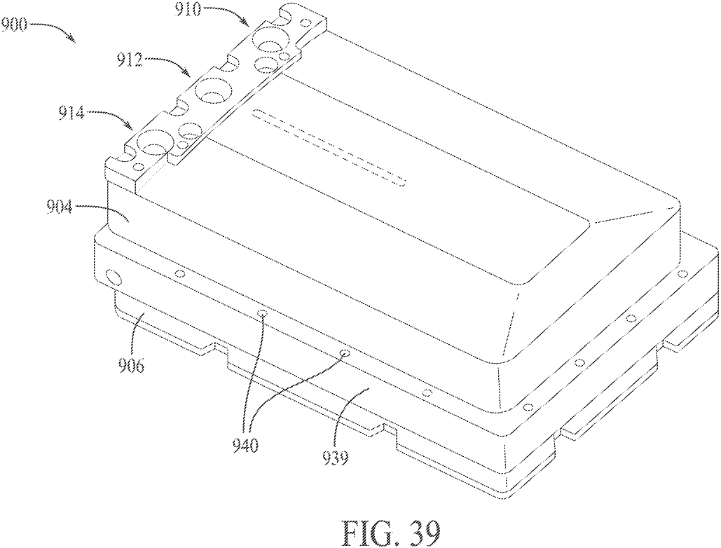

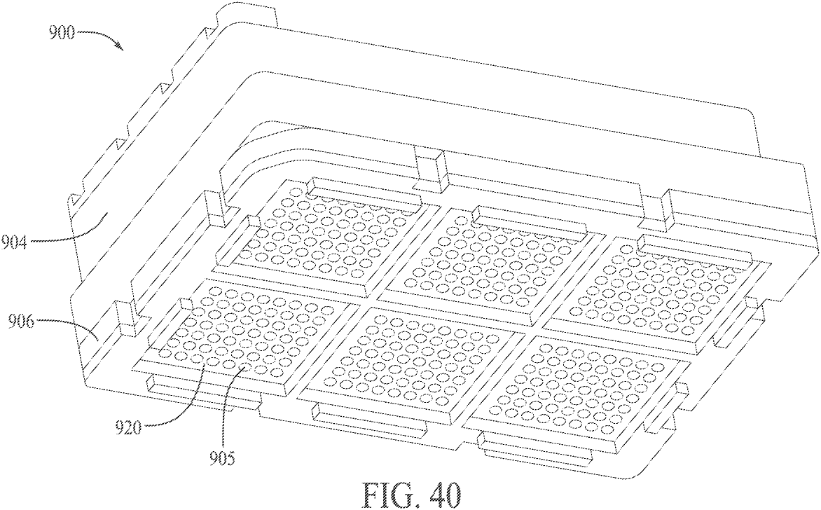

FIGS. 39-42 show another embodiment of a cell culture container 900, which can be similar to the other cell culture containers described herein (e.g., the cell culture containers 130, 160, 200, 250, 300). Thus, the description of the other cell culture containers 130, 160, 200, 250, 300 also pertains to the cell culture container 900. The cell culture container 900 can include a frame 902 having an upper piece 904 and a lower piece 906, a membrane 908, and ports 910, 912, 914. The frame 902 can be coupled to the membrane 908, and can secure the membrane 908 thereto. For example, the membrane 908 can be positioned between the upper piece 904 and the lower piece 906, and a peripheral end of the membrane 908 can be clamped between the pieces 904, 906 (e.g., using one or more threaded fasteners, an adhesive, etc.). In this way, the frame 902 can ensure that a fluid tight seal is created so that liquid contained by the cell culture container 900 is blocked from passing through location in which the pieces 904, 906 clamp the peripheral end of the membrane 908. In some configurations, and as illustrated in FIG. 39, the upper piece 904 of the frame 902 can include a peripheral flange 939 that extends away from a center of the upper piece 904, and the peripheral flange 939 can extend partially (or entirely) around the upper piece 904. The peripheral flange 939 of the upper piece 904 can also include multiple holes 940, each of which can receive a threaded fastener (not shown) for securing the upper piece 904 to the lower piece 906. For example, a peripheral end of the membrane 908 can be positioned between the peripheral flange 939 of the upper piece 904 and the lower piece 906, and the peripheral flange 939 of the upper piece 904 can be coupled to the lower piece 906 (e.g., using one or more threaded fasteners, each of which are received within a respective hole 940 and each of which threadingly engaging the lower piece 906).

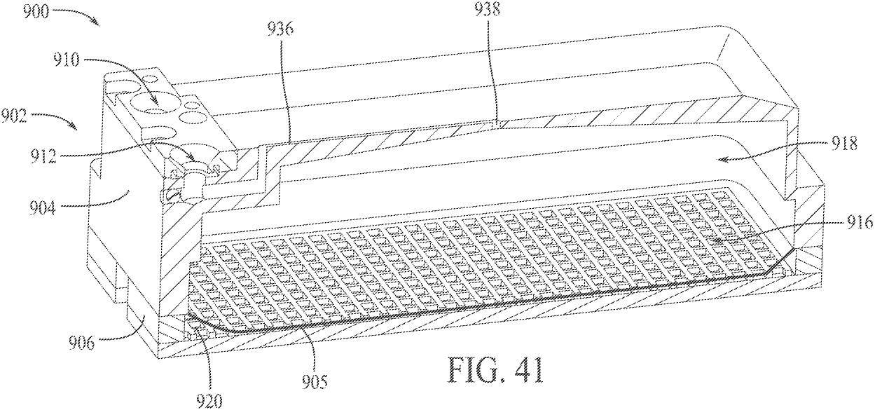

As shown in FIG. 41, the upper piece 904 of the frame 902 and the membrane 908 can define an internal volume 916 of the cell culture container 900 that contains cells and liquid growing media for the cells. For example, the upper piece 904 of the frame 902 can include a cavity 918, and the cavity 918 and the membrane 905 can define the internal volume 916 of the cell culture container 900. In some configurations, the lower piece 906 of the frame 902 can include a substrate 920 that can be gas permeable. For example, the substrate 920 can include a plurality of holes (e.g., positioned in a 2-D array) that facilitate gas diffusion therethrough including oxygen gas, carbon dioxide gas, etc. As a more specific example, the substrate 920 can include a mesh (e.g., a wire mesh, such as, for example, a plastic wire mesh) or other interlaced structure. In some configurations, the substrate 920 can include a region that is substantially (or entirely planar). For example, a central region of the substrate 920 that is positioned central relative to the peripheral end of the membrane 905 can be substantially (or entirely planar). In some configurations, the entire substrate 920 can be substantially (or entirely) planar. Regardless of the configuration, the membrane 905 can contact the region of the substrate 920 that is substantially planar thereby creating a region of the membrane 905 that is substantially planar thereby creating a flat surface for the membrane 905 (e.g., that is positioned more central than the peripheral end of the membrane 905). In this way, the membrane 905, can have a flat surface that provides a consistent distribution of cells for more optimal cell culture conditions. In some embodiments, the membrane 905 is non-expandable. In addition, the membrane 905 can be gas permeable so that the flat surface of the membrane 905 can provide gas exchange with the ambient environment through the substrate 920 (e.g., holes of the substrate 920) to ensure that oxygen gas can enter into the internal volume 916, and that carbon dioxide gas (e.g., as a byproduct of cell growth) exits the internal volume 916 and flows into the ambient environment (e.g., for pH regulation).

In some embodiments, the lower piece 906 of the frame 902 can include legs 922, 924, 926, 928. The legs 922, 924, 926, 928 can each extend away from the lower piece 906 (including the substrate 920) and the upper piece 904 and can contact a supporting surface, such as, for example, a lab bench, a table, etc. In this way, when the cell culture container 900 contacts the support surface (e.g., is supported by the support surface), the substrate 920 is separated from the support surface (e.g., the substrate 920 does not contact the support surface). In some configurations, the legs 922, 924, 926, 928 can define channels 930, 932, 934 that can facilitate gas exchange between the internal volume 916. For example, the legs 922, 924 can define the channel 930, the legs 924, 926 can define the channel 932, and the legs 926, 928 can define the channel 934. Each channel 930, 932, 934 can facilitate gas exchange between the internal volume 916 of the cell culture container 900 and the ambient environment. For example, when the cell culture container 900 is supported by the support surface, the channels 930, 932, 934 provide flow paths for gas exchange between the internal volume of the cell culture container 900 and the ambient environment, via the substrate 920 and the membrane 905. In some cases, without the channels 930, 932, 934, the substrate 920 would undesirably directly contact the support surface, and thus gas exchange between the ambient environment and the internal volume 916 of the cell culture container 900 would be undesirably decreased.

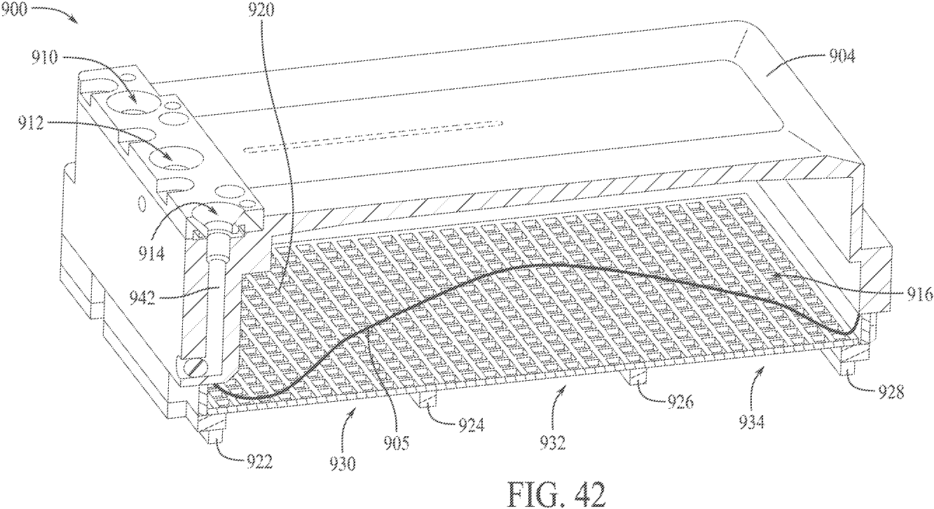

As shown in FIGS. 41 and 42, the upper piece 904 of the frame 902 can include the ports 910, 912, 914, however, in other configurations, the ports 910, 912, 914 can be directed through different components of the cell culture container 900. The upper piece 904 of the frame 902 can also include conduits to facilitate fluid flow through the ports 910, 912, 914 to (and from) the internal volume 916 of the cell culture container 900. For example, the port 912 can be in fluid communication with the internal volume 916 of the cell culture container 900, via a conduit 936 that passes through a wall the upper piece 904 of the frame 902 and terminates at a port 938 in the upper piece 904 of the frame 902. In other words, the conduit 936 provides fluid communication between a top region of the internal volume 916 of the cell culture container 900 and the port 912. Thus, in some cases, the port 912 can facilitate removal or addition of gas into the internal volume 916 at a top region of the internal volume 916 thereby regulating gas located within the internal volume 916 (e.g., by venting gas from the internal volume 916). In this way, the pressure within the internal volume 916 of the cell culture container 900 can be changed, via fluid flow through the port 912. In some embodiments, the port 938 can be positioned at a central region of the cell culture container 900 (e.g., an axis that passes through a centroid of the cell culture container 900 passes through the central region of the cell culture container 900).

In some embodiments, and as illustrated in FIG. 42, the port 914 can be in fluid communication with the internal volume 916 of the cell culture container 900 at a location within the internal volume 916 that is below the port 938 (e.g., so that liquid that passes through the port 914 enters the internal volume 916 of the cell culture container 900 at a lower portion of the cell culture container 900). For example, the port 914 can be in fluid communication with the internal volume 916 of the cell culture container 900, via a conduit 942 that extends downwardly from the port 914 and which is in fluid communication with the internal volume 916 proximal to a lower surface of the upper piece 904 of the frame 902. In this way, the liquid can be directed through the port 914 and into the internal volume 916 of the cell culture container 900 so that liquid enters the internal volume 916 at a lower region rather than an upper region of the internal volume 916 (e.g., so that liquid is more controllably directed into the cell culture container 900, rather than, for example, the liquid spraying from the top of the internal volume 916), which can minimize cell loss during liquid handing procedures. In other words, with liquid (including cells) entering (or exiting) the internal volume 916 of the cell culture container 900 at a lower portion of the internal volume 916 ensures that the cells are continually in contact with the liquid. Otherwise, in some cases, if cells (and liquid) enter through the port 938, cells may undesirably contact the gas (e.g., air) within the internal volume 916 and die.

In some embodiments, the port 914 can be used for cell seeding (e.g., seeding cells into the internal volume 916 of the cell culture container 900), media exchange (e.g., replacing spent cell media that is within the internal volume 916 of the cell culture container 900), cell recovery (e.g., harvesting cells within the internal volume 916 of the cell culture container 900), cell sampling (e.g., retrieving some of the cells within the internal volume 916 of the cell culture container 900), etc. In some embodiments, the port 910 can be configured in a similar manner to the port 914. Thus, correspondingly, the port 910 can be in fluid communication with the internal volume 916 of the cell culture container 900 (e.g., at a lower portion of the internal volume 916), and liquid 910 that passes through the port 910 can enter the internal volume 916 of the cell culture container 900 (e.g., at a lower portion of the internal volume 916 below the port 938).

In some embodiments, the ports 910, 912, 914 can each include one or more selectable valves (e.g., solenoid valves) that selectively allow and block fluid flow through the respective port and to (or out of) the internal volume 916 of the cell culture container 900. In some cases, a computing device can cause the one or more selectable valves to open (or close). In some embodiments, and similarly to the other cell culture containers described herein, the ports 910, 912, 914 can each include a septum (not shown) that is pierceable (or a seal that is selectively sealable), so that an aseptic fluid connection can be established between another component and any of the ports 910, 912, 914. In this way, contamination (including from other microorganisms and viruses) from the ambient environment can be avoided.

In some embodiments, the internal volume 916 of the cell culture container 900 can have different amounts. For example, the internal volume 916 of the cell culture container 900 can be greater than or equal to 200 mL, greater than or equal to 250 mL, etc. In some cases, the internal volume 916 of the cell culture container 900 can be substantially 200 mL or substantially 250 mL, or substantially 500 mL, or substantially 750 mL. In some cases, the internal volume 916 of the cell culture container 900 can be in a range of substantially 200 mL to substantially 750 mL, or substantially 200 mL to substantially 500 mL, or substantially 250 mL to substantially 750 mL, or substantially 250 mL to substantially 500 mL, etc.

In some embodiments, the cell culture container 900 can be used for cell seeding, media exchange, cell recovery, etc. For example, during a cell seeding process, liquid including cells (e.g., suspended therein) pass through the port 914 (e.g., the port 914 being open) and enter the internal volume 916 of the cell culture container 900 at a lower portion of the internal volume 916. Correspondingly, as the liquid passes through the port 914, the port 912 is open so that excess air within the internal volume 916 can pass through the port 912 to be vented to a different component (or to atmosphere). In some cases, the port 912 can be opened (and closed) to ensure that the pressure within the internal volume 916 of the cell culture container 900 is greater than or equal to the atmospheric pressure of the ambient environment (e.g., to ensure that the membrane 905 maintains a substantially planar region). After the liquid enters the internal volume 916 of the cell culture container 900, the ports 912, 914 can close to maintain an aseptic environment within the cell culture container 900.

As another example, during a media exchange process, waste media that includes the cells within the internal volume 916 of the cell culture container 900 is extracted through the port 914. Similarly to the cell seeding process, the port 912 can be open during the media exchange process so that excess air within the internal volume 916 of the cell culture container 900 can be vented to a different component (or to atmosphere). In this way, the port 914 can regulate the air pressure within the internal volume 916 of the cell culture container 900. In some cases, a pressure regulator can be in fluid communication with the port 914, so that when the port 914 is open, the (air) pressure within the internal volume 916 of the cell culture container 900 maintains a consistent pressure. Similarly to the cell seeding process, during the media exchange process, for example, after the liquid has entered the internal volume 916 of the cell culture container 900, the ports 912, 914 can close. As yet another example, during a cell recovery process (e.g., sampling or harvesting), the port 912 can be open so that the liquid within the internal volume 916 of the cell culture container 900 that includes cells is extracted out through the port 912. In some cases, including when a portion of the liquid (e.g., a fourth or a fifth of the liquid) that was originally within the internal volume 916 of the cell culture container 900 remains in the internal volume 916 of the cell culture container 900, the port 914 can be closed (and air can be forcefully drawn out of from the internal volume 916 of the cell culture container 900 through the port 914, via, for example, a pump). In some cases, during the cell recovery process, the pressure within the internal volume 916 of the cell culture container 900 can be less than the atmospheric pressure of the ambient environment (e.g., so that the membrane 905 is forced upwardly due to the atmospheric pressure being greater than the pressure within the cell culture container 900). In this way, the membrane 905 is drawn towards the upper piece 904 (and the port 938) creating a convex region of the membrane 905 (e.g., that is centrally located on the membrane 905). The creation of the convex region that extends towards the upper piece 904 advantageously forces the remaining liquid within the internal volume 916 of the cell culture container 900 to flow down to pass through the port 914. In other words, the deformation of the membrane 905 creates a channel with the membrane 905 to guide the liquid to the port 914.

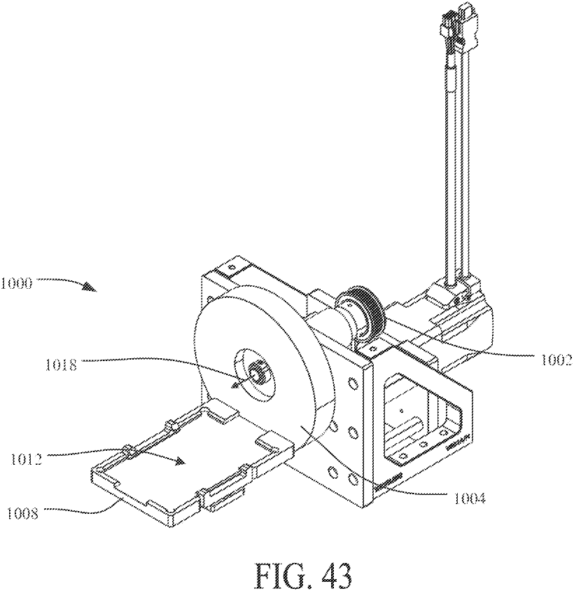

FIG. 43 shows an isometric view of a mixer system 1000, which can be configured to mix a cell culture container (e.g., including any of the cell culture containers described herein). The mixer system 1000 can include a motor 1002, a rotor 1004, and a gripper assembly 1006 including grippers 1008, 1010. Each gripper 1008, 1010 can include a respective recess 1012, 1014, that is configured to receive a cell culture container 1016 (e.g., which can be implemented as any of the cell culture containers described herein). For example, the shape of each recess 1012, 1014 can correspond to the shape of the cell culture container 1016 so that the cell culture container 1016 nests within each recess 1012, 1014. In some specific cases, the cell culture container 1016 can be rectangular, and thus the recesses 1012, 1014 can also be rectangular. Regardless of the configuration, the gripper assembly 1006 can releasably secure the cell culture container 1016 during mixing of liquid within the cell culture container 1016.

In some embodiments, the gripper assembly 1006 can be coupled to the rotor 1004, and in particular, the grippers 1008, 1010 can be coupled to and slidably engaged to the rotor 1004. For example, each gripper 1008, 1010 can be slidably engaged with the rotor 1004 (e.g., each gripper 1012, 1014 sliding along a recess in the rotor 1004) so that the cell culture container 1016 can be placed into engagement with the grippers 1008, 1010 and slid out of engagement with the grippers 1008, 1010. In some cases, the grippers 1008, 1010 can be biased into engagement with the cell culture container 1016 (e.g., to prevent movement between the grippers 1008, 1010 and the cell culture container 1016 during mixing), or the grippers 1008, 1010 can be locked into engagement with each other with the cell culture container 1016 positioned between the grippers 1008, 1010 using, for example, a lock (not shown).

In some embodiments, the motor 1002 can be rotatably coupled to the rotor 1004 (e.g., via gears, a pulley, etc.), so that rotation of the motor 1002 drives rotation of the rotor 1004. For example, the rotor 1004 can rotate about an axis 1018 in a first direction (e.g., a clockwise direction) and the rotor 1004 can rotate about the axis 1018 in a second direction (e.g., a counterclockwise direction) that is opposite to the first direction, each of which can be driven by rotation of the motor 1002. In some cases, the motor 1002 can rotate the rotor 1004 and the cell culture container 1016 engaged with the grippers 1008, 1010 at a rate of substantially 1 Hz, 2 Hz, 3 Hz, 4 Hz, 5 Hz, in the first rotational direction (or the second rotational direction). In some cases, the motor 1002 can switch the rotational direction from the first direction to the second direction (and vice versa), after, for example, the motor 1002 has caused the rotor 1004 to spin in one direction. For example, the motor 1002 can cause the rotor 1004 (and the cell culture container 1016) to rotate in the first direction at a number of Hz (e.g., substantially three Hz), then the motor 1002 can cause the rotor 1004 (and the cell culture container 1016) to rotate in the second direction at a number of Hz. In this way, by switching the rotational direction, the liquid within the cell culture container 1016 is more likely to be mixed more thoroughly (as opposed to only mixing in the same direction) as switching rotational directions can introduce turbulence of the liquid within the cell culture container 1016 that better mixes the liquid.

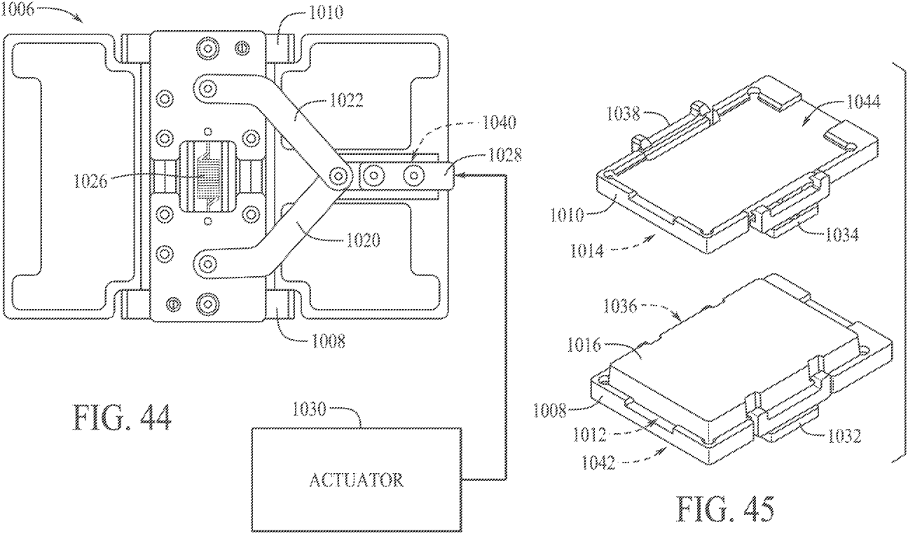

FIG. 44 shows a front view of the gripper assembly 1006 of the mixer system 1000. The gripper assembly 1006 can include the grippers 1008, 1010, arms 1022, 1024, a spring 1026, a slide 1028, and an actuator 1030. The grippers 1008, 1010 can each include the respective recess 1012, 1014, and can include respective engagement features 1032, 1034. The engagement features 1032, 1034 can be implemented in a similar manner as the other engagement features described herein (e.g., the engagement features 236, 472, 474), or the other alignment features described herein (e.g., the alignment features 154, 188). For example, as shown in FIG. 45, the engagement feature 1032 includes a recess, while the engagement feature 1034 includes a protrusion. In this way, when the grippers 1008, 1010 are closed together, with the cell culture container 1016 positioned between the grippers 1008, 1010, the engagement features 1032, 1034 contact each other, with the protrusion of the engagement feature 1034 being inserted into the recess of the engagement feature 1032. In this way, the engagement features 1032, 1034 can help to ensure that the cell culture container 1016 is secured during, for example, the mixing process. In some embodiments, the gripper 1008, 1010 can include additional engagement features. For example, the gripper 1008 can include an engagement feature 1036, while the gripper 1010 can include an engagement feature 1038. The engagement features 1036, 1038 can be implemented in a similar manner as the engagement features 1032, 1034. For example, the engagement feature 1036 can include a recess, while the engagement feature 1038 can include a protrusion. While the engagement features 1032, 1036 have been described as including respective recesses, and the engagement features 1034, 1038 have been described as including respective protrusions, a recess and a protrusion can be exchanged as appropriate. For example, the engagement features 1032, 1036 can each include a protrusion, while the engagement features 1034, 1036 can each include a recess.

As shown in FIG. 44, the arm 1020 can be pivotally coupled to the gripper 1008 (e.g., at one end of the arm 1020), and can be pivotally coupled to the slide 1028 (e.g., at the other end of the arm 1020). Similarly, the arm 1022 can be pivotally coupled to the gripper 1010 (e.g., at one end of the arm 1022), and can be pivotally coupled to the slide 1028 (e.g., at the other end of the arm 1022). In some cases, the arms 1020, 1022 can be pivotally coupled to the slide 1028 at the same location on the slide 1028, which can facilitate more uniform movement of the grippers 1008, 1010. The spring 1026 can be coupled to the grippers 1008, 1010, and in particular, one end of the spring 1026 can be coupled to the gripper 1008, and the other end of the spring 1026 can be coupled to the gripper 1010. The spring 1026 can be configured to bias the grippers 1008, 1010 towards a closed position, in which the grippers 1008, 1010 contact each other. In this way, as the grippers 1008, 1010 are moved away from each other (e.g., to load the cell culture container 1016), the spring 1026 forces the grippers 1008, 1010 closed. In this way, the spring 1026 can help to ensure that the grippers 1008, 1010 clamp onto the cell culture continue 1016 to prevent undesired movement of the cell culture container 1016 during a mixing process.

In some embodiments, the slide 1028 can be positioned within a channel 1040 in the gripper assembly 1006 to ensure that the slide 1028 is constrained to translate within the channel 1040. In this way, translation of the slide 1028 is transformed by the arms 1022, 1024 into movement of the grippers 1008, 1012. For example, the actuator 1030, which can be an electrical actuator (e.g., a linear actuator), a pneumatic actuator, etc., and can be advanced to push the slide 1028 towards the grippers 1008, 1010, thereby rotating the arms 1022, 1024, and correspondingly moving (e.g., translating) the grippers 1008, 1010 away from each other. Then, when the actuator 1030 is retreated, the spring 1026 (having been biased), pulls the grippers 1008, 1010 together thereby rotating the arms 1022, 1024 and forcing the slide 1028 to translate away from the grippers 1008, 1010. In some cases, the grippers 1008, 1010, can be positioned within the same or different channels within the gripper assembly 1006 (e.g., that are aligned with each other) to block rotation of the grippers 1008, 1010, from the rotation of the arms 1022, 1024. Regardless of the configuration, the gripper assembly 1006 can be advantageous in that a single actuator (e.g., the actuator 1030) can drive movement of both grippers 1008, 1012 away from each other. In some cases, although the slide 1028 is illustrated as being a slide block, in other configurations, the slide 1028 can have other shapes, such as for, example, a cylinder.

FIG. 45 shows the grippers 1008, 1010 of the gripper assembly 1006 positioned in the open configuration (e.g., after the actuator 1030 has been extended and the spring 1026 biased). For example, with the grippers 1008, 1010 moved away from each other, the cell culture container 1016 is placed into the recess 1012. Then, the grippers 1008, 1010 can be moved back towards each other (e.g., by retreating the actuator 1030) until the gripper 1010 contacts the gripper 1008, which can include, the engagement features 1034, 1038 of the gripper 1010 contacting the corresponding engagement features 1032, 1036 of the gripper 1008. As the grippers 1008, 1010 are moved towards each other, the cell culture container 1016 is received in the recess 1014. In some embodiments, the grippers 1008, 1010 can each include another respective recess directed into an opposing side of the gripper 1008, 1010 as the compared to the respective recesses 1012, 1014. For example, the recess 1012 can be directed into one side of the gripper 1008, and the gripper 1008 can include a recess 1042 directed into the other opposing side of the gripper 1008. Correspondingly, the recess 1014 can be directed into one side of the gripper 1010, and the gripper 1010 can include a recess 1044 directed into the other opposing side of the gripper 1010. In this way, when the mixer system 1000 includes one or more additional gripper assemblies (e.g., similar to the gripper assembly 1006), the one or more additional gripper assemblies can secure additional cell culture containers (e.g., similar to the cell culture containers 1016). For example, another cell culture container can be positioned within the recess 1042, another cell culture container can be positioned within the recess 1044, and the one or more additional gripper assemblies can be secured around the additional cell culture containers.

In some embodiments, each engagement feature 1032, 1034, 1036, 1038 can include a recess and a protrusion. For example, the engagement feature 1032 can include a protrusion positioned on one side of the engagement feature 1032 and a recess positioned on the opposing side of the engagement feature 1032. In this way, an engagement feature of a gripper of another gripper assembly that includes a protrusion can engage with (e.g., be received within) the recess of the engagement feature 1032 to secure another cell culture container.

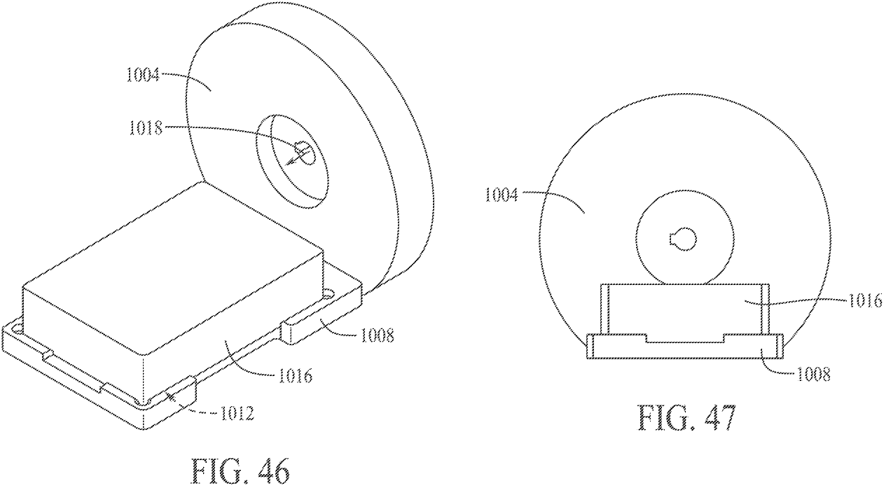

FIG. 46 shows an isometric view of the cell culture container 1016 received within the gripper 1008, and with the gripper 1008 coupled to the rotor 1004, while FIG. 47 shows a top view of the configuration of FIG. 46. In some cases, when the cell culture container 1016 is loaded into the recess 1012 of the gripper 1008, the cell culture container 1016 can be positioned below the axis 1018 of the rotor 1004 (e.g., to ensure that the cell culture container 1016 is stabilized by gravity before the mixing process).

Table 1 below, shows three different mixing schemes: Jurkat 1: Manual Mix (current standard), Jurkat 2: Auto Mix (using the mixer system 1000), and Jurkat 3: Auto Mix (using the mixer system 1000). The following process was used to generate the data in Table 1: 1. Perform mixing routine; 2. Sample 400 uL from CCC (post mixing sample); 3. Remove remaining liquid from CCC into 50 mL falcon tube; 4. Hand mix 50 mL tube; and 5.Sample 400 uL from tube (true cell density/viability sample).

Table 1

Results from the mixing schemes

| | Sample | Cell Density [cells/ml] | | | Viability [%] | | |
|---|---|---|---|---|---|---|---|
| | | Count 1 | Count 2 | Count 3 | Count 1 | Count 2 | Count 3 |
| Post Mixing Sampling | Jurkat #1 | 1.96E+06 | 2.22E+06 | 2.22E+06 | 88.40% | 89.00% | 88.10% |
| | Jurkat #2 | 1.54E+06 | 1.52E+06 | 1.57E+06 | 89.50% | 87.70% | 87.80% |
| | Jurkat #3 | 1.19E+06 | 1.23E+06 | 1.28E+06 | 94.60% | 95.60% | 93.80% |
| True Cell Density and Viability | Jurkat #1 | 2.13E+06 | 2.08E+06 | 2.17E+06 | 78.40% | 76.10% | 76.90% |
| | Jurkat #2 | 1.58E+06 | 1.69E+06 | 1.61E+06 | 81.00% | 78.10% | 79.60% |
| | Jurkat #3 | 1.32E+06 | 1.41E+06 | 1.42E+06 | 90.80% | 91.60% | 91.90% |

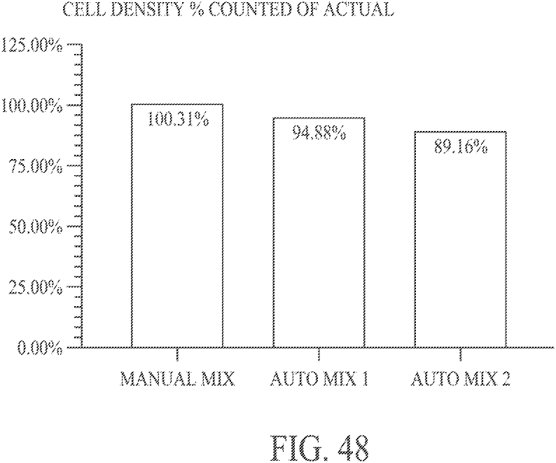

FIG. 48 shows a graph of the cell density divided by the true cell density as a percent for each mixing routine. From these results, the mixer system 1000 is able to homogenize the cells and media within an acceptable range (+/- 10%) of the true cell density of the mixture inside the consumable for sampling requirements.

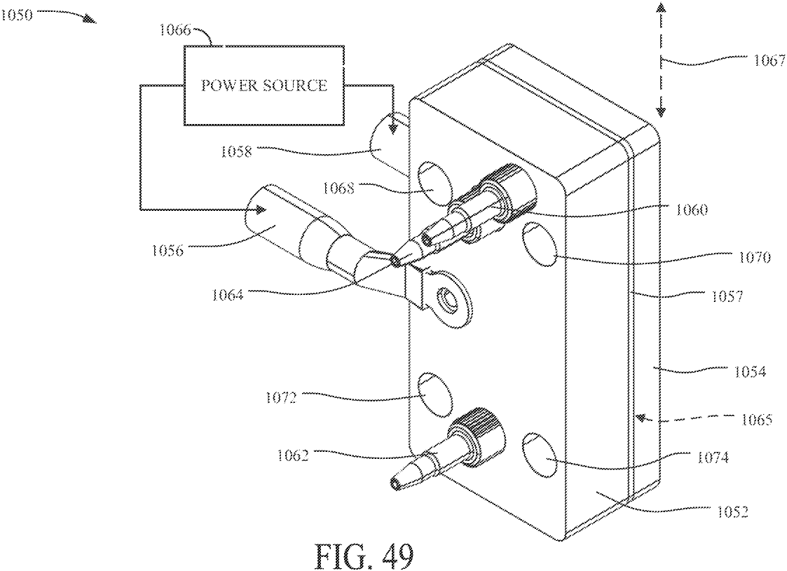

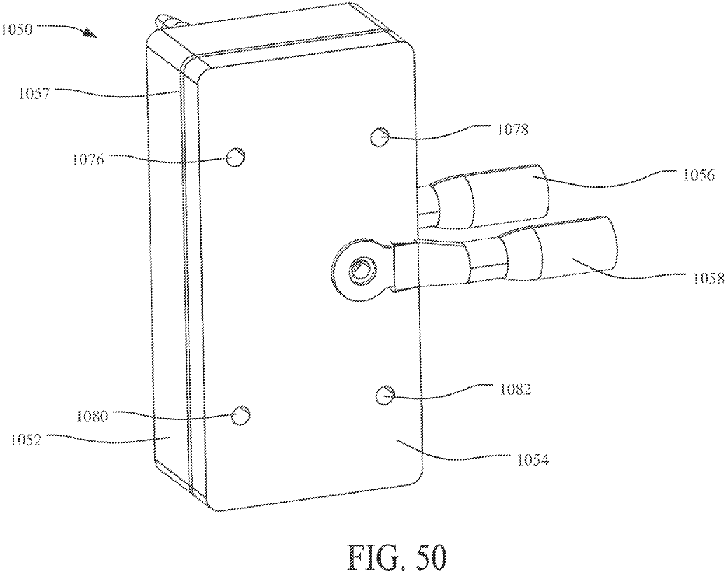

FIG. 49 shows an isometric front view of an electroporator module 1050 that is configured to electroporate cells from a cell culture container (e.g., any of the cell culture containers described herein), and FIG. 50 shows an isometric rear view of the electroporator module 1050. The electroporator module 1050 can be an example of any of the cell processing modules described herein (e.g., the cell processing modules 114, 116, 266, 268, 270, 310, 440, 442, 444, 446, 454, 554). The electroporator module 1050 can include electrodes 1052, 1054, a spacer 1057 positioned between the electrodes 1052, 1054, electrical terminals 1056, 1058, and ports 1060, 1062, 1064. The electrical terminal 1056 can be coupled to the electrode 1052, while the electrical terminal 1058 can be coupled to the electrode 1054. In some embodiments, the electroporator module 1050 can be removably coupled to an electroporator instrument (e.g., of a fluid handling device). For example, electrical terminals of the electroporator instrument can be removably coupled to the electrical terminals 1056, 1058 to electrically connect (and disconnect) the electroporator instrument to the electroporator module 1050. In some cases, the electroporator module 1050 can be a cartridge having a housing (not shown), and the components of the electroporator module 1050 can be positioned within the housing and can be isolated from the ambient environment.

As shown in FIG. 49, the spacer 1057 is positioned between the electrodes 1052, 1054. In addition, the electrodes 1052, 1054 and the spacer 1057 can have the same shape (e.g., the same perimeter shape), and edges of the electrodes 1052, 1054 and edges of the spacer 1057 can be flush. In some configurations, the spacer 1057 can have a thickness that is less than the thickness of the electrodes 1052, 1054. In this way, greater electrical fields can be created at least because surfaces of the electrodes 1052, 1054 can be closer together (e.g., due to the thickness of the spacer 1057 being relatively small). In some cases, the thickness of the electrode 1052 is larger than the thickness of the electrode 1054. However, in alternative configurations, the electrodes 1052, 1054 can have thicknesses that are substantially the same. In some embodiments, the electrodes 1052, 1054 can be coupled together (e.g., using a fastener, such as, for example, a threaded fastener, an adhesive, etc.), with the spacer 1057 positioned between the electrodes 1052, 1054. For example, the electrode 1052 can include holes 1068, 1070, 1072, 1074, and the electrode 1054 can include holes 1076, 1078, 1080, 1082. The electroporator module 1050 can include multiple threaded fasteners (not shown), with each threaded fastener being inserted through a respective hole 1068, 1070, 1072, 1074, and threadingly engaged with a respective hole 1078, 1076, 1082, 1080 (e.g., with the holes 1078, 1076, 1082, 1080 each being threaded), or inserted through a respective hole 1078, 1076, 1082, 1080 and threadingly engaged with a nut (not shown) to couple the electrodes 1052, 1054 together.

The electrical terminals 1056, 1058 can be removably coupled to a power source 1066, which can be part of the electroporator module 1050, or can be part of a fluid handling device (or a receptacle) that has been described previously. The power source 1066 can include an electrical power source (e.g., a battery), and corresponding electrical terminals each of which is removably coupled to a respective electrical terminal 1056, 1058. In this way, the power source 1066 can power the electrodes 1052, 1054 (e.g., with the power source 1066 applying a voltage across the electrodes 1052, 1054) thereby creating an electrical field that electroporates cells. While the electrical terminals 1056, 1058 are illustrated as being ring terminals, in other configurations, the electrical terminals 1056, 1058 can be implemented in different ways, including being, for example, an electrical pin, and electrical socket, etc.

In some embodiments, the ports 1060, 1062, 1064 can each be in fluid communication with each other. For example, the port 1060 can be an inlet, and the port 1062 can be an outlet so that liquid that enters and passes through the port 1060 can flow through the electroporator module 1050 and can pass through the port 1062. The port 1064 can be configured to vent excess gas (e.g., air) when liquid flows from the port 1062 to the port 1064. In this way, air is blocked from being trapped within the electroporator module 1050 during the electroporation process (e.g., when power is provided to the electrodes 1052, 1054), which could otherwise undesirably impact the electroporation process). The electroporator module 1050 can include a channel 1065 that is positioned between the electrodes 1052, 1054, and is in fluid communication with the ports 1060, 1062, and the port 1064 (e.g., that vents excess air). A longitudinal dimension of the channel 1065 can extend along a longitudinal axis 1067 that is parallel to a longitudinal dimension of the electrodes 1052, 1054. In this way, the longitudinal dimension of the channel 1065 is substantially perpendicular to the electric field generated between the electrodes 1052, 1054. Correspondingly, the electric field that is generated between the electrodes 1052, 1054 is substantially perpendicular to a flow path from the port 1060, through the channel 1065, and out through the port 1062.

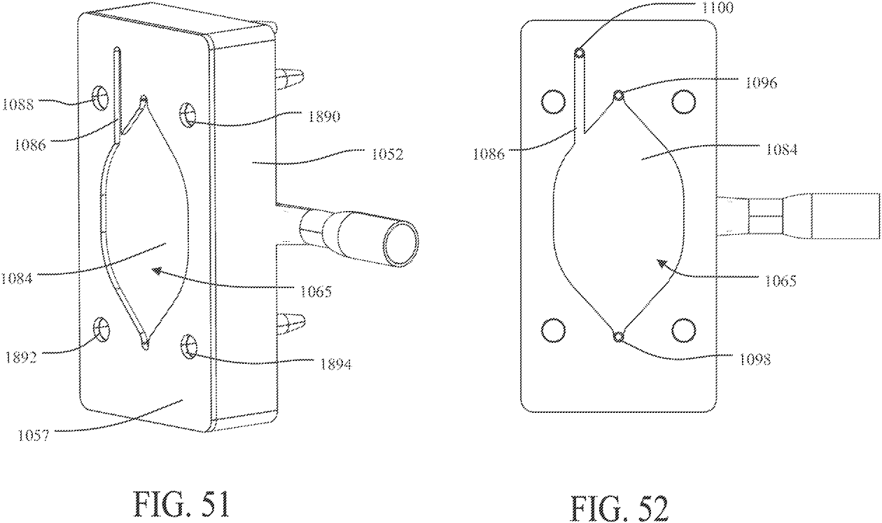

FIG. 51 shows a front isometric view of the electrode 1052 and the spacer 1057, with the electrode 1054 removed for visual clarity. As shown in FIG. 51, the spacer 1057 can include a cutout 1084 and a channel 1086. In some cases, the cutout 1084 can define the channel 1065, and thus the cutout 1084 can be in fluid communication with the ports 1060, 1062. Correspondingly, the cutout 1084 can be in fluid communication with the channel 1086, with the channel extending away from the cutout 1084, and thus the cutout 1084 can be in fluid communication with the port 1064 (e.g., so that air within the cutout 1066 can be vented out through the port 1064). While the cutout 1084 (and a portion of the channel 1065) is illustrated in FIG. 51 as being eye-shaped, in other configurations, the cutout 1084 can have other shapes, such as, for example, being ovoid, linear, etc. In some cases, the spacer 1057 can also include circular cutouts 1088, 1890, 1892, 1894, each of which is configured to align with a respective hole 1070, 1068, 1074, 1072 of the electrode 1052, and a respective hole 1076, 1078, 1080, 1082 of the electrode 1054.

FIG. 52 shows a side view of the electrode 1052 and the spacer 1057 of FIG. 51. As shown in FIG. 52, the electrode 1052 can include channels 1096, 1098, 1100 directed through the electrode 1052. The channels 1096, 1098, 1100 can each be in fluid communication with the respective port 1060, 1062, 1064. Thus, the channels 1096, 1098, 1100 can facilitate fluid flow through the electroporator module 1050. For example, liquid can flow through the port 1060, through the channel 1096, through the cutout 1084, through the channel 1098, and out through the port 1062. Correspondingly, including as liquid flows through the cutout 1084, gas can flow through the channel 1086, through the channel 1100, and out through the port 1064.

In some embodiments, the spacer 1057 can be formed out of an insulating material, which can be different than the materials of the electrodes 1052, 1054. In this way, the spacer 1057 does not undesirably interact with the electric field produced by the electrodes 1052, 1054. In some cases, the spacer 1057 defining fluid flow through the electroporator module 1050 can be desirable rather than, for example, the electrodes 1052, 1054 (e.g., one of the electrodes 1052, 1054 including channels directed therein) at least because the respective surfaces of the electrodes 1052, 1054 can remain planar (e.g., not including the cutout 1066), which can provide a more uniform electric field along the length of the cutout 1084.

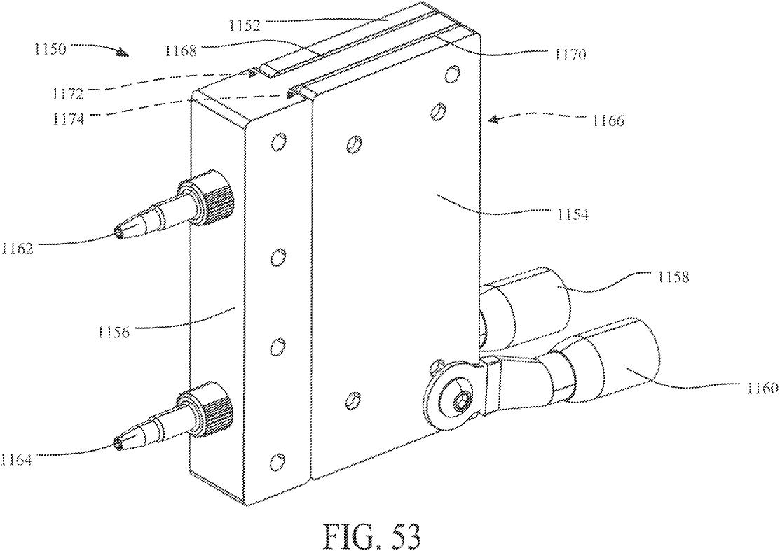

FIG. 53 shows an isometric view of an electroporator module 1150, which can be similar to the electroporator module 1050 described above. Thus, the description of the electroporator module 1050 pertains to the description of the electroporator module 1150 (and vice versa). Similarly to the electroporator module 1050, the electroporator module 1150 can also include electrodes 1152, 1154, a spacer 1156, electrical terminals 1158, 1160, ports 1162, 1164 (e.g., with the port 1162 being an inlet, and the port 1164 being an outlet), and a channel 1166 that is in fluid communication with the ports 1162, 1164 and that passes through the electroporator module 1150. In addition, the electroporator module 1150 can include gaskets 1168, 1170, each of which can be substantially planar.

As shown in FIG. 53, the spacer 1156 can include recesses 1172, 1174, each of which is directed into an opposing side of the spacer 1156. The gasket 1168 can be positioned in the recess 1172 and can be in contact with one side of the spacer 1156. Correspondingly, the gasket 1170 can be positioned in the recess 1174 and can be in contact with the other side of the spacer 1156. The electrode 1152 can also be positioned within the recess 1172 and the electrode 1154 and can be in contact with the gasket 1168. Correspondingly, the electrode 1154 can also be positioned within the recess 1174 and can be in contact with the gasket 1170. In some configurations, the electrodes 1152, 1154 can be coupled to the spacer 1156, such as, for example, using one or more threaded fasteners, with the spacer 1156 and the gaskets 1168, 1170 being positioned between the electrodes 1152, 1154.

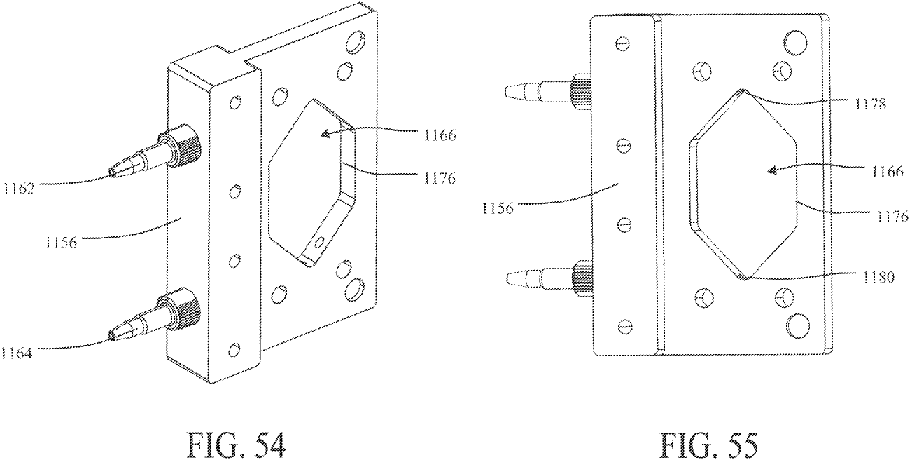

FIG. 54 shows an isometric view of the spacer 1156, while FIG. 55 shows a front view of the spacer 1156. In some embodiments, the spacer 1156 can include the ports 1162, 1164, and can define the channel 1166. For example, the spacer 1156 can include a cutout 1176, and channels 1178, 1180, which can collectively define the channel 1166. Thus, the channel 1178 can be in fluid communication with the port 1162, the channel 1180 can be in fluid communication with the port 1164, and the cutout 1176 can be in fluid communication with the channels 1178, 1180. Accordingly, the liquid can pass through the port 1162, flow through the channel 1178, flow through the cutout 1176, flow through the channel 1180, and flow out through the port 1164. In some cases, a first portion of the width of the cutout 1176 can increase in a direction away from the port 1162, and a second portion of the cutout 1176 different from the first portion can decrease in a direction towards the port 1164.

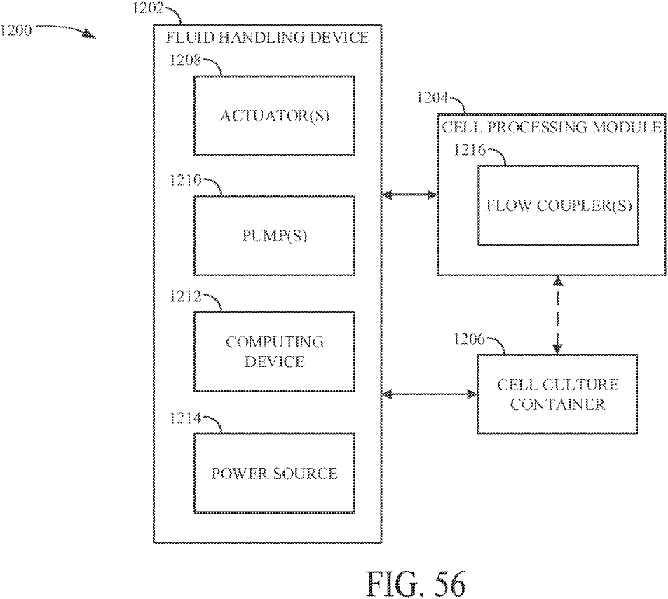

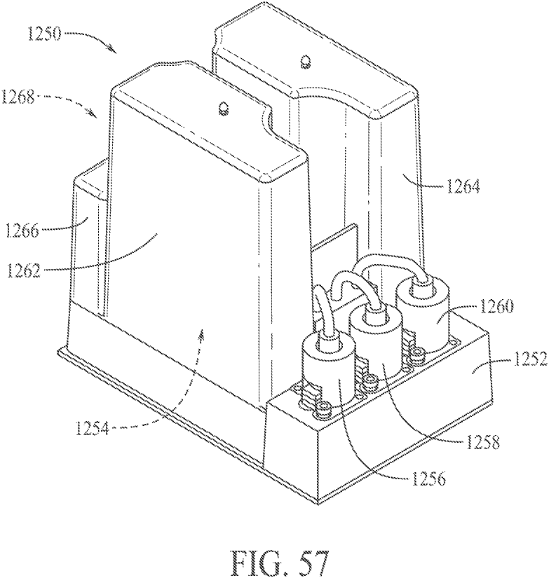

FIG. 56 shows a schematic illustration of a cell processing system 1200, which can be a specific implementation of the cell processing system 100. Thus, the cell processing system 100 pertains to the cell processing system 1200 (and vice versa). Similarly to the cell processing system 100, the cell processing system 1200 can also include a fluid handling device 1202, a cell processing module 1204, and a cell culture container 1206. As shown in FIG. 56, the solid lines indicate mechanical connections between respective components, while the dotted lines indicate fluid communication connections (e.g., pathways) between respective components. For example, the fluid handling device 1202 can be selectively mechanically coupled to the cell processing module 1204, and to the cell culture container 1206. As a more specific example, the cell processing module 1204, which can be a cartridge having a housing, can be received within a recess of the fluid handling device 1202. Correspondingly, the cell culture container 1206, which can also be a cartridge having a housing, can be received within a recess of the fluid handling device 1202 (e.g., a different recess than the recess that receives the cell processing module 1204). In some embodiments, the cell culture container 1206 can be in selective communication with the cell processing module 1204 (e.g., the cell culture container 1206 can be brought into (and out of) fluid communication with the cell processing module 1204), and a flow path within the cell processing module 1204 can be isolated from the ambient environment surrounding the cell processing system 1200 (e.g., during movement of liquid to or from the cell culture container 1206).

Similarly to the other fluid handling devices described herein (e.g., the fluid handling device 105), the fluid handling device 1202 can include actuator(s) 1208, pump(s) 1210, a computing device 1212, and a power source 1214. In some cases, the actuator(s) 1208 can include one or more linear actuators (e.g., electrical linear actuators, pneumatic linear actuators, hydraulic linear actuators, etc.), one or more rotational actuators (e.g., motors that drive rotation of a component, such as, for example, a valve, a pump, etc.). The pump(s) 1210 can be implemented in a similar manner as the other pumps described herein (e.g., the pump(s) 120, 256, 258, 306, 572, 570), the computing device 1212 can be implemented in a similar manner as the other computing devices described herein (e.g., the computing devices 122, 568), and the power source 1214 can be implemented in a similar manner as the other power sources described herein (e.g., the power source 1066). In some embodiments, while the pump(s) 1210 are illustrated as being within the fluid handling device 1202, in other configurations, the cell processing module 1204 can include the pump(s) 1210. In this case, for example, the pump(s) 1210 can engage with respective actuator(s) 1208 (e.g., rotational actuators, such as motors) so that the respective actuator(s) 1208 power the pump(s) 1210 (e.g., with the pump(s) 1210 being positioned within the cell processing module 1204 and isolated from the ambient environment).

In some embodiments, and as described above, the cell processing module 1204 can include flow coupler(s) 1216 that can selectively bring the internal volume of the cell culture container 1206 into (and out of) fluid communication with a flow path within the cell processing module 1204. For example, an actuator 1208 (e.g., a linear actuator) can be aligned with the flow coupler 1216 when, for example, the cell processing module 1204 is engaged with the fluid handling device 105 (e.g., the cell processing module 1204 is received within a recess of the fluid handling device 1202). Then, the actuator 1208 can be extended (e.g., by the computing device 1212) to drive the flow coupler 1216 until the flow coupler 1216 brings the internal volume of the cell culture container 1206 into fluid communication with a flow path of the cell processing module 1204 that is isolated from the ambient environment (e.g., continuously isolated from the ambient environment). Accordingly, liquid from the internal volume of the cell culture container 1206 that can include cells can be processed by the cell processing module 1204. After the cells are processed, the actuator 1208 can be retracted (e.g., by the computing device 1212), and the flow coupler 1216 can disengage the cell culture container 1206 thereby isolating the internal volume of the cell culture container 1206 from the ambient environment. In this way, the cells that grow within the cell culture container 1206 can be processed without undesirably exposing them to the ambient environment, which can undesirably decrease the viability of the cells. In addition, and advantageously, the fluid handling device 1202 is isolated from being in fluid communication (e.g., liquid communication) with the cell processing module 1204, and the cell culture container 1206. In other words, liquid from the cell culture container 1206 (or the cell processing module 1204) does not flow through the fluid handling device 1202. In this way, the fluid handling device 1202 can control routing of fluid from the cell culture container 1206 and to the cell processing module 1204 (and vice versa), without the liquid and the cells therein being contaminated by the fluid handling device 1202.

In some embodiments, the fluid handling device 1202 can include one or more electrical terminals that can engage with one or more electrical terminals of the cell processing module 1204 to selectively electrically connect (and disconnect) the fluid handling device 1202 to the cell processing module 1204. In this way, the cell processing module 1204 can leverage the electrical power from the fluid handling device 1202 (e.g., the power source 1214), and thus the cell processing module 1204 does not need to include a power source that will likely be disposed of after the cell processing step has been completed, which can make the cell processing module 1204 more cost-effective.

In some embodiments, the cell processing module 1204 can include a plurality of tanks, and a plurality of selectable valves that can adjust the fluid communication between the tanks. In some cases, respective actuators 1208 can engage with respective selectable valves to adjust the positions of the selectable valves, via, for example, the computing device 1212.

FIG. 57 shows an isometric view of a cell processing module 1250, which can be a specific implementation of any of the cell processing modules described herein. Thus, the cell processing modules described herein are applicable to the cell processing module 1250 (and vice versa). The cell processing module 1250 can include a housing 1252 that defines an internal volume 1254 isolated from the ambient environment, flow couplers 1256, 1258, 1260, tanks 1262, 1264, 1266, 1268, multi-position valves 1270, 1272, an electrical terminal 1274, and a port 1276. Each of the components of the cell processing module 1250 can be coupled to the housing 1252. For example, the flow couplers 1256, 1258, 1260, the tanks 1262, 1264, 1266, 1268, the multi-position valves 1270, 1272, the electrical terminal 1274, and the port 1276 can be coupled to the housing 1252.

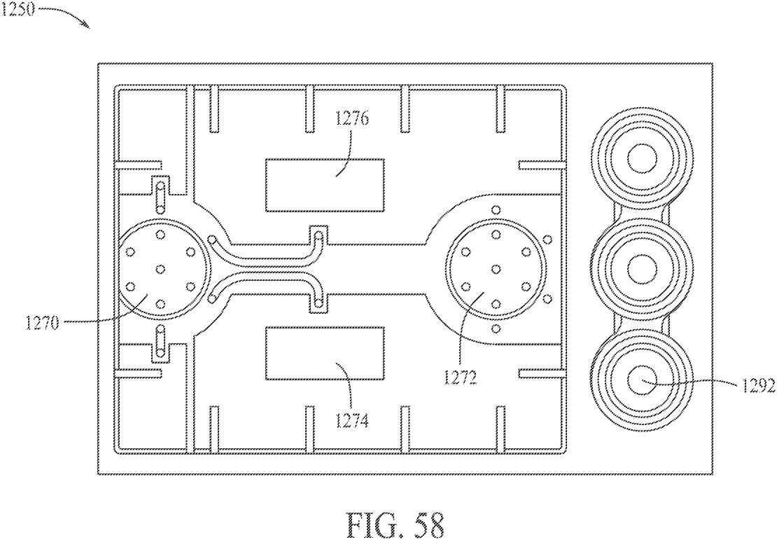

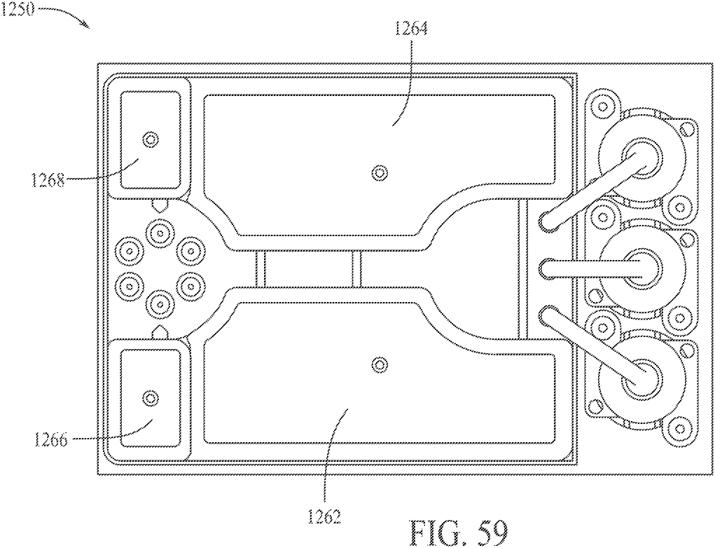

FIG. 58 shows a bottom view of the cell processing module 1250, while FIG. 59 shows a top view of the cell processing module 1250. As shown in FIG. 58, the multi-position valves 1270, 1272, the electrical terminal 1274, and the port 1276 are each positioned on a lower surface of the housing 1252. The multi-position valves 1270, 1272 can include multiple positions and are each configured to bring different components of the cell processing module 1250 into (or out of) fluid communication. For example, the multi-position valve 1270 can be in fluid communication with the multi-position valve 1272, and can be moved to a first position to bring the multi-position valve 1272 into fluid communication with the tank 1262, moved to a second position to bring the multi-position valve 1272 into fluid communication with the tank 1264, moved to a third position to bring the multi-position valve 1272 into fluid communication with the tank 1266, and moved to a fourth position to bring the multi-position valve 1272 into fluid communication with the tank 1268. Correspondingly, the multi-position valve 1272 can be moved to a first position to bring the multi-position valve 1270 into fluid communication with a conduit of the flow coupler 1256, moved to a second position to bring the multi-position valve 1270 into fluid communication with a conduit of the flow coupler 1258, and moved to a third position to bring the multi-position valve 1270 into fluid communication with a conduit of the flow coupler 1260. In some cases, and as described above, a first actuator of a fluid handling device (e.g., the fluid handling device 1202) can engage with and can move the multi-position valve 1270, and a second actuator of the fluid handling device can engage with and can move the multi-position valve 1272.

In some embodiments, one or more electrical components of the cell processing module 1250 (e.g., an electrode) can be electrically connected to the fluid handling device, via connection between the electrical terminal 1274 and a corresponding electrical terminal of the fluid handling device. For example, when the housing 1252 of the cell processing module 1250 is engaged with the fluid handling device, the electrical terminal 1274 connects to a corresponding electrical terminal of the fluid handling device. In this way, electrical power can be provided, from the fluid handling device and to the electrical components of the cell processing module 1250, via the connected electrical terminals. In some cases, in a similar manner as the electrical terminals, the port 1276 can be brought into (and out of) fluid communication with a fluid source of the fluid handling device. For example, when the housing 1252 of the cell processing module 1250 is engaged with the fluid handling device, the port 1276 engages with a corresponding port of the fluid handling device. In this way, fluid from the fluid source (e.g., a pump of the fluid handling device) can be directed through the port of the fluid handling device, and through the port 1276 of the cell processing module 1250. In some cases, the port 1276 can be in fluid communication with the tanks 1262, 1264, 1266, 1268. In this way, fluid (e.g., gas, such as air) can pass into the port 1276 to drive liquid from one of the tanks 1262, 1264, 1266, 1268 to the cell culture container (not shown) via one of the flow couplers 1256, 1258, 1260. Correspondingly, fluid can pass out of the port 1276 to draw liquid out of the cell culture container into one of the tanks 1262, 1264, 1266, 1268, via one of the flow couplers 1256, 1258, 1260.

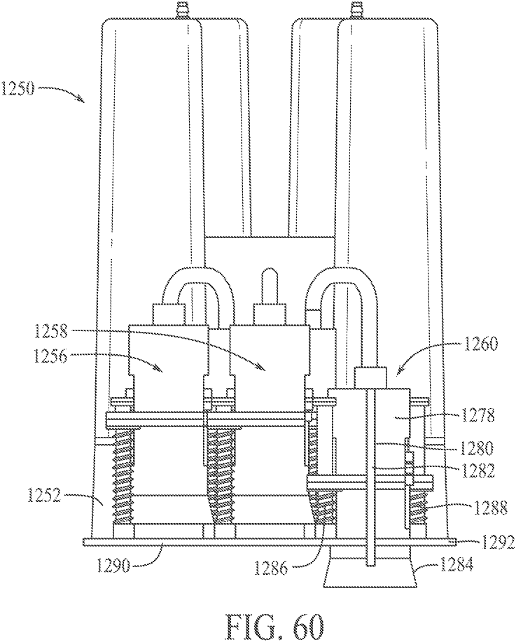

FIG. 60 shows a front view of the cell processing module 1250, with the flow coupler 1260 in an extended position. Each of the flow couplers 1256, 1258, 1260 can be implemented in a similar manner, and so, for the sake of brevity only the flow coupler 1260 will be described. The flow coupler 1260 can include a reciprocating member 1278 (e.g., similar to the reciprocating member 288) that can be a plunger, a hollow tube 1280 coupled to the reciprocating member 1278 defining a conduit 1282, an enclosure 1284 coupled to the reciprocating member 1278, and springs 1286, 1288. As shown in FIG. 60, the enclosure 1284 can partially (or entirely) surround the distal end of the hollow tube 1280 (e.g., which can be a needle), so that, for example, the hollow tube 1280 does not extend past the enclosure 1284. In this way, the enclosure 1284 can act as a shield to block contaminants from being introduced into the cell processing module 1250 (or the cell culture container).

In some embodiments, the springs 1286, 1288 can each be coupled between the reciprocating member 1278 and the housing 1252, and the hollow tube 1280 (and the conduit 1282) can be positioned between the springs 1286, 1288. In some configurations, although two springs 1286, 1288 are shown, in some cases, each flow coupler can include a spring. For example, the spring can be positioned so that the reciprocating member is coaxially positioned within the spring, with the spring coupled between the reciprocating member 1278 and the housing 1252. Regardless of the configuration, as the reciprocating member is depressed, such as, for example, by an actuator (e.g., a linear actuator) of the fluid handling device, the reciprocating member 1278 and the hollow tube 1280 extends until the distal end of the hollow tube 1280 (and an end of the enclosure 1284) extends past a lower surface 1290 of the housing 1252. At this point, the hollow tube 1280 pierces a septum of a cell culture container (not shown) to bring the conduit 1282 in fluid communication with the internal volume of the cell culture container. In some cases, after the cells have been processed, and the processed cells have been inserted back into the internal volume of the cell culture container, the actuator of the fluid handling device can be retracted, and the springs 1286, 1288 that were biased (e.g., compressed) when the reciprocating member 1278 was advanced, retract and force the reciprocating member 1278 upwards to return to the unbiased position.

In some cases, to ensure that the hollow tube 1280 remains free of contamination prior to being directed into the cell culture container, the flow coupler 1260 can include a septum 1292 that can extend across a hole in the housing 1252. In this way, including in configurations in which the enclosure 1284 is removed, the housing 1252 and the septum 1292 can define a cavity that is isolated from the ambient environment. Thus, when cells are to be processed using the cell processing module 1250, the hollow tube 1280 can be extended to pierce through the septum 1292, and subsequently to pierce through the septum of the cell processing module. In other configurations, including prior for engagement of the cell processing module 1250 to a cell culture container, a disinfectant (e.g., isopropyl alcohol, including substantially 70% isopropyl alcohol) can be applied (e.g., swabbed) to the flow coupler (e.g., the flow coupler 1260) to disinfect the flow coupler.

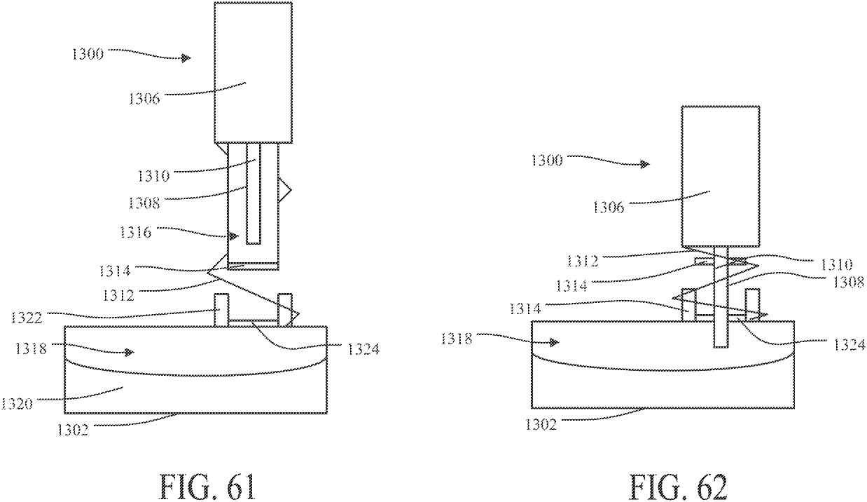

FIG. 61 shows a schematic illustration of a flow coupler 1300 prior to engagement with a cell culture container 1302. The flow coupler 1300 can be implemented in a similar manner as any of the other flow couplers described herein (and vice versa), and the cell culture container 1302 can be implemented in a similar manner as any of the other flow couplers described herein (and vice versa). The flow coupler 1300 can include a reciprocating member 1306 (e.g., a plunger), a hollow tube 1308 coupled to the reciprocating member 1306 defining a conduit 1310 therein, a return spring 1312 coupled to the reciprocating member 1306 (and coupled between the reciprocating member 1306 and a housing of the cell processing module (not shown)), and a septum 1314. In some configurations, the septum 1314 can be coupled to and can extend across a hole of a housing of a cell processing module that includes the flow coupler 1300. In addition, the septum 1314 and the housing of the cell processing module can define a cavity 1316 that is isolated from the ambient environment. In this way, the hollow tube 1308 (e.g., the distal end of the hollow tube 1308) can be positioned within the cavity 1316, which can prevent contamination of the hollow tube 1308 from the ambient environment.

The cell culture container 1302 can define an internal volume 1318, which can include liquid and cells 1320 positioned therein, and can include an extension 1322, and a septum 1324 positioned within a cavity of the extension 1322. As shown in FIG. 61, the internal volume 1318 of the cell culture container 1302 including the liquid and cells 1320 is isolated from the ambient environment.

FIG. 62 shows a schematic illustration of the flow coupler 1300 engaged with the cell culture container 1302. As shown in FIG. 62, the reciprocating member 1306 has been advanced (e.g., by an actuator of the fluid handling device) until the hollow tube 1308 pierces and extends through the septum 1314, the hollow tube 1308 pierces and extends through the septum 1324 into the internal volume 1318 of the cell culture container 1302. In this way, the conduit 1310 is brought into fluid communication with the internal volume of the cell culture container 1302 without the internal volume of the cell culture container 1302 (e.g.., the cells therein) being exposed to the ambient environment. In some embodiments, as the reciprocating member 1306 is advanced, the return spring 1312 loads. In this way, when the actuator that contacts the reciprocating member 1306 retracts, the return spring 1312 unloads to cause the reciprocating member 1306 to retract, thereby retracting the hollow tube 1308 out of the internal volume 1318 of the cell culture container 1302 back through the septum 1324 (e.g., and in some cases back through the septum 1324). In some configurations, each of the septums 1314, 1324 can advantageously retract to reseal. In other words, the septum 1324 retracts around a hole that was created in the septum 1324 from the hollow tube 1308 piercing the septum 1324, so that opposing surfaces that defined the hole contact each other. Stated yet another way, the septum 1324 retracts to reseal the hole that was created in the septum 1324 from the piercing of the septum 1324. In some cases, the septum 1314 can be configured to reseal in a similar manner as the septum 1324. Regardless, by the septum 1314 resealing can advantageously isolate the internal volume 1318 of the cell culture container 1302 from the ambient environment even after the hollow tube 1308 is removed from the cell culture container 1302.

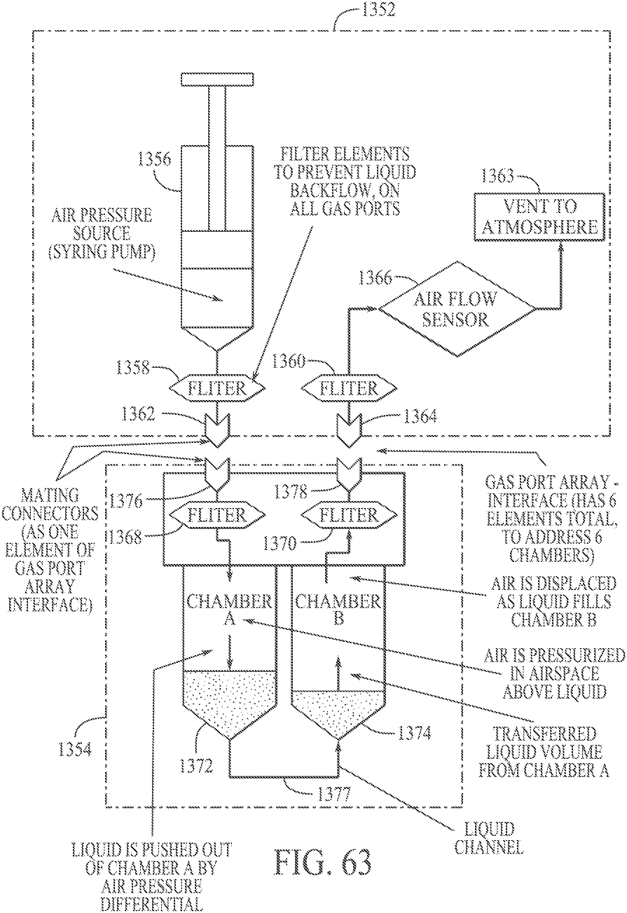

FIG. 63 shows a schematic illustration of a fluid handling device 1352 prior to engagement with a cell processing module 1354. The description of the fluid handling device 1352 is applicable to other fluid handling devices described herein (and vice versa), and the description of the cell processing module 1354 is applicable to other cell processing modules described herein (and vice versa). The fluid handling device 1352 can include a pressure source 1356 (e.g., a pump, such as, a syringe pump), filters 1358, 1360, connectors 1362, 1364, a gas flow sensor 1366 (e.g., an air flow sensor), and a vent 1363 in fluid communication with the atmosphere. The cell processing module 1354 can include filters 1368, 1370, chambers 1372, 1374, a channel 1377, and connectors 1376, 1378.

As shown in FIG. 63, the filter 1358 can be positioned between the connector 1362 and the pressure source 1356, while the filter 1360 can be positioned between the connector 1364 and the vent 1363. Correspondingly, the filter 1368 can be positioned between the chamber 1372 and the connector 1376, while the filter 1370 can be positioned between the chamber 1374 and the connector 1378. The connectors 1362, 1364 of the fluid handling device 1352 are configured to engage the respective connectors 1376, 1378 of the cell processing system 1354 to bring the cell processing module 1354 into fluid communication with the fluid handling device 1352. For example, the connector 1362 can engage with the connector 1376 to bring the pressure source 1356 into fluid communication with the chamber 1372, and the connector 1364 can engage with the connector 1378 to bring the chamber 1374 into fluid communication with the vent 1363. In some embodiments, the channel 1377 can be in fluid communication with the chambers 1372, 1374.

As shown in FIG. 63, when, for example, the chamber 1372 includes liquid positioned therein, the pressure source 1356 can drive first gas through the filter 1358, through the connectors 1362, 1376, through the filter 1368, and into the chamber 1372. At this point, when the first gas is driven into the chamber 1372, the liquid within the chamber 1372 is forced out of the chamber 1372, through the channel 1377, and into the chamber 1374. The liquid that enters the chamber 1374 displaces second gas that is positioned within the chamber 1374, forcing the second gas to flow out of the chamber 1374, through the filter 1370, through the connectors 1378, 1364, through the filter 1360, past the gas flow sensor 1366, and out through the vent 1363 (e.g., to atmosphere). In this way, the fluid handling device 1352 and the cell processing module 1354 can maintain isolation of the chambers 1372, 1374 from the ambient environment, during movement of liquid between the chambers 1372, 1374 (or into one of the chambers 1372, 1374). In some embodiments, the filters 1358, 1368 can each have a pore size of less than or equal to five microns.

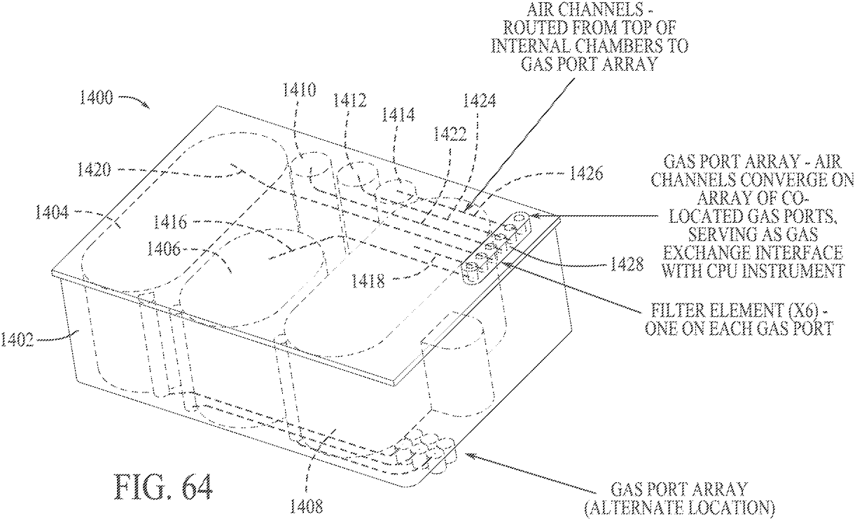

FIG. 64 shows an isometric view of a cell processing module 1400, the description of which is applicable to the other cell processing modules 1400 described herein (and vice versa). The cell processing module 1400 can include a housing 1402, chambers 1404, 1406, 1408, 1410, 1412, 1414 (e.g., each of which can be isolated from the ambient environment), channels 1416, 1418, 1420, 1422, 1424, 1426, and a gas manifold 1248. As shown in FIG. 64, Each of the channels 1416, 1418, 1420, 1422, 1424, 1426 is in fluid communication with the gas manifold (at one end) and in fluid communication with the respective chamber 1406, 1408, 1410, 1412, 1414. In this way, gas from one or more pressure sources can be directed through the gas manifold 1428 to a chamber, thereby driving liquid from the chamber to another chamber (e.g., via adjusting a multi-position valve that fluidically connects the chambers together). In some configurations, each channel 1416, 1418, 1420, 1422, 1424, 1426 can be in fluid communication with a respective filter, upstream (or downstream) of the respective chamber. In this way, gas from the gas manifold flows through the filter before entering the chamber (e.g., to avoid contamination of the liquid within the chamber with contaminants in the gas).

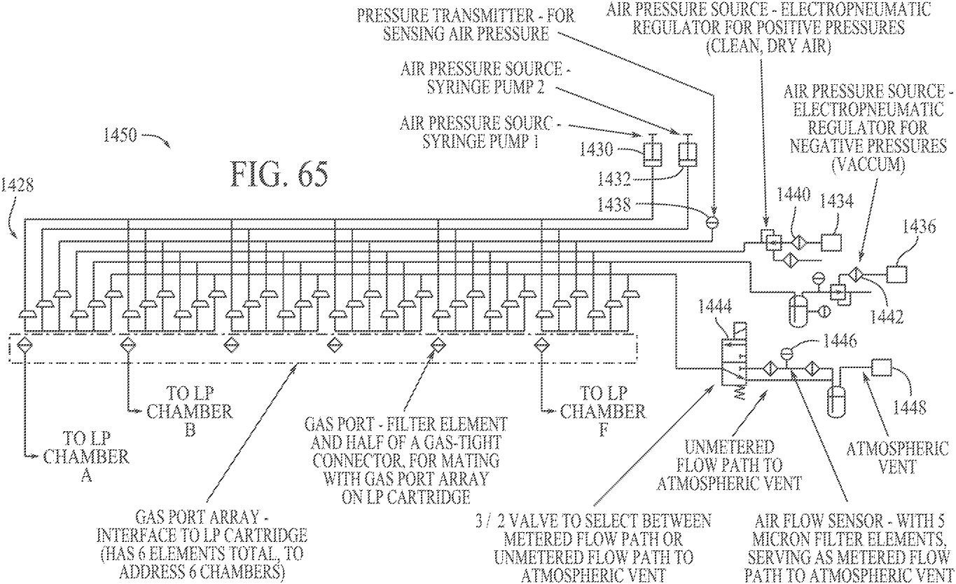

FIG. 65 shows a schematic illustration of the cell processing module 1400, showing the interfacing with pressure sources of a fluid handling device. In some embodiments, the cell processing module 1400 can include pressure sources 1430, 1432, 1434, 1436, each of which can be in fluid communication with the gas manifold 1428. The pressure sources 1430, 1432 can each be a syringe pump, the pressure source 1434 can be a clean gas pressure source (e.g., a medical grade pressure source), and the pressure source 1436 can be a negative pressure source (e.g., to create a vacuum). The cell processing module 1400 can include a pressure sensor 1438 that is in fluid communication with the gas manifold 1428, to, for example, sense a current pressure of gas delivered to the gas manifold 1428 (and to the respective chambers).

In some embodiments, the cell processing module 1400 can include pressure regulators 1440, 1442, each of which can be adjustable (e.g., by a computing device) to adjust the set pressure through the pressure regulator 1440, 1442. For example, each pressure regulator 1440, 1442 can be an electropneumatic pressure regulator, and each pressure regulator 1440, 1442 can be in fluid communication with the respective pressure source 1434, 1436. In some cases, the pressure regulator 1440 can set a positive pressure for gas flowing from the pressure source 1440 through the pressure regulator 1440 and to the gas manifold 1428, while the pressure regulator 1442 can set a negative pressure for gas flowing from the gas manifold 1428 and through the pressure regulator 1442 to the pressure source 1442 (e.g., that is a negative pressure source).

In some embodiments, the cell processing module 1400 can include a multi-position valve 1444, a gas flow sensor 1446, and a vent 1448 (e.g., that is in fluid communication with the ambient environment). The multi-position valve 1444 can have a first position that allows fluid communication between the gas manifold 1428 and the vent 1448 via a first fluid path, a second position that allows fluid communication between the gas manifold 1428 and the vent 1448 via a second fluid path, and a third position that blocks fluid communication between the gas manifold 1428 and the vent 1448. In some cases, the first fluid path can be subjected to sensing of the gas flow by the gas flow sensor 1446, while the second path is not subjected to sensing of the gas flow by the gas flow sensor 1446. In some cases, a computing device (e.g., of the fluid handling device) can be in communication with the pressure sensor 1438, the gas flow sensor 1446, the pressure sources 1430, 1432, 1434, 1436, the pressure regulators 1440, 1442, the multi-position valve 1444, etc.

While the description above has described fluid moving through (and between components), components actuating, etc., in some embodiments, all of the processes described herein can be implemented by one or more computing devices (e.g., of a fluid handling device), as appropriate. For example, the one or more computing devices can cause actuators to move bring components into fluid communication with each other, can cause fluid to flow between components, etc.

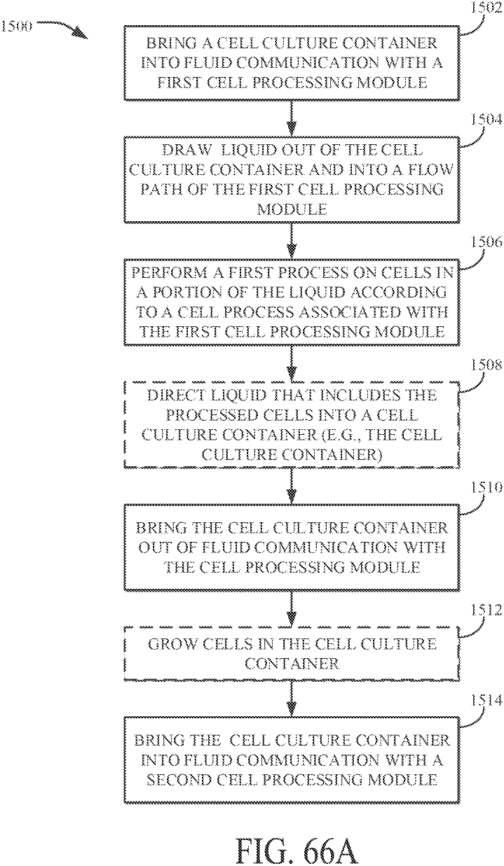

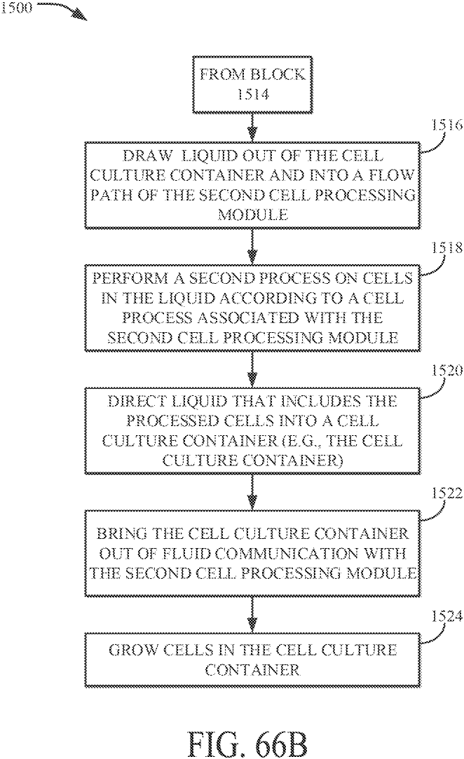

FIGS. 66A and 66B collectively show a flowchart of a process 1500 for processing cells, which can be implemented using any of the cell processing systems (and corresponding components). Similarly, some or all blocks of the process 1500 can be implemented using one or more computing devices, as appropriate, but will reference mainly the corresponding computing device of a cell processing system. In some embodiments, the internal volume of the cell culture container (e.g., the liquid within the internal volume) can be isolated from the ambient environment during some or all blocks of the process 1500. Correspondingly, a flow path of each cell processing module that is brought into (or out of) fluid communication with the internal volume of the cell culture container (e.g., the liquid within the flow path) can be isolated from the ambient environment during some or all blocks of the process 1500. In some embodiments, processing cells can include growing cells (e.g., multiplying cells).

At 1502, the process 1500 can include a computing device brining a cell culture container into fluid communication with a first cell processing module. In some embodiments, the block 1502 can include a computing device aligning a flow coupler with a port of a cell culture container, and bringing a conduit of the flow coupler into fluid communication with the internal volume of the cell culture container, via the port of the cell culture container. For example, this can include a computing device advancing the flow coupler (e.g., by extending an actuator) until the flow coupler is inserted into the internal volume of the cell culture container. In some cases, this can include a computing device opening a barrier of the port of the cell culture container by advancing a reciprocating member of the flow coupler (e.g., by extending an actuator) until the reciprocating member (or a hollow tube coupled thereto) pierces through the barrier (or otherwise opens the barrier) and enters into the internal volume of the cell culture container. In some embodiments, this can include a computing device biasing a spring of the flow coupler, when the flow coupler is advanced towards the cell culture container. In some cases, the block 1502 can be used to bring multiple flow couplers into fluid communication with the internal volume of the cell culture container, via multiple respective ports of the cell culture container.

At 1504, the process 1500 can include a computing device drawing liquid out of the cell culture container and into a flow path of the first cell processing module, which can be isolated from the ambient environment (e.g., liquid positioned within the flow path is blocked from entering the ambient environment). In some cases, this can include a computing device drawing liquid out through the internal volume of the cell culture container (e.g., by activating a pump of a liquid handling device), through the port of the cell culture container, through a conduit of the flow coupler, and into (and through) the flow path of the first cell processing module. In some cases, this can include a computing device drawing gas that is within the internal volume of the cell culture container through a port of the cell culture container, which can occur while liquid is drawing out of the cell culture container.

At 1506, the process 1500 can include a computing device performing a first process on cells in a portion of the liquid according to a cell process associated with the first cell processing module. In some cases, each cell processing module can have one or more cell processes associated therewith, while in other cases, each cell processing module can have a single cell process associated therewith. In some cases, including when the cell processing module has the single cell process associated therewith, the single cell process can be unique to the respective cell processing module. In other words, multiple cell processing modules can each have a single unique cell process associated therewith. In some cases, the block 1506 can include a computing device directing the portion of the liquid through the flow path of the first cell processing module, and implementing the cell process on the cells as the cells pass through the flow path of the cell processing module. In some configurations, the cell process can be cell separation, cell collection, cell transfection, cell electroporation, cell nucleofection, cell lipofection, cell poration, cell harvesting, reagent exchange, reagent removal, or cell sampling. In some configurations, when each cell processing module only includes a single cell process associated therewith, the constructing of each cell processing module can advantageously be constructed in a more simple manner. For example, in this case, the cell processing modules do not need to include multi-position valves to route fluid flow through multiple cell processes compartments of the cell processing module.

At 1508, the process 1500 can include a computing device directing liquid that includes the processed cells (according to the cell process of the first cell processing module) into a cell culture container. In some cases, this cell culture container can be the same cell culture container (e.g., as in the block 1502), while in other cases, this can include another cell culture container. In some cases, using another cell culture container (e.g., rather than the cell culture container) can be advantageous in that the another cell culture container can be free of contaminants. In some cases, this can include a computing device activating a pump to direct the liquid that includes the processed cells from the flow path of the cell processing module, through the conduit of the flow coupler, through the port of the cell culture container, and into the interior volume of the cell culture container.

At 1510, the process 1500 can include a computing device bringing the cell culture container (e.g., of the block 1508) out of fluid communication with the cell processing module. In some cases, this can include a computing device retreating the flow coupler out of the internal volume of the cell culture container. For example, a computing device can cause an actuator to retract, thereby unloading the spring to cause the reciprocating member of the flow coupler to move away from the cell culture container, which can move a hollow tube (or the reciprocating member) out of the internal volume of the cell culture container. In some cases, after the cell culture container is brought out of fluid communication with the cell processing module, the cell culture container can be isolated from the ambient environment (e.g., the liquid within the internal volume can be isolated from the ambient environment). For example, a barrier of the cell culture container can reseal, following removal of the flow coupler from the cell culture container.

At 1512, the process 1500 can include growing cells in the cell culture container (e.g., of the blocks 1508, 1510). In some cases, this can include placing the cell culture container into an incubator. In some cases, growing cells in the cell culture container can include multiplying the cells in the cell culture container.

At 1514, the process 1500 can include a computing device bringing the cell culture container (e.g., of the blocks 1508, 1510, 1512) into fluid communication with a second cell processing module (e.g., a flow path of the cell processing module), which can be similar to the block 1502 of the process 1500. In some embodiments, a cell process associated with the second cell processing module can be different than the cell process of the first cell processing module. In addition, the cell process of the second cell processing module can be the only cell process that the second cell processing module is configured to implement on cells that are positioned within the second cell processing module.

At 1516, the process 1500 can include a computing device drawing liquid out of the cell culture container and into a flow path of the second cell processing module, which can be similar to the block 1504.

At 1518, the process 1500 can include a computing device performing a second process on cells in the liquid (e.g., from the block 1516) according to the cell process associated with the second cell processing module, which can be similar to the block 1506.

At 1520, the process 1500 can include a computing device directing the liquid that includes the processed cells (e.g., according to the cell process of the second cell processing module), into a cell culture container (e.g., the cell culture container of the block 1502, the another cell culture container, or yet another cell culture container).

At 1522, the process 1500 can include a computing device bringing the cell culture container (e.g., of the block 1520) out of fluid communication with the second cell processing module, which can be similar to the block 1510.

At 1524, the process 1500 can include growing cells in the cell culture container, which can be similar to the block 1512.

### ILLUSTRATIVE EMBODIMENTS

The following Embodiments are illustrative and should not be interpreted to limit the scope of the claimed subject matter.

Embodiment 1. A system for processing cells comprising:
(a) a cell culture container;
(b) a fluid handling device;
(c) one or more removable cell processing modules for performing one or more cell processing processes, wherein the one or more removable cell processing modules comprises a fluid handling pathway; and
   wherein the one or more removable cell processing modules are fluidly connected to the cell culture container and the fluid handling device, and
   wherein the system for processing cells is a closed system.

Embodiment 2. The system of embodiment 1, further comprising one or more removable receptacles for receiving the cell culture container and the one or more removable cell processing modules, wherein the one or more removable receptacles connects the cell culture container with the one or more removable cell processing modules.

Embodiment 3. The system of embodiment 1 or 2, wherein only one removable cell processing module of the one or more removable cell processing modules is connected to the cell culture container and the fluid handling device at a time.

Embodiment 4. The system of any one of embodiments 1 to 3, wherein the cell culture container is not directly connected to the fluid handling device.

Embodiment 5. The system of any one of embodiments 1 to 3, wherein the cell culture container is directly connected to the fluid handling device.

Embodiment 6. The system of any one of embodiments 1 to 5, wherein the cell processing process is cell separation.

Embodiment 7. The system of embodiment 6, wherein the removable cell processing module comprises a cell separation device comprising one or more of: a chamber for separating cells, a pressure chamber, a column, a reagent chamber and a waste chamber.

Embodiment 8. The system of embodiment 6 or 7, wherein the removable cell processing module comprises one or more components for separating cells via an antibody, an aptamer, magnetic separation, fluorophore separation, size-based separation, an electric field, centrifugation, sedimentation, flow separation, acoustic separation, filtration or any combination thereof.

Embodiment 9. The system of embodiment 8, wherein the removable cell processing module comprises one or more components for separating cells using an antibody.

Embodiment 10. The system of embodiment 8, wherein the removable cell processing module comprises one or more components for separating cells using an antibody.

Embodiment 11. The system of embodiment 8, wherein the removable cell processing module comprises one or more components for separating cells using an aptamer.

Embodiment 12. The system of any one of embodiments 1 to 11, wherein the cell processing process is cell collection.

Embodiment 13. The system of embodiment 12, wherein the removable cell processing module comprises a cell collection device comprising one or more of: a chamber for collecting cells, a pressure chamber, a column, a reagent chamber and a waste chamber.

Embodiment 14. The system of embodiment 12 or 13, wherein the removable cell processing module comprises one or more components for performing cell collection via centrifugation, sedimentation, flow separation, acoustic separation, filtration, using an antibody, using an aptamer, magnetic separation, fluorophore separation, size-based separation, an electric field or any combination thereof.

Embodiment 15. The system of embodiment 14, wherein the removable cell processing module comprises one or more components for performing cell collection via centrifugation.

Embodiment 16. The system of any one of embodiments 1 to 15, further comprising a centrifugation container for performing centrifugation.

Embodiment 17. The system of embodiment 16, wherein the centrifugation container cannot be used for growing cells.

Embodiment 18. The system of embodiment 16, wherein the centrifugation container can be used for growing cells.

Embodiment 19. The system of any one of embodiments 1 to 18, wherein the removable cell processing module is configured to add beads to a cell culture that is processed in the system.

Embodiment 20. The system of any one of embodiments 1 to 19, wherein the removable cell processing module is configured to remove beads from a cell culture that is processed in the system.

Embodiment 21. The system of any one of embodiments 1 to 20, wherein the removable cell processing module is configured to add beads to a cell culture that is processed in the system and to remove beads from a cell culture that is processed in the system.

Embodiment 22. The system of any one of embodiments 1 to 21, wherein the removable cell processing module comprises one or more of: a magnetic chamber, a pressure chamber and a column.

Embodiment 23. The system of any one of embodiments 1 to 22, wherein the removable cell processing module is configured to perform cell transfection.

Embodiment 24. The system of embodiment 23, wherein the removable cell processing module comprises a cell transfection device comprising a chamber for transfecting cells.

Embodiment 25. The system of embodiment 23 or 24, wherein the removable cell processing module is configured for performing electroporation, nucleofection, lipofection, viral transfection, chemical transfection, mechanical transfection, laser-induced photoporation, needle-based poration, impalefection, magnetofection or sonoporation or any combination thereof.

Embodiment 26. The system of embodiment 25, wherein the removable cell processing module is configured for performing cell transfection via electroporation.

Embodiment 27. The system of embodiment 25, wherein the removable cell processing module is configured for performing cell transfection via nucleofection.

Embodiment 28. The system of any one of embodiments 1 to 27, wherein the removable cell processing module is configured for adding, removing and/or exchanging one or more reagents.

Embodiment 29. The system of any one of embodiments 1 to 28, wherein the removable cell processing module comprises one or more of: a reagent chamber, a pressure chamber and a waste chamber.

Embodiment 30. The system of any one of embodiments 1 to 29, wherein the removable cell processing module is configured for performing sampling.

Embodiment 31. The system of any one of embodiments 1 to 30, wherein the removable cell processing module is configured for use in performing cryopreservation of a cell culture.

Embodiment 32. The system of embodiment 31, wherein the removable cell processing module comprises a cell storage container for use during cryopreservation.

Embodiment 33. The system of embodiment 32, wherein the cell storage container is a bag-based cell storage container comprising one or more fluoropolymer membrane chambers for storing cells.

Embodiment 34. The system of embodiment 33, wherein the cell storage container is a bag-based cell storage container comprising one fluoropolymer membrane chamber for storing cells.

Embodiment 35. The system of embodiment 33, wherein the cell storage container is a bag-based cell storage container comprising two fluoropolymer membrane chambers for storing cells.

Embodiment 36. The system of embodiment 35, wherein the two fluoropolymer membrane chambers for storing cells are connected.

Embodiment 37. The system of any one of embodiments 33 to 36, wherein the one or more fluoropolymer membrane chambers are expandable.

Embodiment 38. The system of any one of embodiments 33 to 37, wherein the one or more fluoropolymer membrane chambers comprise a non-fluoropolymer base.

Embodiment 39. The system of embodiment 38, wherein the one or more fluoropolymer membrane chambers share the same non-fluoropolymer base.

Embodiment 40. The system of embodiment 38 or 39, wherein the non-fluoropolymer base comprises a plastic base.

Embodiment 41. The system of embodiment 40, wherein the plastic base is a polycarbonate base or a polypropylene base.

Embodiment 42. The system of any one of embodiments 33 to 41, wherein the bag-based cell storage container comprises an inlet port and an outlet port.

Embodiment 43. The system of embodiment 42, wherein the inlet port and/or the outlet port comprise self-sterilizing connections.

Embodiment 44. The system of embodiment 42 or 43, wherein the inlet port and the outlet port are the same port.

Embodiment 45. The system of embodiment 42 or 43, wherein the inlet port and the outlet port are different ports.

Embodiment 46. The system of any one of embodiments 1 to 45, wherein the cell culture container is a bag-based cell culture container comprising one or more gas-permeable silicone membrane chambers for processing cells.

Embodiment 47. The system of embodiment 46, wherein the bag-based cell culture container comprises one gas-permeable silicone membrane chambers for processing cells.

Embodiment 48. The system of embodiment 46, wherein the bag-based cell culture container comprises two gas-permeable silicone membrane chambers for processing cells.

Embodiment 49. The system of embodiment 47, wherein the two gas-permeable silicone membrane chambers for processing cells are connected.

Embodiment 50. The system of any one of embodiments 46 to 49, wherein the one or more gas-permeable silicone membrane chambers are expandable.

Embodiment 51. The system of any one of embodiments 46 to 50, wherein the one or more gas-permeable silicone membrane chambers comprise a non-silicone base.

Embodiment 52. The system of embodiment 51, wherein the one or more gas-permeable silicone membrane chambers share the same non-silicone base.

Embodiment 53. The system of embodiment 51 or 52, wherein the non-silicone base comprises a plastic base.

Embodiment 54. The system of embodiment 53, wherein the plastic base is a polycarbonate base or a polypropylene base.

Embodiment 55. The system of any one of embodiments 46 to 54, wherein the bag-based cell culture container comprises an inlet port, an outlet port, and a sampling port.

Embodiment 56. The system of embodiment 55, wherein the inlet port, the outlet port and/or the sampling port comprise self-sterilizing connections.

Embodiment 57. The system of embodiment 55 or 56, wherein the inlet port, the outlet port, and the sampling port are the same port.

Embodiment 58. The system of embodiment 55 or 56, wherein the inlet port, the outlet port, and the sampling port are different ports.

Embodiment 59. The system of any one of embodiments 1 to 58, wherein the cells are cultured in the cell culture container.

Embodiment 60. The system of embodiment 59, wherein the cells are cultured in the one or more gas-permeable silicone membrane chambers of the bag-based cell culture container.

Embodiment 61. The system of any one of embodiments 1 to 60, wherein the system is configured for processing immune cells.

Embodiment 62. The system of embodiment 61, wherein the immune cells are antigen presenting cells.

Embodiment 63. The system of embodiment 61, wherein the immune cells are T-cells.

Embodiment 64. The system of embodiment 61, wherein the immune cells are B-cells.

Embodiment 65. The system of embodiment 61, wherein the immune cells are NK-cells.

Embodiment 66. The system of any one of embodiments 61 to 65, wherein the system is configured for activating the immune cells in the cell culture container.

Embodiment 67. The system of embodiment 66, wherein the system is configured for activating the immune cells in the one or more gas-permeable silicone membrane chambers of the bag-based cell culture container.

Embodiment 68. The system of any one of embodiments 1 to 60, wherein the system is configured for processing stem cells.

Embodiment 69. The system of embodiment 68, wherein the stem cells are hematopoietic stem cells.

Embodiment 70. The system of embodiment 68, wherein the stem cells are mesenchymal stem cells, neural stem cells, epithelial stem cells or embryonic stem cells.

Embodiment 71. The system of embodiment 68, wherein the stem cells are induced pluripotent stem cells.

Embodiment 72. The system of any one of embodiments 68 to 71, wherein the system is configured for differentiating the stem cells in the cell culture container.

Embodiment 73. The system of embodiment 72, wherein the system is configured for differentiating the stem cells in the one or more gas-permeable silicone membrane chambers of the bag-based cell culture container.

Embodiment 74. The system of any one of embodiments 1 to 73, wherein the cells are autologous cells.

Embodiment 75. The system of any one of embodiments 1 to 73, wherein the cells are allogeneic cells.

Embodiment 76. The system of any one of embodiments 1 to 75, wherein the system is configured for processing cells that have been thawed from a frozen state.

Embodiment 77. The system of any one of embodiments 1 to 75, wherein the system is configured for processing cells that have not been frozen and thawed.

Embodiment 78. The system of any one of embodiments 1 to 77, further comprising a self-sterilizing connection between the removable cell processing module and the cell culture container.

Embodiment 79. The system of embodiment 78, wherein the self-sterilizing connection comprises:
(a) a sterile inner cavity;
(b) a sterile first barrier sealing the inner cavity;
(c) a sterile needle in the inner cavity, wherein the needle comprises an inner channel; and
(d) a second barrier sealing a sterile inner lumen;
   wherein the inner cavity, the first barrier and the needle are comprised in the receptacle, and wherein the second barrier and the inner lumen are comprised in the cell culture container;
   wherein the second barrier is exposed to a sterilization agent, and wherein the second barrier is aligned with the first barrier and an actuation force is applied to drive the needle of the removable cell processing module through both barriers to make a sterile connection with the inner lumen of the cell culture container.

Embodiment 80. The system of embodiment 78, wherein the barrier is a septum.

Embodiment 81. The system of any one of embodiments 78 to 80, wherein the self-sterilizing connection is connected to a source of the sterilizing agent.

Embodiment 82. The system of any one of embodiments 79 to 81, wherein the sterilizing agent is hydrogen peroxide, isopropyl alcohol, sterile distilled water, a catalase solution, a hydrogen peroxidase solution, or a gas.

Embodiment 83. The system of embodiment 82, wherein the sterilizing agent is hydrogen peroxide.

Embodiment 84. The system of embodiment 82, wherein the sterilizing agent is isopropyl alcohol.

Embodiment 85. The system of embodiment 84, wherein the sterilizing agent is 70% isopropyl alcohol.

Embodiment 86. The system of any one of embodiments 79 to 85, wherein the actuation force is mechanical.

Embodiment 87. The system of any one of embodiments 79 to 85, wherein the actuation force is pneumatic.

Embodiment 88. The system of any one of embodiments 79 to 85, wherein the actuation force is electrical.

Embodiment 89. The system of any one of embodiments 1 to 88, wherein the system comprises a plurality of removable cell processing modules for performing a cell processing process, and wherein the plurality of modules is selected from the group comprising: a removable cell processing module for performing cell separation, a removable cell processing module for performing cell collection, a removable cell processing module for addition of beads, a removable cell processing module for removal of beads, a removable cell processing module for adding, removing and/or exchanging one or more reagents, a removable cell processing module for performing transfection, a removable cell processing module for performing sampling, and a removable cell processing module for performing cryopreservation.

Embodiment 90. The system of embodiment 89, wherein the system comprises a removable cell processing module for performing cell separation and a removable cell processing module for adding, removing and/or exchanging one or more reagents.

Embodiment 91. The system of embodiment 90, wherein the system further comprises a removable cell processing module for performing cell collection.

Embodiment 92. The system of embodiment 90 or 91, wherein the system further comprises a removable cell processing module for addition of beads and a removable cell processing module for removal of beads.

Embodiment 93. The system of embodiment 90 or 91, wherein the system further comprises a removable cell processing module for addition and/or removal of beads.

Embodiment 94. The system of any one of embodiments 90 to 93, wherein the system further comprises a removable cell processing module for transfection.

Embodiment 95. The system of any one of embodiments 90 to 94, wherein the system further comprises a removable cell processing module for cryopreservation.

Embodiment 96. The system of any one of embodiments 90 to 95, wherein the system further comprises a removable cell processing module for sampling.

Embodiment 97. The system of any one of embodiments 1 to 96, wherein the system comprises a removable cell processing module for obtaining cells from a subject.

Embodiment 98. The system of any one of embodiments 1 to 97, wherein the system comprises a removable cell processing module for administering cells to a subject.

Embodiment 99. The system of embodiment 97 or 98, wherein the subject is a mammal.

Embodiment 100. The system of embodiment 99, wherein the subject is human.

Embodiment 101. The system of any one of embodiments 1 to 99, wherein the system further comprises a removable cell processing module dispenser to dispense the one or more removable cell processing modules.

Embodiment 102. The system of embodiment 101, wherein the removable cell processing module dispenser dispenses the one or more removable cell processing modules to the removable receptacle for receiving the one or more removable cell processing modules.

Embodiment 103. The system of any one of embodiments 1 to 102, wherein the system further comprises a cell collection device to perform cell collection.

Embodiment 104. The system of embodiment 103, wherein the cell collection is performed via centrifugation, sedimentation, flow separation, acoustic separation, filtration, using an antibody, using an aptamer, magnetic separation, fluorophore separation, size-based separation, an electric field or any combination thereof.

Embodiment 105. The system of embodiment 104, wherein the cell collection is performed via centrifugation.

Embodiment 106. The system of any one of embodiments 1 to 105, wherein the system further comprises a reagent source.

Embodiment 107. The system of any one of embodiments 1 to 106, wherein the system further comprises an incubator.

Embodiment 108. The system of any one of embodiments 1 to 107, wherein the system further comprises a sampling device.

Embodiment 109. The system of any one of embodiments 1 to 108, wherein the system further comprises an analytical device.

Embodiment 110. The system of embodiment 109, wherein the analytical device is an imaging device.

Embodiment 111. The system of embodiment 109 or 110, wherein the analytical device is a spectrometry device.

Embodiment 112. The system of any one of embodiments 1 to 111, wherein the system further comprises a robotic arm to transport the cell culture container and/or the one or more removable cell processing module to one or more of: the fluid handling device, a removable cell processing module dispenser, a cell collection device, a reagent source, an incubator, a mixer, a sampling device and a removable cell processing module dispenser.

Embodiment 113. The system of any one of embodiments 1 to 112, wherein the system is an automated system.

Embodiment 114. The system of any one of embodiments 1 to 113, wherein the system is enclosed in a housing.

Embodiment 115. A system for processing cells comprising a plurality of systems of any one of embodiments 1 to 114, wherein the plurality of systems is capable of processing cells in parallel.

Embodiment 116. The system of embodiment 115, wherein the plurality of systems is a plurality of stackable systems.

Embodiment 117. The system of any one of embodiments 1 to 116, wherein the system is a point-of-care system.

Embodiment 118. The system of embodiment 117, wherein the point-of-care system is a bedside system.

Embodiment 119. The system of any one of embodiments 1 to 118, wherein the system is operated in a sterile environment.

Embodiment 120. A method of processing cells comprising:
(a) growing or incubating cells in a cell culture container;
(b) passing the cells and/or one or more reagents through one or more removable cell processing modules and performing a cell processing process in the one or more removable cell processing modules, wherein the one or more removable cell processing modules comprises a fluid handling pathway; and
(c) a fluid handling device for handling fluids;
   wherein the one or more removable cell processing modules is connected to the cell culture container and the fluid handling device, and
wherein the processing of cells is carried out in a closed system.

Embodiment 121. The method of embodiment 120, wherein the system further comprises one or more removable receptacles, wherein the one or more removable receptacle connects the cell culture container with the one or more removable cell processing modules.

Embodiment 122. The method of embodiment 120 or 121, wherein only one removable cell processing module of the one or more removable cell processing modules can be connected to the cell culture container and the fluid handling device at a time.

Embodiment 123. The method of any one of embodiments 120 to 122, wherein the cell culture container is not directly connected to the fluid handling device.

Embodiment 124. The method of any one of embodiments 120 to 122, wherein the cell culture container is directly connected to the fluid handling device.

Embodiment 125. The method of any one of embodiments 120 to 124, wherein the cell processing process is cell separation.

Embodiment 126. The method of embodiment 125, wherein the removable cell processing module for performing cell separation comprises a cell separation device comprising one or more of: a chamber for separating cells, a pressure chamber, a column, a reagent chamber and a waste chamber.

Embodiment 127. The method of embodiment 125 or 126, wherein the cells are separated using an antibody, an aptamer, magnetic separation, fluorophore separation, size-based separation, an electric field, centrifugation, sedimentation, flow separation, acoustic separation, filtration or any combination thereof.

Embodiment 128. The method of embodiment 127, wherein the cells are separated using an antibody.

Embodiment 129. The method of embodiment 127, wherein the cells are separated using an aptamer.

Embodiment 130. The method of any one of embodiments 120 to 129, wherein the cell processing process is cell collection.

Embodiment 131. The method of embodiment 130, wherein the removable cell processing module for performing cell collection comprises a cell collection device comprising one or more of: a chamber for collecting cells, a pressure chamber, a column, a reagent chamber and a waste chamber.

Embodiment 132. The method of embodiment 130 or 131, wherein the cells are collected via centrifugation, sedimentation, flow separation, acoustic separation, filtration, using an antibody, using an aptamer, magnetic separation, fluorophore separation, size-based separation, an electric field or any combination thereof.

Embodiment 133. The method of embodiment 132, wherein the cells are collected via centrifugation.

Embodiment 134. The method of embodiment 132 or 133, wherein the cells are collected via centrifugation in a centrifugation container.

Embodiment 135. The method of embodiment 134, wherein the centrifugation container cannot be used for growing cells.

Embodiment 136. The system of embodiment 134, wherein the centrifugation container can be used for growing cells.

Embodiment 137. The method of any one of embodiments 120 to 136, wherein the cell processing process is addition of beads.

Embodiment 138. The method of any one of embodiments 120 to 137, wherein the cell processing process is removal of beads.

Embodiment 139. The method of any one of embodiments 120 to 138, wherein the wherein the cell processing process is addition and/or removal of beads.

Embodiment 140. The method of any one of embodiments 120 to 139, wherein the removable cell processing module comprises one or more of: a magnetic chamber, a pressure chamber and a column.

Embodiment 141. The method of any one of embodiments 120 to 140, wherein the cell processing process is cell transfection.

Embodiment 142. The method of embodiment 141, wherein the removable cell processing module for performing cell transfection comprises a cell transfection device comprising a chamber for transfecting cells.

Embodiment 143. The method of embodiment 141 or 142, wherein the cell transfection is performed via electroporation, nucleofection, lipofection, viral transfection, chemical transfection, mechanical transfection, laser-induced photoporation, needle-based poration, impalefection, magnetofection or sonoporation or any combination thereof.

Embodiment 144. The method of embodiment 143, wherein the cell transfection is performed via electroporation.

Embodiment 145. The method of embodiment 143, wherein the cell transfection is performed via nucleofection.

Embodiment 146. The method of any one of embodiments 120 to 145, wherein the cell processing process is adding, removing and/or exchanging one or more reagents.

Embodiment 147. The method of any one of embodiments 120 to 146, wherein the removable cell processing module comprises one or more of: a reagent chamber, a pressure chamber and a waste chamber.

Embodiment 148. The method of any one of embodiments 120 to 147, wherein the cell processing process is sampling.

Embodiment 149. The method of any one of embodiments 120 to 148, wherein the cell processing process is cryopreservation.

Embodiment 150. The method of embodiment 149, wherein the removable cell processing module for performing cryopreservation comprises a cell storage container.

Embodiment 151. The method of embodiment 150, wherein the cell storage container is a bag-based cell storage container comprising one or more fluoropolymer membrane chambers for storing cells.

Embodiment 152. The method of embodiment 151, wherein the cell storage container is a bag-based cell storage container comprising one fluoropolymer membrane chambers for storing cells.

Embodiment 153. The method of embodiment 151, wherein the cell storage container is a bag-based cell storage container comprising two fluoropolymer membrane chambers for storing cells.

Embodiment 154. The method of embodiment 153, wherein the two gas-permeable silicone membrane chambers for storing cells are connected.

Embodiment 155. The method of any one of embodiments 151 to 154, wherein the one or more fluoropolymer membrane chambers are expandable.

Embodiment 156. The method of any one of embodiments 151 to 155, wherein the one or more fluoropolymer membrane chambers comprise a non-fluoropolymer base.

Embodiment 157. The method of embodiment 156, wherein the one or more fluoropolymer membrane chambers share the same non-fluoropolymer base.

Embodiment 158. The method of embodiment 156 or 157, wherein the non-fluoropolymer base comprises a plastic base.

Embodiment 159. The method of embodiment 158, wherein the plastic base is a polycarbonate base or a polypropylene base.

Embodiment 160. The method of any one of embodiments 151 to 159, wherein the bag-based cell storage container comprises an inlet port and an outlet port.

Embodiment 161. The method of embodiment 160, wherein the inlet port and/or the outlet port comprise self-sterilizing connections.

Embodiment 162. The method of any one of embodiments 120 to 161, wherein the cell culture container is a bag-based cell culture container comprising one or more gas-permeable silicone membrane chambers for processing cells.

Embodiment 163. The method of embodiment 162, wherein the bag-based cell culture container comprises one gas-permeable silicone membrane chambers for processing cells.

Embodiment 164. The method of embodiment 162, wherein the bag-based cell culture container comprises two gas-permeable silicone membrane chambers for processing cells.

Embodiment 165. The method of embodiment 164, wherein the two gas-permeable silicone membrane chambers for processing cells are connected.

Embodiment 166. The method of any one of embodiments 161 to 165, wherein the one or more gas-permeable silicone membrane chambers are expandable.

Embodiment 167. The method of any one of embodiments 161 to 166, wherein the one or more gas-permeable silicone membrane chambers comprise a non-silicone base.

Embodiment 168. The method of embodiment 167, wherein the one or more gas-permeable silicone membrane chambers share the same non-silicone base.

Embodiment 169. The method of embodiment 167 or 168, wherein the non-silicone base comprises a plastic base.

Embodiment 170. The method of embodiment 169, wherein the plastic base is a polycarbonate base or a polypropylene base.

Embodiment 171. The method of any one of embodiments 161 to 170, wherein the bag-based cell culture container comprises an inlet port, an outlet port and a sampling port.

Embodiment 172. The method of embodiment 171, wherein the inlet port, the outlet port and/or the sampling port comprise self-sterilizing connections.

Embodiment 173. The method of embodiment 171 or 172, wherein the inlet port and the outlet port are the same port.

Embodiment 174. The method of embodiment 171 or 172, wherein the inlet port and the outlet port are different ports.

Embodiment 175. The method of any one of embodiments 161 to 174, wherein the cells are cultured in the bag-based cell culture container.

Embodiment 176. The method of embodiment 175, wherein the cells are cultured in the one or more gas-permeable silicone membrane chambers of the bag-based cell culture container.

Embodiment 177. The method of any one of embodiments 120 to 176, wherein the cells are immune cells.

Embodiment 178. The method of embodiment 177, wherein the immune cells are antigen presenting cells.

Embodiment 179. The method of embodiment 177, wherein the immune cells are T-cells.

Embodiment 180. The method of embodiment 177, wherein the immune cells are B-cells.

Embodiment 181. The method of embodiment 177, wherein the immune cells are NK-cells.

Embodiment 182. The method of any one of embodiments 177 to 181, wherein the immune cells are activated in the bag-based cell culture container.

Embodiment 183. The method of embodiment 182, wherein the immune cells are activated in the one or more gas-permeable silicone membrane chambers of the bag-based cell culture container.

Embodiment 184. The method of any one of embodiments 120 to 176, wherein the cells are stem cells.

Embodiment 185. The method of embodiment 184, wherein the stem cells are hematopoietic stem cells.

Embodiment 186. The method of embodiment 184, wherein the stem cells are mesenchymal stem cells, neural stem cells, epithelial stem cells or embryonic stem cells.

Embodiment 187. The method of embodiment 184, wherein the stem cells are induced pluripotent stem cells.

Embodiment 188. The method of any one of embodiments 184 to 187, wherein the stem cells are differentiated in the bag-based cell culture container.

Embodiment 189. The method of embodiment 188, wherein the stem cells are differentiated in the one or more gas-permeable silicone membrane chambers of the bag-based cell culture container.

Embodiment 190. The method of any one of embodiments 120 to 189, wherein the cells are autologous cells.

Embodiment 191. The method of any one of embodiments 120 to 189, wherein the cells are allogeneic cells.

Embodiment 192. The method of any one of embodiments 120 to 191, wherein the cells are thawed from a frozen state.

Embodiment 193. The method of any one of embodiments 120 to 191, wherein the cells have not been frozen and thawed.

Embodiment 194. The method of any one of embodiments 120 to 193, wherein a connection between the removable cell processing module and the cell culture container is via a self-sterilizing connection.

Embodiment 195. The method of embodiment 194, wherein the self-sterilizing connection comprises:
(a) a sterile inner cavity;
(b) a sterile first barrier sealing the inner cavity;
(c) a sterile needle in the inner cavity, wherein the needle comprises an inner channel; and
(d) a second barrier sealing a sterile inner lumen,
   wherein the inner cavity, the first barrier and the needle are comprised in the removable cell processing module, and wherein the second barrier and the inner lumen are comprised in the cell culture container,
   wherein the method comprises exposing the second barrier to a sterilization agent, aligning the second barrier with the first barrier and applying an actuation force to drive the needle of the removable cell processing module through both barriers to make a sterile connection with the inner lumen of the cell culture container.

Embodiment 196. The method of embodiment 195, wherein the barrier is a septum.

Embodiment 197. The method of any one of embodiments 194 to 196, wherein the self-sterilizing connection is connected to a source of the sterilizing agent.

Embodiment 198. The method of any one of embodiments 195 to 197, wherein the sterilizing agent is hydrogen peroxide, isopropyl alcohol, sterile distilled water, a catalase solution, a hydrogen peroxidase solution, or a gas.

Embodiment 199. The method of embodiment 198, wherein the sterilizing agent is hydrogen peroxide.

Embodiment 200. The method of embodiment 198, wherein the sterilizing agent is isopropyl alcohol.

Embodiment 201. The method of embodiment 200, wherein the sterilizing agent is 70% isopropyl alcohol.

Embodiment 202. The method of any one of embodiments 195 to 201, wherein the actuation force is mechanical.

Embodiment 203. The method of any one of embodiments 195 to 201, wherein the actuation force is pneumatic.

Embodiment 204. The method of any one of embodiments 195 to 201, wherein the actuation force is electrical.

Embodiment 205. The method of any one of embodiments 120 to 204, wherein the system comprises a plurality of removable cell processing modules for performing a cell processing process, and wherein the plurality of cell processing modules is selected from the group comprising: a removable cell processing module for performing cell separation, a removable cell processing module for performing cell collection, a removable cell processing module for addition of beads, a removable cell processing module for removal of beads, a removable cell processing module for adding, removing and/or exchanging one or more reagents, a removable cell processing module for transfection, a removable cell processing module for sampling, a removable cell processing module for transfection and a removable cell processing module for cryopreservation.

Embodiment 206. The method of embodiment 205, wherein the system comprises a removable cell processing module for performing cell separation and a removable cell processing module for adding, removing and/or exchanging one or more reagents.

Embodiment 207. The method of embodiment 206, wherein the system further comprises a removable cell processing module for performing cell collection.

Embodiment 208. The method of embodiment 206 or 207, wherein the system further comprises a removable cell processing module for addition of beads and a removable cell processing module for removal of beads.

Embodiment 209. The method of embodiment 206 or 207, wherein the system further comprises a removable cell processing module for addition and/or removal of beads.

Embodiment 210. The method of any one of embodiments 195 to 209, wherein the system further comprises a removable cell processing module for transfection.

Embodiment 211. The method of any one of embodiments 195 to 210, wherein the system further comprises a removable cell processing module for cryopreservation.

Embodiment 212. The method of any one of embodiments 195 to 211, wherein the system further comprises a removable cell processing module for sampling.

Embodiment 213. The method of any one of embodiments 120 to 212, wherein the system comprises a removable cell processing module for obtaining cells from a subject and cells are obtained from the subject using the removable cell processing module for obtaining cells.

Embodiment 214. The method of any one of embodiments 120 to 213, wherein the system comprises a removable cell processing module for administering cells to a subject and cells are administered to the subject using the removable cell processing module for administering cells.

Embodiment 215. The method of embodiment 213 or 214, wherein the subject is a mammal.

Embodiment 216. The method of embodiment 215, wherein the subject is human.

Embodiment 217. The method of any one of embodiments 1 to 216, wherein the system further comprises a removable cell processing module dispenser to dispense the one or more removable cell processing module and the one or more removable cell processing module is dispensed via the removable cell processing module dispenser.

Embodiment 218. The method of embodiment 217, wherein the removable cell processing module dispenser dispenses the one or more removable cell processing module connected to the one or more removable receptacle.

Embodiment 219. The method of any one of embodiments 120 to 218, wherein the system further comprises a cell collection device to perform cell collection.

Embodiment 220. The method of embodiment 219, wherein the cell collection is performed via centrifugation, sedimentation, flow separation, acoustic separation, filtration, using an antibody, using an aptamer, magnetic separation, fluorophore separation, size-based separation, an electric field or any combination thereof.

Embodiment 221. The method of embodiment 220, wherein the cell collection is performed via centrifugation.

Embodiment 222. The method of any one of embodiments 120 to 221, wherein the system further comprises a reagent source.

Embodiment 223. The method of any one of embodiments 120 to 222, wherein the system further comprises an incubator and the cells are incubated in the incubator.

Embodiment 224. The method of any one of embodiments 120 to 223, wherein the system further comprises a sampling device and samples of the cells are taken via the sampling device.

Embodiment 225. The method of any one of embodiments 120 to 224, wherein the system further comprises an analytical device and the cells and/or a medium for growing the cells are analyzed via the analytical device.

Embodiment 226. The method of embodiment 225, wherein the analytical device is an imaging device.

Embodiment 227. The method of embodiment 225 or 226, wherein the analytical device is a spectrometry device.

Embodiment 228. The method of any one of embodiments 120 to 227, wherein the system further comprises a robotic arm to transport the cell culture container and/or the one or more removable cell processing module to one or more of: the fluid handling device, a cell collection device, a reagent source, an incubator, a mixer, a sampling device and a removable cell processing module dispenser.

Embodiment 229. The method of any one of embodiments 120 to 228, wherein the system is an automated system.

Embodiment 230. The method of any one of embodiments 120 to 229, wherein the system is enclosed in a housing.

Embodiment 231. A method for processing cells in parallel using a plurality of systems of any one of embodiments 1 to 114.

Embodiment 232. The method of embodiment 231, wherein the plurality of systems is a plurality of stackable systems.

Embodiment 233. The method of any one of embodiments 120 to 232, wherein the system is a point-of-care system.

Embodiment 234. The method of embodiment 233, wherein the point-of-care method is a bedside system.

Embodiment 235. The method of any one of embodiments 120 to 234, wherein the system is operated in a sterile environment.

Embodiment 236. A bag-based cell culture container comprising one or more gas-permeable silicone membrane chambers for processing cells.

Embodiment 237. The bag-based cell culture container of embodiment 236, wherein the container comprises one gas-permeable silicone membrane chambers for processing cells.

Embodiment 238. The bag-based cell culture container of embodiment 236, wherein the container comprises two gas-permeable silicone membrane chambers for processing cells.

Embodiment 239. The bag-based cell culture container of embodiment 238, wherein the two gas-permeable silicone membrane chambers for processing cells are connected.

Embodiment 240. The bag-based cell culture container of any one of embodiments 236 to 239, wherein the one or more gas-permeable silicone membrane chambers are expandable.

Embodiment 241. The bag-based cell culture container of any one of embodiments 236 to 240, wherein the one or more gas-permeable silicone membrane chambers comprise a non-silicone base.

Embodiment 242. The bag-based cell culture container of embodiment 241, wherein the one or more gas-permeable silicone membrane chambers share the same non-silicone base.

Embodiment 243. The bag-based cell culture container of embodiment 241 or 242, wherein the non-silicone base comprises a plastic base.

Embodiment 244. The bag-based cell culture container of embodiment 243, wherein the plastic base is a polycarbonate base or a polypropylene base.

Embodiment 245. The bag-based cell culture container of any one of embodiments 236 to 244, wherein the bag-based cell culture container comprises an inlet port, an outlet port and a sampling port.

Embodiment 246. The bag-based cell culture container of embodiment 245, wherein the inlet port and/or the sampling port comprise a septum.

Embodiment 247. The bag-based cell culture container of any one of embodiments 236 to 246, wherein the cells are cultured in the one or more gas-permeable silicone membrane chambers of the bag-based cell culture container.

Embodiment 248. The bag-based cell culture container of any one of embodiments 236 to 247, wherein the cells are immune cells.

Embodiment 249. The bag-based cell culture container of embodiment 248, wherein the immune cells are T-cells.

Embodiment 250. The bag-based cell culture container of embodiment 248, wherein the immune cells are B-cells.

'Embodiment 251. The bag-based cell culture container of embodiment 248, wherein the immune cells are NK-cells.

Embodiment 252. The bag-based cell culture container of any one of embodiments 248 to 251, wherein the immune cells are activated in the one or more gas-permeable silicone membrane chambers of the bag-based cell culture container.

Embodiment 253. The bag-based cell culture container of any one of embodiments 236 to 247, wherein the cells are stem cells.

Embodiment 254. The bag-based cell culture container of embodiment 253, wherein the stem cells are hematopoietic stem cells.

Embodiment 255. The bag-based cell culture container of embodiment 253, wherein the stem cells are mesenchymal stem cells, neural stem cells, epithelial stem cells or embryonic stem cells.

Embodiment 256. The bag-based cell culture container of embodiment 253, wherein the stem cells are induced pluripotent stem cells.

Embodiment 257. The bag-based cell culture container of any one of embodiments 253 to 256, wherein the stem cells are differentiated in the one or more gas-permeable silicone membrane chambers of the bag-based cell culture container.

Embodiment 258. The bag-based cell culture container of any one of embodiments 236 to 257, wherein the cells are autologous cells.

Embodiment 259. The bag-based cell culture container of any one of embodiments 236 to 257, wherein the cells are allogeneic cells.

Embodiment 260. The bag-based cell culture container of any one of embodiments 236 to 259, wherein the gas-permeable silicone membrane has a flat surface that is prevented from being expanded to be curved.

Embodiment 261. The bag-based cell culture container of embodiment 260, wherein the gas-permeable silicone membrane has a substrate below the surface of said membrane that prevents said membrane from being expanded to be curved.

Embodiment 262. The bag-based cell culture container of embodiment 261, wherein the substrate is a mesh.

Embodiment 263. The bag-based cell culture container of any one of embodiments 236 to 262, wherein the gas-permeable silicone membrane chamber for processing cells is isolated from the ambient environment.

Embodiment 264. A cell culture container comprising:
a frame having an upper piece and a lower piece;
a membrane positioned between the upper piece and the lower piece of the frame, the membrane and the upper piece defining an internal volume of the cell culture container, and the membrane having a flat surface that is prevented from being expanded to be curved.

Embodiment 265. The cell culture container of embodiment 264, wherein the lower piece of the frame includes a substrate, and

wherein the membrane contacts the substrate to define the flat surface.

Embodiment 266. The cell culture container of embodiment 265, wherein the substate includes a mesh.

Embodiment 267. The cell culture container of embodiment 265, wherein the membrane is gas permeable, and
wherein the substrate includes one or more channels that facilitate gas flow between the internal volume of the cell culture container and the ambient environment through the membrane and the one or more channels.

Embodiment 268. The cell culture container of embodiment 265, wherein the membrane is non-expandable.

Embodiment 269. The cell culture container of embodiment 265, wherein the substrate contacts the membrane to block the membrane from expanding beyond the substrate.

Embodiment 270. The cell culture container of embodiment 264, further comprising one or more ports that are in fluid communication with the internal volume of the cell culture container.

Embodiment 271. The cell culture container of embodiment 270, wherein the one or more ports includes at least one of:
a first port that is a gas port, the first port directing gas into or out of the internal volume of the cell culture container through the first port; or
a second port that is a liquid port, the second port directing liquid into or out of the internal volume of the cell culture container through the second port.

Embodiment 272. The cell culture container of embodiment 270, wherein the one or more ports includes the first port, and
wherein at least one of:
   gas is configured to flow through the first port and enter the internal volume of the cell culture container at an upper region of the internal volume of the cell culture container; or
   gas is configured to flow out of the internal volume of the cell culture container at an upper region of the internal volume of the cell culture container and through the first port.

Embodiment 273. The cell culture container of embodiment 272, further comprising a conduit that is in fluid communication with the first port and the internal volume of the cell culture container, and
wherein gas flows through the conduit and through the first port.

Embodiment 274. The cell culture container of embodiment 271, wherein the one or more ports includes the second port, and
wherein at least one of:
   liquid is configured to flow through the second port and enter the internal volume of the cell culture container at a lower region of the internal volume of the cell culture container; or
   liquid is configured to flow out of the internal volume of the cell culture container at the upper region of the internal volume of the cell culture container and through the second port.

Embodiment 275. The cell culture container of embodiment 264, wherein the lower piece of the frame includes a substrate positioned below the membrane, and
wherein the membrane is configured to be drawn towards the upper piece of the frame away from the substrate.

Embodiment 276. The cell culture container of embodiment 264, wherein a peripheral end of the membrane is configured to be positioned between the upper piece and the lower piece fo the frame.

Embodiment 277. The cell culture container of embodiment 264, wherein the internal volume of the cell culture container is isolated from the ambient environment.

Embodiment 278. The cell culture container of embodiment 277, wherein isolated from the ambient environment includes liquid positioned within the internal volume of the cell culture container being blocked from passing into the ambient environment.

Embodiment 279. A method for processing cells in a bag-based cell culture container comprising performing a cell processing process in one or more gas-permeable silicone membrane chambers of the bag-based cell culture container.

Embodiment 280. The method of embodiment 279, wherein the container comprises one gas-permeable silicone membrane chambers for processing cells.

Embodiment 281. The method of embodiment 279, wherein the container comprises two gas-permeable silicone membrane chambers for processing cells.

Embodiment 282. The method of embodiment 281, wherein the two gas-permeable silicone membrane chambers for processing cells are connected.

Embodiment 283. The method of any one of embodiments 279 to 282, wherein the one or more gas-permeable silicone membrane chambers are expandable.

Embodiment 284. The method of any one of embodiments 279 to 283, wherein the one or more gas-permeable silicone membrane chambers comprise a non-silicone base.

Embodiment 285. The method of embodiment 284, wherein the one or more gas-permeable silicone membrane chambers share the same non-silicone base.

Embodiment 286. The method of embodiment 284 or 285, wherein the non-silicone base comprises a plastic base.

Embodiment 287. The method of embodiment 286, wherein the plastic base is a polycarbonate base or a polypropylene base.

Embodiment 288. The method of any one of embodiments 279 to 287, wherein the bag-based cell culture container comprises an inlet port, an outlet port and a sampling port.

Embodiment 289. The method of embodiment 288, wherein the inlet port and/or the sampling port comprise a septum.

Embodiment 290. The method of any one of embodiments 279 to 289, wherein the cells are incubated in the one or more gas-permeable silicone membrane chambers of the bag-based cell culture container.

Embodiment 291. The method of any one of embodiments 279 to 290, wherein the cells are cultured in the one or more gas-permeable silicone membrane chambers of the bag-based cell culture container.

Embodiment 292. The method of any one of embodiments 279 to 291, wherein the cells are immune cells.

Embodiment 293. The method of embodiment 292, wherein the immune cells are T-cells.

Embodiment 294. The method of embodiment 292, wherein the immune cells are B-cells.

Embodiment 295. The method of embodiment 292, wherein the immune cells are NK-cells.

Embodiment 296. The method of any one of embodiments 292 to 295, wherein the immune cells are activated in the one or more gas-permeable silicone membrane chambers of the bag-based cell culture container.

Embodiment 297. The method of any one of embodiments 279 to 291, wherein the cells are stem cells.

Embodiment 298. The method of embodiment 297, wherein the stem cells are hematopoietic stem cells.

Embodiment 299. The method of embodiment 297, wherein the stem cells are mesenchymal stem cells, neural stem cells, epithelial stem cells or embryonic stem cells.

Embodiment 300. The method of embodiment 297, wherein the stem cells are induced pluripotent stem cells.

Embodiment 301. The method of any one of embodiments 297 to 300, wherein the stem cells are differentiated in the one or more gas-permeable silicone membrane chambers of the bag-based cell culture container.

Embodiment 302. The method of any one of embodiments 279 to 301, wherein the cells are autologous cells.

Embodiment 303. The method of any one of embodiments 279 to 301, wherein the cells are allogeneic cells.

Embodiment 304. A bag-based cell storage container comprising one or more fluoropolymer membrane chambers for storing cells, wherein the one or more fluoropolymer membrane chambers comprise a non-fluoropolymer base.

Embodiment 305. The bag-based cell storage container of embodiment 304, wherein the one or more fluoropolymer membrane chambers share the same non-fluoropolymer base.

Embodiment 306. The bag-based cell storage container of embodiment 304 or 305, wherein the non-fluoropolymer base comprises a plastic base.

Embodiment 307. The bag-based cell storage container of embodiment 306, wherein the plastic base is a polycarbonate base or a polypropylene base.

Embodiment 308. The bag-based cell storage container of any one of embodiments 304 to 307, wherein the cell storage container comprises one fluoropolymer membrane chamber for storing cells.

Embodiment 309. The bag-based cell storage container of any one of embodiments 304 to 307, wherein the cell storage container comprises two fluoropolymer membrane chambers for storing cells.

Embodiment 310. The bag-based cell storage container of embodiment 309, wherein the two fluoropolymer membrane chambers for storing cells are connected.

Embodiment 311. The bag-based cell storage container of any one of embodiments 304 to 310, wherein the one or more fluoropolymer membrane chambers are expandable.

Embodiment 312. The bag-based cell storage container of any one of embodiments 304 to 311, wherein the bag-based cell storage container comprises an inlet port and an outlet port.

Embodiment 313. The bag-based cell storage container of embodiment 312, wherein the inlet port and/or the outlet port comprise a septum.

Embodiment 314. The bag-based cell storage container of any one of embodiments 304 to 313, wherein the cells are immune cells.

Embodiment 315. The bag-based cell storage container of embodiment 314, wherein the immune cells are T-cells.

Embodiment 316. The bag-based cell storage container of embodiment 314, wherein the immune cells are B-cells.

Embodiment 317. The bag-based cell storage container of embodiment 314, wherein the immune cells are NK-cells.

Embodiment 318. The bag-based cell storage container of any one of embodiments 304 to 313, wherein the cells are stem cells.

Embodiment 319. The bag-based cell storage container of embodiment 318, wherein the stem cells are hematopoietic stem cells.

Embodiment 320. The bag-based cell storage container of embodiment 318, wherein the stem cells are mesenchymal stem cells, neural stem cells, epithelial stem cells or embryonic stem cells.

Embodiment 321. The bag-based cell storage container of embodiment 318, wherein the stem cells are induced pluripotent stem cells.

Embodiment 322. The bag-based cell storage container of any one of embodiments 304 to 321, wherein the cells are autologous cells.

Embodiment 323. The bag-based cell storage container of any one of embodiments 304 to 321, wherein the cells are allogeneic cells.

Embodiment 324. A method for storing cells in a bag-based cell storage container comprising storing cells in one or more fluoropolymer membrane chambers of the bag-based cell storage container, wherein the one or more fluoropolymer membrane chambers comprise a non-fluoropolymer base.

Embodiment 325. The method of embodiment 324, wherein the one or more fluoropolymer membrane chambers share the same non-fluoropolymer base.

Embodiment 326. The method of embodiment 324 or 325, wherein the non-fluoropolymer base comprises a plastic base.

Embodiment 327. The method of embodiment 326, wherein the plastic base is a polycarbonate base or a polypropylene base.

Embodiment 328. The method of any one of embodiments 324 to 327, wherein the cell storage container comprises one fluoropolymer membrane chamber for storing cells.

Embodiment 329. The method of any one of embodiments 324 to 327, wherein the cell storage container comprises two fluoropolymer membrane chambers for storing cells.

Embodiment 330. The method of embodiment 329, wherein the two fluoropolymer membrane chambers for storing cells are connected.

Embodiment 331. The method of any one of embodiments 324 to 330, wherein the one or more fluoropolymer membrane chambers are expandable.

Embodiment 332. The method of any one of embodiments 324 to 331, wherein the bag-based cell storage container comprises an inlet port and an outlet port.

Embodiment 333. The method of embodiment 332, wherein the inlet port and/or the outlet port comprise a septum.

Embodiment 334. The method of any one of embodiments 324 to 333, wherein the cells are immune cells.

Embodiment 335. The method of embodiment 334, wherein the immune cells are T-cells.

Embodiment 336. The method of embodiment 334, wherein the immune cells are B-cells.

Embodiment 337. The method of embodiment 334, wherein the immune cells are NK-cells.

Embodiment 338. The method of any one of embodiments 324 to 337, wherein the cells are stem cells.

Embodiment 339. The method of embodiment 338, wherein the stem cells are hematopoietic stem cells.

Embodiment 340. The method of embodiment 338, wherein the stem cells are mesenchymal stem cells, neural stem cells, epithelial stem cells or embryonic stem cells.

Embodiment 341. The method of embodiment 338, wherein the stem cells are induced pluripotent stem cells.

Embodiment 342. The method of any one of embodiments 324 to 341, wherein the cells are autologous cells.

Embodiment 343. The method of any one of embodiments 324 to 341, wherein the cells are allogeneic cells.

Embodiment 344. A centrifugation container comprising a centrifugation chamber with a gas-permeable silicone membrane for growing cells.

Embodiment 345. The centrifugation container of embodiment 344, wherein the gas-permeable silicone membrane is expandable.

Embodiment 346. The centrifugation container of embodiment 344 or 345, wherein the gas-permeable silicone membrane comprises a non-silicone base.

Embodiment 347. The centrifugation container of embodiment 346, wherein the non-silicone base comprises a plastic base.

Embodiment 348. The centrifugation container of embodiment 347, wherein the plastic base is a polycarbonate base or a polypropylene base.

Embodiment 349. The centrifugation container of any one of embodiments 344 to 348, wherein the centrifugation container comprises an inlet port, an outlet port, a cell pellet recovery port and a centrifugation slope.

Embodiment 350. The centrifugation container of any one of embodiments 344 to 348, wherein the centrifugation container comprises a combined inlet port and outlet port.

Embodiment 351. The centrifugation container of embodiment 349 or 350, wherein the inlet port or the combined inlet and outlet port comprises a septum.

Embodiment 352. The centrifugation container of any one of embodiments 344 to 351, wherein the cells are centrifuged in the centrifugation chamber and a cell pellet formed upon centrifugation can be recovered via the cell pellet recovery port.

Embodiment 353. A method for collecting or separating cells comprising providing cells in the centrifugation container of any one of embodiments 344 to 352 and centrifuging said cells in the centrifugation chamber.

Embodiment 354. A method for growing cells comprising providing cells in the centrifugation container of any one of embodiments 344 to 352 and culturing said cells in the centrifugation chamber.

Embodiment 355. A self-sterilizing connection comprising:
(a) a sterile inner cavity;
(b) a sterile first barrier sealing the inner cavity;
(c) a sterile needle in the inner cavity, wherein the needle comprises an inner channel; and
(d) a second barrier sealing a sterile inner lumen
   wherein the inner cavity, the first barrier and the needle are comprised in a first device, and wherein the second barrier and the inner lumen are comprised in a second device,
   wherein the second barrier is exposed to a sterilization agent, and wherein the second barrier is aligned with the first barrier and an actuation force is applied to drive the needle of the first device through both barriers to make a sterile connection with the inner lumen of the second device.

Embodiment 356. The self-sterilizing connection of embodiment 355, wherein the first device and the second device are the same device and the self-sterilizing connection makes a sterile connection within the same device.

Embodiment 357. The self-sterilizing connection of embodiment 355, wherein the first device and the second device are different and the self-sterilizing connection makes a sterile connection between different devices.

Embodiment 358. The self-sterilizing connection of embodiment 355 or 357, wherein the first device is a removable cell processing module for performing a cell processing process and the second device is a cell culture container.

Embodiment 359. The self-sterilizing connection of any one of embodiments 355 to 358, wherein the barrier is a septum.

Embodiment 360. The self-sterilizing connection of any one of embodiments 355 to 359, wherein the self-sterilizing connection is connected to a source of the sterilizing agent.

Embodiment 361. The self-sterilizing connection of any one of embodiments 355 to 360, wherein the sterilizing agent is hydrogen peroxide, isopropyl alcohol, sterile distilled water, a catalase solution, a hydrogen peroxidase solution, or a gas.

Embodiment 362. The self-sterilizing connection of embodiment 361, wherein the sterilizing agent is hydrogen peroxide.

Embodiment 363. The self-sterilizing connection of embodiment 361, wherein the sterilizing agent is isopropyl alcohol.

Embodiment 364. The self-sterilizing connection of embodiment 363, wherein the sterilizing agent is 70% isopropyl alcohol.

Embodiment 365. The self-sterilizing connection of any one of embodiments 355 to 364, wherein the actuation force is mechanical.

Embodiment 366. The self-sterilizing connection of any one of embodiments 355 to 364, wherein the actuation force is pneumatic.

Embodiment 367. The self-sterilizing connection of any one of embodiments 355 to 364, wherein the actuation force is electrical.

Embodiment 368. A method of making a sterile connection between a first device and a second device comprising:
(a) providing a self-sterilizing connection;
   wherein the self-sterilizing connection comprises:
      (i) a sterile inner cavity;
      (ii) a sterile first barrier sealing the inner cavity;
      (iii) a sterile needle in the inner cavity, wherein the needle comprises an inner channel; and
      (iv) a second barrier sealing a sterile inner lumen
   wherein the inner cavity, the first barrier and the needle are comprised in a first device, and wherein the second barrier and the inner lumen are comprised in a second device;
(b) exposing the second barrier to a sterilization agent;
(c) aligning the second barrier with the first barrier; and
(d) applying an actuation force to drive the needle of the first device through both barriers to make a sterile connection with the inner lumen of the second device.

Embodiment 369. The method of embodiment 368, wherein the first device and the second device are the same device and the self-sterilizing connection makes a sterile connection within the same device.

Embodiment 370. The method of embodiment 368, wherein the first device and the second device are different and the self-sterilizing connection makes a sterile connection between different devices.

Embodiment 371. The method of embodiment 368 or 370, wherein the first device is a removable cell processing module for performing a cell processing process and the second device is a cell culture container.

Embodiment 372. The method of any one of embodiments 368 to 371, wherein the barrier is a septum.

Embodiment 373. The method of any one of embodiments 368 to 372, wherein the self-sterilizing connection is connected to a source of the sterilizing agent.

Embodiment 374. The method of any one of embodiments 368 to 373, wherein the sterilizing agent is hydrogen peroxide, isopropyl alcohol, sterile distilled water, a catalase solution, a hydrogen peroxidase solution, or a gas.

Embodiment 375. The method of embodiment 374, wherein the sterilizing agent is hydrogen peroxide.

Embodiment 376. The method of embodiment 374, wherein the sterilizing agent is isopropyl alcohol.

Embodiment 377. The method of embodiment 374, wherein the sterilizing agent is 70% isopropyl alcohol.

Embodiment 378. The method of any one of embodiments 368 to 377, wherein the actuation force is mechanical.

Embodiment 379. The method of any one of embodiments 368 to 377, wherein the actuation force is pneumatic.

Embodiment 380. The method of any one of embodiments 368 to 377, wherein the actuation force is electrical.

Embodiment 381. A cell processing system comprising:
a cell culture container defining an internal volume, the internal volume being isolated from the ambient environment;
a cell processing module that is configured to implement a process on cells that pass through the cell processing module, the cell processing module configured to be selectively brought into and out of fluid communication with the internal volume of the cell culture container; and
a fluid handling device that is configured to drive liquid into or out of the internal volume of the cell culture container when the cell processing module is brought into fluid communication with the internal volume of the cell culture container.

Embodiment 382. The cell processing system of embodiment 381, wherein no cells, media, or reagents in the cell processing module is communicated to the fluid handling device.

Embodiment 383. The cell processing system of embodiment 381, further comprising a flow coupler including a reciprocating member and a conduit directed through the reciprocating member,
wherein the cell culture container includes a barrier,
wherein the reciprocating member of the flow coupler is advanced towards the cell cull culture container until the reciprocating member opens the barrier to bring the conduit into fluid communication with the internal volume of the cell culture container, and
wherein when the barrier is opened, the internal volume of the cell culture container is isolated from the ambient environment.

Embodiment 384. The cell processing system of embodiment 383, wherein the barrier is a septum that is coupled to the cell culture container,
wherein the reciprocating member includes a hollow tube, and
wherein when the reciprocating member is advanced towards the cell culture container, the hollow tube pierces and extends through the septum to bring the internal volume of the cell culture container into fluid communication with a flow path of the cell culture container that is isolated from the ambient environment.

Embodiment 385. The cell processing system of embodiment 384, wherein further comprising a spring that biases the reciprocating member when the reciprocating member is advanced towards the cell culture container,
wherein the spring is configured to unload to force the reciprocating member upward thereby removing the hollow tube from the internal volume of the cell culture container back through the septum, and
wherein when the hollow tube is removed back through the septum, the septum retracts to isolate the internal volume of the cell culture container from the ambient environment.

Embodiment 386. The cell processing system of embodiment 384, wherein the fluid handling device includes an actuator that is configured to extend the reciprocating member of the flow coupler towards the cell culture container.

Embodiment 387. The cell processing system of embodiment 381, wherein the cell culture container includes:
a frame having an upper piece and a lower piece;
a membrane coupled to the lower piece, the membrane defining the internal volume of the cell culture container;
a port in the upper piece of the frame; and
a septum positioned within the port that isolates the internal volume of the cell culture container from the ambient environment.

Embodiment 388. The cell processing system of embodiment 381, wherein the fluid handling device includes a pump, and
wherein at least one of:
the pump is configured to drive fluid flow from the cell processing module to the internal volume of the cell culture container, or
wherein the pump is configured to drive fluid flow from the internal volume of the cell culture container to and through the cell processing module.

Embodiment 389. The cell processing system of embodiment 381, wherein the process that the cell processing module implements on the cells is at least one of cell separation, cell collection, cell transfection, cell electroporation, cell nucleofection, cell lipofection, cell poration, cell harvesting, reagent exchange, reagent removal, or cell sampling.

Embodiment 390. The cell processing system of embodiment 381, further comprising a plurality of cell processing modules including the cell processing module, each cell processing module is configured to implement a different process on cells that flow through the cell processing module.

Embodiment 391. The cell processing system of embodiment 390, wherein only one cell processing module of the plurality of cell processing modules is configured to be fluidically connected to the cell culture container at a time.

Embodiment 392. The cell processing system of embodiment 390, wherein each cell processing module is configured to implement a single process on cells passing through the respective cell processing module.

Embodiment 393. A cell processing system comprising:
a cell culture container including a septum that isolates an internal volume of the cell culture container from the ambient environment;
a cell processing module that is configured to implement a process on cells that pass through the cell processing module, the cell processing module defining an internal flow path that is isolated from the ambient environment, the cell processing module including:
a housing; and
a flow coupler having a reciprocating member, a hollow tube coupled to the reciprocating member, a spring coupled to the reciprocating member; and
the reciprocating member is configured to be advanced towards the cell culture container until the hollow tube pierces and passes through the septum to bring a conduit of the hollow tube into fluid communication with the internal volume of the cell culture container.

Embodiment 394. The cell processing system of embodiment 393, wherein when the septum is pierced by the hollow tube, the internal volume of the cell culture container remains isolated from the ambient environment.

Embodiment 395. The cell processing system of embodiment 393, wherein the spring is configured to bias the reciprocating member when the reciprocating member is advanced towards the cell culture container, and
wherein the spring is configured to unload to force the reciprocating member upward thereby removing the hollow tube from the internal volume of the cell culture container back through the septum.

Embodiment 396. The cell processing system of embodiment 395, wherein when the hollow tube is removed back through the septum, the septum retracts to ensure that the internal volume of the cell culture container remains isolated from the ambient environment.

Embodiment 397. A method of processing cells, the method comprising:
aligning a flow coupler with a port of a cell culture container that includes a barrier, the flow coupler including a reciprocating member and a conduit that passes through the reciprocating member;
advancing the reciprocating member of the flow coupler towards the barrier of the cell culture container;
opening the barrier of the cell culture container using the reciprocating member to bring the conduit into fluid communication with an internal volume of the cell culture container; and
at least one of:
   drawing, using a fluid handling device, liquid out of the cell culture container, through the conduit of the reciprocating member, and through a cell processing module; or
   directing, using the fluid handling device, liquid from the cell processing module, through the conduit of the reciprocating member, through the port and into the internal volume of the cell culture container.

Embodiment 398. The method of embodiment 397, wherein the cell processing module is configured to implement a process on cells that pass through the cell processing module, and further comprising:
passing liquid that includes cells form the cell culture container through the cell processing module; and
performing a process on the cells that pass through the cell processing module according to the cell process associated with the cell processing module.

Embodiment 399. The method of embodiment 398, wherein the cell process is at least one of cell separation, cell collection, cell transfection, cell electroporation, cell nucleofection, cell lipofection, cell poration, cell harvesting, reagent exchange, reagent removal, or cell sampling.

Embodiment 400. The method of embodiment 399, further comprising:
placing the cell culture container into a centrifuge; and
centrifuging the cell culture container to create a cell pellet in the cell culture container.

Embodiment 401. A cell processing system comprising:
(a) a cell culture container having an interior volume configured to receive cells;
(b) a receptacle having a flow coupler with a flow path, the flow coupler being actuatable to place the flow path of the flow coupler in fluid communication with the interior volume of the cell culture container;
(c) a cell processing module defining a second flow path that is in fluid communication with the flow path of the flow coupler, the cell processing module being configured to perform one or more cell processes as cells from the interior volume of the cell culture container flow along the second flow path,
   the receptacle drawing fluid from the cell culture container, through the flow path of the flow coupler, and through the second flow path of the cell processing module, and
   the flow paths are sealed and fluidically isolated from the ambient environment surrounding the cell culture container.

### EXAMPLES

The following examples have been presented in order to further illustrate aspects of the disclosure and are not meant to limit the scope of the disclosure in any way. The examples below are intended to be examples of the present disclosure and these (and other aspects of the disclosure) are not to be bounded by theory.

### EXAMPLE 1

Some claims of the disclosure provide cell manufacturing systems and processes that provide a high-throughput, highly parallel, and flexible system. For example, in some cases, to achieve a high-throughput system, these systems focus on optimizing the utilization factor of individual subsystems, which breaks away from the idea of rigidly connected instruments and allows the cells to be moved between various instrumentation in a sterile manner. The ability to physically disconnect individual cell therapy steps and their corresponding hardware, maximizes the utility of the individual hardware components and, even importantly, maximizes the utility of whatever physical space occupied by the instrument. This concept enables physically with the advent of a standardized, closed, automation-compatible and environmentally-controllable consumable for cell culture and expansion. By separating the individual functions such as fluid handling and culture into different purpose-built instruments, this system optimizes the relative number of specific instruments to achieve maximum utilization of individual components. To accommodate different cell therapy steps, such as magnetic separation, transfection, media exchange, and sampling - just to name a few - some claims of the disclosure provide a universal instrument that is able to perform all these functions (and others). For a specific unit operation, one consumable (e.g., a cell culture container) and a second consumable (e.g., cell processing module) together in a sterile fashion inside of the fluid handling device is able to perform a type of cell processing. Various combinations of these two types of consumables allow for a closed system that is operated upon by the fluid handling device, and which allows for all of the existing cell therapy steps (and any others that will come up in the future), without modifying the instrument itself.

Some claims of the disclosure provide highly parallel and flexible design principles that allow the system to run different cell therapies in parallel. For example, the system is cell therapy/cell type agnostic with the consumables serving as conduits for cells to go between different instruments and their specific operations. This means that a single fluid handling device can perform many different cell processes iteratively, such as magnetically separating T cells, then transfecting HSCs and then feeding iPSCs. The intricacies and complexity of a given operation are condensed into standard, mass produced liquid path consumables. Furthermore, by having a standard and stand-alone consumable for cells, cell therapy research can be more easily conducted (and investigated in different ways) by allowing the scientist to mix and match individual steps. For example, if a novel cell therapy requires an additional purification (e.g., magnetic bead enabled) step, the system can easily perform this by adding another purification step with a specific liquid path.

The concept of universal instrumentation, various liquid path consumables, and standard cell consumables is scalable from a research benchtop to clinical manufacturing. To cater to both the research and clinical manufacturing fields, the system can be operated as a standalone benchtop device with staff moving the consumables between instruments, or in other configurations it can be packaged together with other automated instruments into a small format workcell. Such instruments including incubators, on-line metabolite measurement instruments, flow cytometers and others can provide various scaled cell manufacturing. Additionally, as production can be simply increased by adding additional cell processing systems.

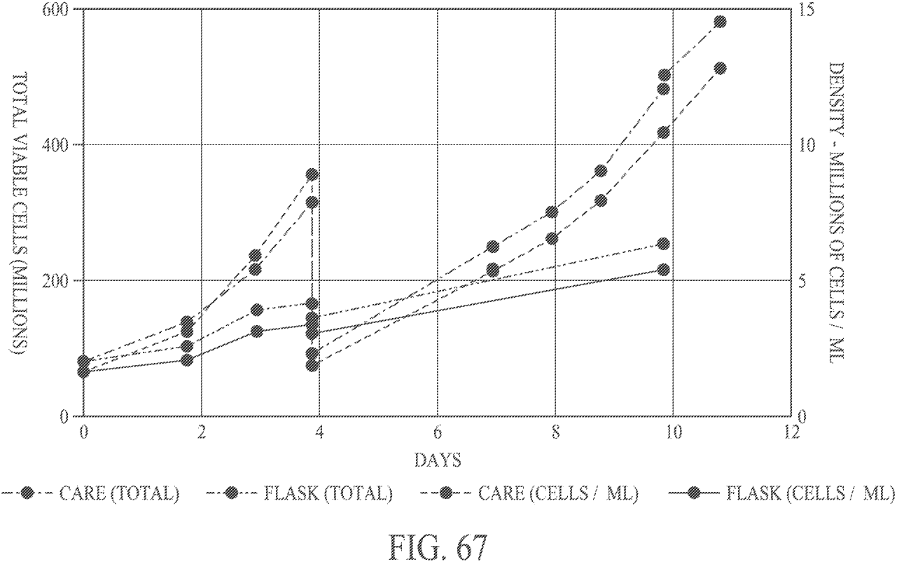

For Jurkat cell culture with cell density and viability measurements the cells were cultured in Gibco RPMI-1640 Medium ATCC modification (Fisher Scientific), supplemented with 10% heat inactivated FBS at 37° C., 5% CO₂ and 95% RH incubator. Prior to sampling the cells were resuspended in the media by gentle agitation via the sampling pipette or syringe. Cell density and viability were determined using the Nucleocounter NC-200 (Chemometec) instrument. The total number of viable cells was calculated by measuring the total volume of media and cells and multiplying it by the viability and cell concentration. During the length of the experiment to replenish nutrients and remove cell waste, the 40% of the total media in the consumables was replaced on days 2, 3, 4, 7, 8, 9 and 10 with fresh, pre-warmed culture media as indicated by circles in FIG. 67. On day 4 the cells were passaged to maintain a desirable concentration and reduce cell death.

Long term culture of Jurkat cells in large (50 ml) CARE consumables (e.g., the cell culture container described herein) were compared to culture in conventional flasks. The cells are able to sustain high cell density (12.8e6 cells/ml) conditions well in the CARE cell culture consumable with minimal loss in viability (96.4%) over the course of 11 days, reaching a total of 600 x 10⁶ cells in a single consumable. The same cells grown in a flask start show a loss in viability (93.7%) at much lower concentration of 5.38e6 cells/ml on day 10. Effectively with the CARE cell culture consumable it is possible to grow more than double the number of cells with higher viability in the same period of time as you would expect to see in a regular flask. FIG. 67 shows a graph comparing the total viable cells and density of cells for the CARE system (e.g., the cell processing system described herein) as well a standard flask.

For each experimental condition, genomic DNA (gDNA) from 10x10⁶ cells, was purified using silica-columns (Zymo Research). The gDNA was eluted in 30 uL of 1x TE pH 8.0, and quantified fluorometrically (Qubit system; Thermo Fisher Scientific). The gene editing efficiency was quantified using the QX200 Droplet Digital PCR (ddPCR) System (Bio-Rad), as follows. Briefly, 25 ng of gDNA (equivalent to approximately 7,575 copies of hgDNA), was mixed with ddPCR reagents, in a 22 uL ddPCR reaction. The reaction was compartmentalized into approximately 20,000 droplets (individual PCR microreactors) using the droplet generator (Bio-Rad). Each droplet contained DNA oligonucleotides that specifically amplifies a 379 bp amplicon of the T-cell receptor alpha constant gene of the Human genome (TRAC Gene ID: 28755; https://www.ncbi.nlm.nih.gov/gene/28755) , spanning the cut-site of the sgRNA. In addition, a pair of fluoro-labeled probes were designed to hybridize against the same PCR amplicon. Both probes were conjugated with different fluorophores at the 5' end and with a non-fluorescent quencher at the 3' end. The total number of gDNA copies in each droplet micro PCR, is proportional to the fluorescence intensity of the “reference probe” (labeled with HEX at the 5' end). The second probe called “Editing probe” (labeled with FAM at the 5' end), was designed to hybridize on top of the sgRNA cut-site and quantifies the number of gDNA copies remaining uncut after the gene editing.

Because the DNA is loaded into the droplets, following a Poisson distribution, not all the droplets contain gDNA. That allows the system to quantify gDNA at the single-copy level. Each droplet is scanned, and the fluorescent intensity of both probes is recorded, and represented in a scatter plot, for analysis. When a droplet (a single dot of the scatter plot) does not contain gDNA, it has extremely low fluorescent level. If the droplet has proportional fluorescence signal from both probes, the gDNA copy/ies inside the droplet are intact, not edited. However, if a droplet, only emits fluorescence from the “reference probe”, means that the gDNA copy/es of that given droplet were edited.

Human peripheral blood mononuclear cells (PBMCs) were isolated from the buffy coat of fresh leukopak (healthy donor; n=3) using standard Ficoll-Paque (Cytiva) density gradient centrifugation. About 30e6 fresh human PBMCs were immediately utilized in CD4+ T cell enrichment process.

Isolated PBMCs were cryopreserved in CryoStor® CS10 (STEMCELL Technologies) in CoolCell® Cell Freezing Containers (Corning) at -80° C. for 24 hours and transferred to liquid nitrogen for long term storage. Frozen human PBMCs were thawed in cold PBS / 5% FBS and DNase before CD4 positive selection as described.

To purify CD4+ T cells from human PBMCs, total cells were treated with CD4 MicroBeads (Miltenyi Biotec) at a concentration of 80 µl per 10e6 cells and incubated for 15 minutes at 4° C. degree. Cells were subsequently washed, resuspended in 1 ml buffer (DPBS, 2 mM EDTA, 0.5% FBS), and loaded into the CARE hardware platform (e.g., the cell processing system described herein) for CD4 positive selection. The recovery ratio/viability and purity post isolation were determined by Nucleocounter NC-200 (Chemometec) instrument and Flow Cytometry using CD4-PE Vio77 conjugated antibody (Miltenyi Biotec).

Anti-CD3/28 Dynabeads (Thermo Scientific) were added to the enriched CD4+ T cells, at 1:1 ratio, for cell activation and expansion. T cells were cultured in complete T cell culture media and activated for 3 - 4 days in CARE cell culture consumables. Prior to electroporation, Dynabeads were removed using the CARE hardware platform.

Activated CD4+ T cells were counted and aliquoted as 10-20e6 cells per nucleofection reaction. The sNLS-SpCas9-sNLS Nuclease was purchased from Aldevron, and the high efficiency sgRNA targeting TRAC were designed and synthesized by Synthego. For the Cas9 / Ribonucleoprotein (RNP) formation, Cas9 protein was mixed with TRAC sgRNA for 100 µl reaction. RNP complexes were then incubated with P3 buffer (Lonza) 10 minutes at room temperature.

The cells were subsequently washed and resuspended with the RNP mix. Either Lonza 4D-Nucleofector electroporation system, program EO-115 and CARE electroporator (e.g., the cell processing module described herein) were utilized to conduct TRAC locus editing. After electroporation, cells were transferred to CARE cell culture consumables.

A total of 5 conditions were tested: (1) NT: Activated CD4+ T cells incubated with P3 buffer without electroporation; (2) Mock_Lonza: Activated CD4+ T cells incubated with P3/Cas9 mixture electroporated by Lonza 4D-Nucleofector system; (3) Mock_CARE: Activated CD4+ T cells incubated with P3/Cas9 mixture electroporated by CARE electroporator; (4) KO_Lonza: Activated CD4+ T cells incubated with TRAC-RNP mix electroporated by Lonza 4D-Nucleofector system; and (5) Mock CARE: Activated CD4+ T cells incubated with TRAC-RNP mix electroporated by CARE electroporator.

All engineered cells were cultured in CARE cell culture consumables for 7 days with T cell culture media, supplemented with IL-2 and IL-15. Every 2 days, 50% of media volume was removed and replaced by fresh, pre-warmed culture media. To reach maximum expansion, cells were transferred to a larger size of CARE cell culture consumables 4 days post editing. Total cell numbers were determined by Nucleocounter NC-200 (Chemometec) at 4 and 7 days post electroporation.

Flow cytometric staining against 7-AAD (Miltenyi Biotec) was performed to assess the cell viability. The data analysis was performed using FlowJo software (FlowJo, LLC). Gating based on Forward Scatter (FSC) and Side Scatter (SSC) were used to exclude debris. Cell viability percentage was calculated as the ratio of 7-AAD negative cell number divided by the total cell number.

To determine the efficiency of CD4 positive selection, cells prior and post isolation were stained with CD4-PE Vio77 (Miltenyi Biotec). Similarly, to determine the TRAC knockout performance, cells from all five conditions mentioned above were stained with TCRalpha/beta-Violet 421 (BioLegend). Samples were acquired using MACSQuant Analyzer 10 Flow Cytometer (Miltenyi Biotec) and data were analyzed using FlowJo.

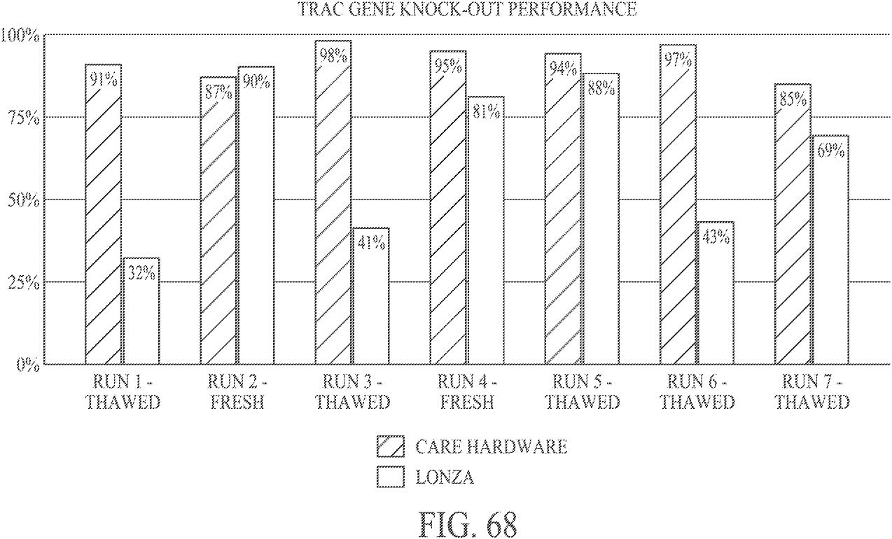

FIG. 68 shows a graph comparing a TRAC gene knock-out scores in CD4+ Primary Human T cells using CARE electroporator (e.g., the cell processing module described herein) vs Lonza’s 4D-Nucleofector electroporation system. For all conditions and experiments the CARE cell culture consumable and CARE universal liquid handler (e.g., the fluid handling device described herein) were used wherever possible. Measurements were taken on day 7 post electroporation via flow cytometry by conjugating cells with TRAC antibody labeled fluorescent protein. The CARE electroporator consistently produced high knock-out scores in both fresh and frozen primary cells.

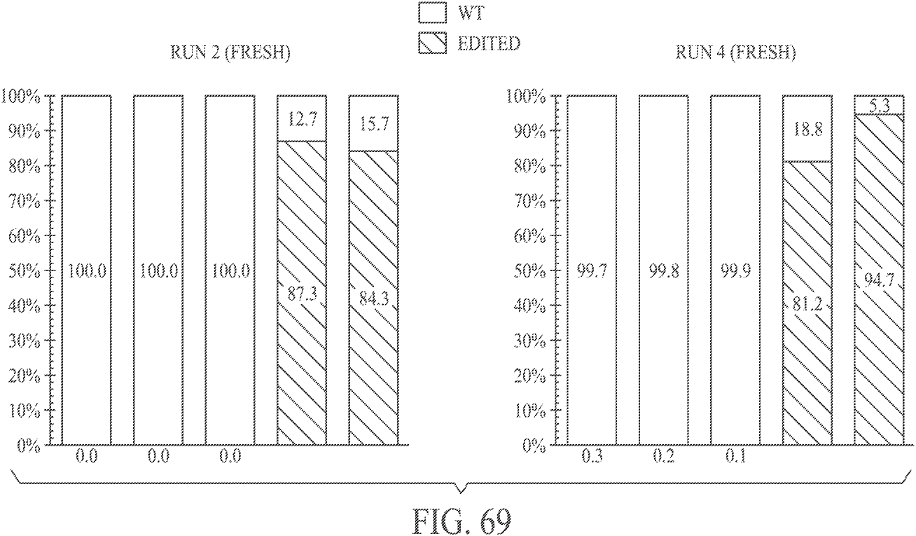

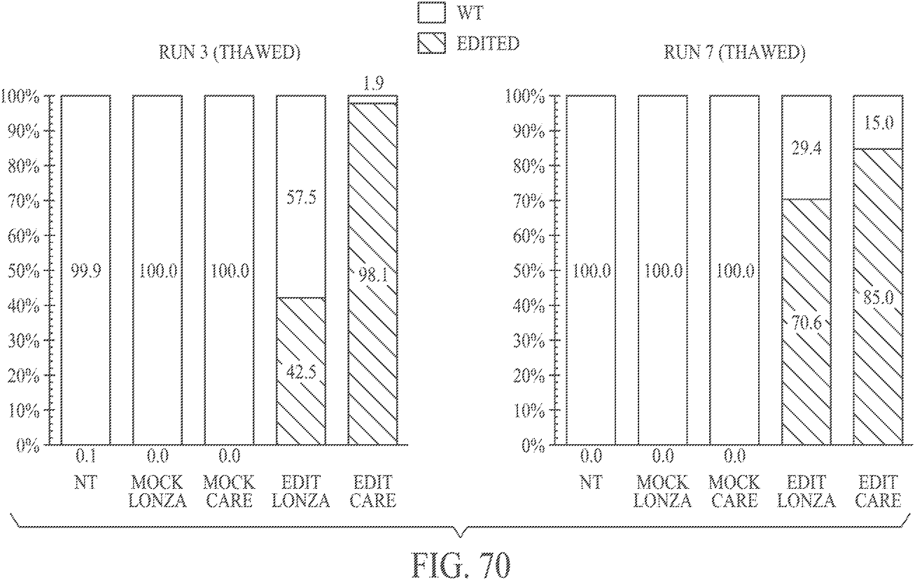

FIGS. 69 and 70 show graphs of ddPCR data for TRAC gene editing in CD4+ Human primary T cells. NT - non transfected cells; WT = non-edited; While flow cytometry analysis can quickly and effectively examine the relative presence of a cell surface protein, the data generated by droplet digital PCR provides a much more accurate analysis of gene editing at genomic level of cell populations. In theory, the genetic knock-out should lead to a loss of a cell surface protein. The ddPCR data shown here correlates very closely to the flow cytometry data, leading to a conclusion that either of the assays could be used for cell surface proteins and ddPCR alone is a good tool for confirming genomic edits. As shown in the FACS data, the CARE electroporator behaves similarly or better than Lonza’s 4D-Nucleofector.

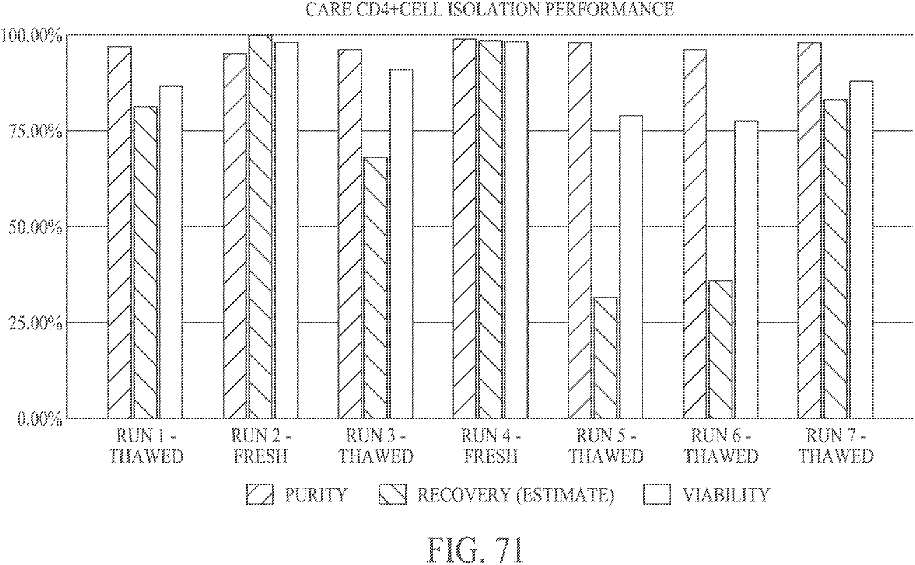

FIG. 71 shows a graph of the performance of the CARE automated hardware for magnetic isolation (e.g., the cell processing module described herein) of CD4+ T cells from fresh and thawed (from frozen) human PBMCs.

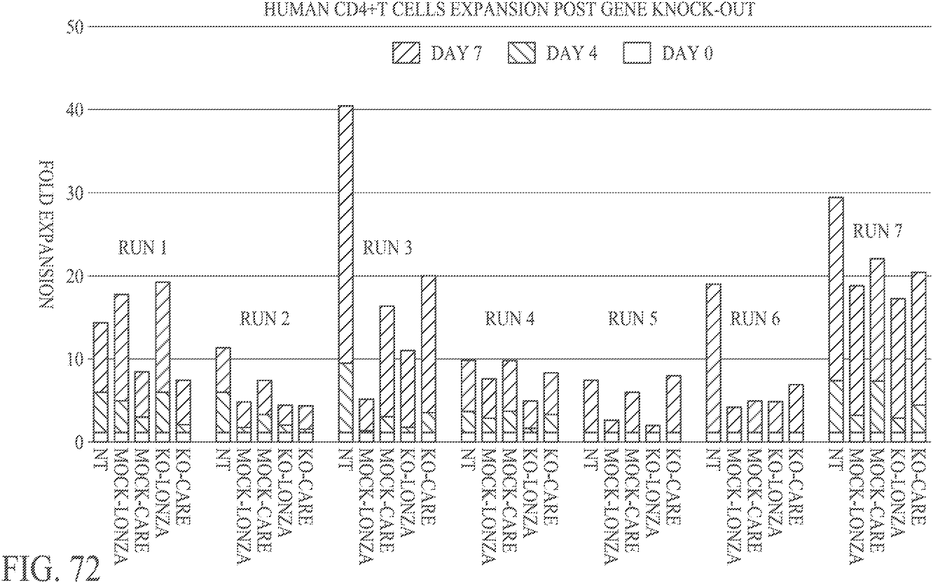

FIG. 72 shows a graph of the fold expansion of Human CD4+ T cells processed on the CARE hardware platform under 3 different conditions: NT - unedited, Mock - electroporated with Cas9 only and KO - electroporated with Cas9 and guide RNA.

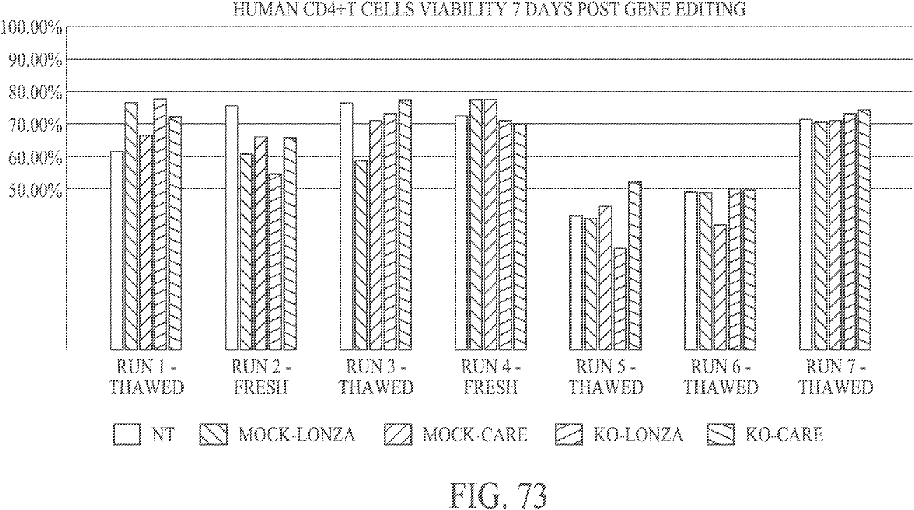

FIG. 73 shows a graph of the viability of Human CD4+ T cells isolated and culture in the CARE hardware and consumables. The cells were electroporated with Mock condition (Cas9 only) and KO condition (Cas9 + guideRNA) using the CARE electroporator and Lonza 4D Nucleofector electroporator. NT condition did not include transfection. Viability was measured by flow cytometry (7-AAD staining) 7 days post electroporation.

### EXAMPLE 2

Primary Pan T cell growth in CARE Cell Consumable - 200ml version (CCC-200) (e.g., the cell culture consumable described in FIGS. 39-42).

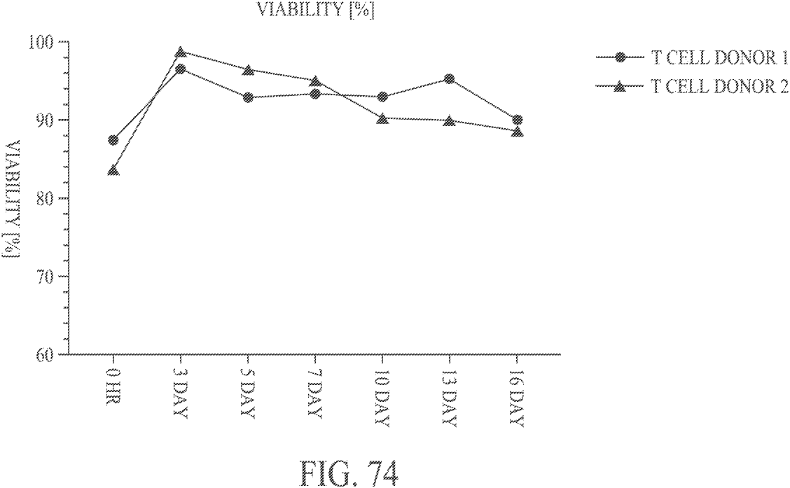

FIG. 74 shows a graph of the viability as a percentage for two independent T cell donors. Primary Pan T cells cultured in CARE Cell Consumable - 200 ml version (CCC-200) maintained high viability. The primary Pan T cells were cultured in CCC-200 for 16 days with T cell complete culture medium. The viability maintained above 85% in two independent T cell donors, and across 16 days of culture.

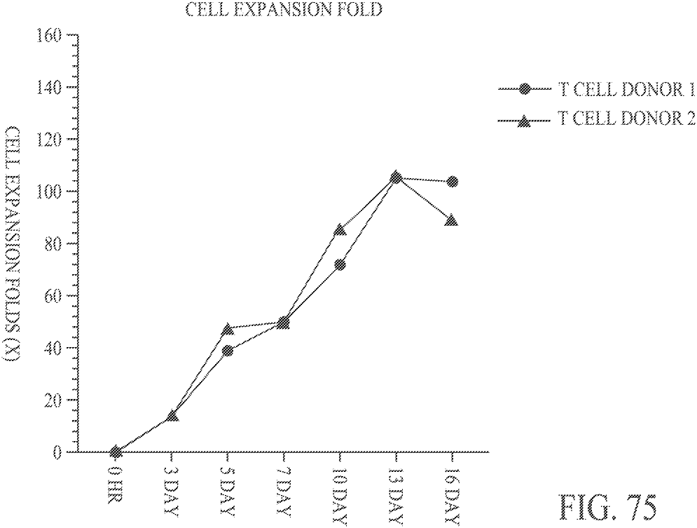

FIG. 75 shows a graph of the cell expansion folds over a number of days for the two independent T-cell donors. Primary Pan T cells expanded 100 folds and achieved more than 2.00E+009 at total viable cell number, in CCC-200. Primary Pan T cells (n=2 independent donors) were cultured in CCC-200 for 16 days with T cell complete culture medium. T cells from both donors achieved more than 100 fold expansion at 13 days of culturing, with the maximal total viable cell number greater than 2.00E+009.

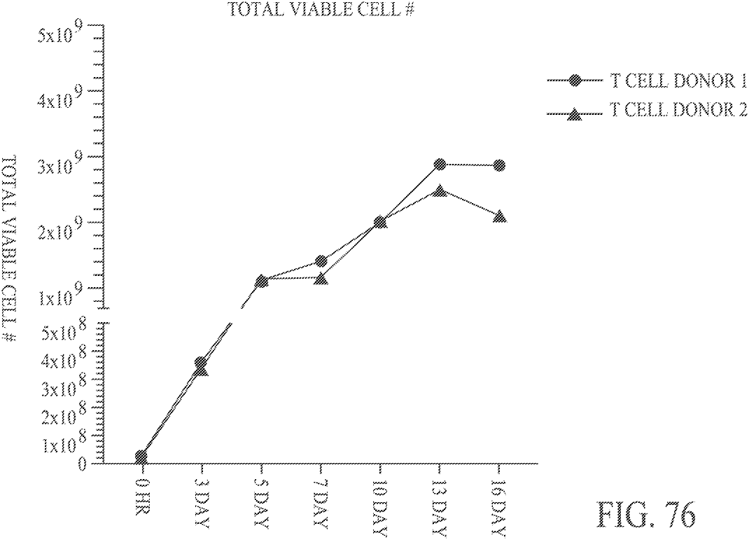

FIG. 76 shows a graph of the total number of viable cells over the number of days for the two independent T-cell donors.

Primary Pan T cell Expansion: Activated primary Pan T cells were cultured in CARE cell culture consumable (CCC) with complete T cell culture media mentioned above. The cells were seeded through CARE hardware platform, at the density within 2.3E+05 to 2.7E+05 range. Media exchange was performed every 2 - 3 days.

The present disclosure has described one or more preferred claims, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the following description or illustrated in the following drawings. The disclosure is capable of other claims and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular claims or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or claims. Further, references to particular rotational or other movements (e.g., counterclockwise rotation) is generally intended as a description only of movement relative a reference frame of a particular example of illustration.

In some claims, aspects of the disclosure, including computerized configurations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, claims of the disclosure can be implemented as a set of instructions, tangibly embodied on a non-transitory computer-readable media, such that a processor device can implement the instructions based upon reading the instructions from the computer-readable media. Some claims of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for configuration of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular claims of the disclosure. Further, in some claims, certain operations can be executed in parallel, including by dedicated parallel processing devices, or separate computing devices configured to interoperate as part of a large system.

As used herein in the context of computer configuration, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some configurations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as claims of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” For example, a list of “one of A, B, or C” indicates options of: A, but not B and C; B, but not A and C; and C, but not A and B. A list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of A, one or more of B, and one or more of C. Similarly, a list preceded by "a plurality of' (and variations thereon) and including "or" to separate listed elements indicates options of multiple instances of any or all of the listed elements. For example, the phrases “a plurality of A, B, or C” and “two or more of A, B, or C” indicate options of: A and B; B and C; A and C; and A, B, and C.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or 'B or “A and B.”

This discussion is presented to enable a person skilled in the art to make and use claims of the disclosure. Various modifications to the illustrated examples will be readily apparent to those skilled in the art, and the generic principles herein can be applied to other examples and applications without departing from the principles disclosed herein. Thus, claims of the disclosure are not intended to be limited to claims shown, but are to be accorded the widest scope consistent with the principles and features disclosed herein and the claims below. The following detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Various features and advantages of the disclosure are set forth in the following claims.

1-381. (canceled)

382. A cell processing system comprising:
a cell culture container defining an internal volume, the internal volume being isolated from the ambient environment;
a cell processing module that is configured to implement a process on cells that pass through the cell processing module, the cell processing module configured to be selectively brought into and out of fluid communication with the internal volume of the cell culture container; and
a fluid handling device that is configured to drive liquid into or out of the internal volume of the cell culture container when the cell processing module is brought into fluid communication with the internal volume of the cell culture container.

383. The cell processing system of claim 382, wherein no cells, media, or reagents in the cell processing module is communicated to the fluid handling device.

384. The cell processing system of claim 382, further comprising a flow coupler including a reciprocating member and a conduit directed through the reciprocating member,
wherein the cell culture container includes a barrier,
wherein the reciprocating member of the flow coupler is advanced towards the cell culture container until the reciprocating member opens the barrier to bring the conduit into fluid communication with the internal volume of the cell culture container, and
wherein when the barrier is opened, the internal volume of the cell culture container is isolated from the ambient environment.

385. The cell processing system of claim 384, wherein the barrier is a septum that is coupled to the cell culture container,
wherein the reciprocating member includes a hollow tube, and
wherein when the reciprocating member is advanced towards the cell culture container, the hollow tube pierces and extends through the septum to bring the internal volume of the cell culture container into fluid communication with a flow path of the cell culture container that is isolated from the ambient environment.

386. The cell processing system of claim 385, wherein further comprising a spring that biases the reciprocating member when the reciprocating member is advanced towards the cell culture container,
wherein the spring is configured to unload to force the reciprocating member upward thereby removing the hollow tube from the internal volume of the cell culture container back through the septum, and
wherein when the hollow tube is removed back through the septum, the septum retracts to isolate the internal volume of the cell culture container from the ambient environment.

387. The cell processing system of claim 385, wherein the fluid handling device includes an actuator that is configured to extend the reciprocating member of the flow coupler towards the cell culture container.

388. The cell processing system of claim 382, wherein the cell culture container includes:
a frame having an upper piece and a lower piece;
a membrane coupled to the lower piece, the membrane defining the internal volume of the cell culture container;
a port in the upper piece of the frame; and
a septum positioned within the port that isolates the internal volume of the cell culture container from the ambient environment.

389. The cell processing system of claim 382, wherein the fluid handling device includes a pump, and
wherein at least one of:
the pump is configured to drive fluid flow from the cell processing module to the internal volume of the cell culture container, or
wherein the pump is configured to drive fluid flow from the internal volume of the cell culture container to and through the cell processing module.

390. The cell processing system of claim 382, wherein the fluid handling device includes a pressure source for delivering at least one of positive pressure or negative pressure, and
wherein at least one of:
the pressure source is configured to drive fluid flow from the cell processing module to the internal volume of the cell culture container, or
wherein the pressure source is configured to drive fluid flow from the internal volume of the cell culture container to and through the cell processing module.

391. The cell processing system of claim 382, wherein the process that the cell processing module implements on the cells is at least one of cell separation, cell collection, cell transfection, cell electroporation, cell nucleofection, cell lipofection, cell poration, cell harvesting, reagent exchange, reagent removal, or cell sampling.

392. The cell processing system of claim 382, further comprising a plurality of cell processing modules including the cell processing module, each cell processing module is configured to implement a different process on cells that flow through the cell processing module.

393. The cell processing system of claim 392, wherein only one cell processing module of the plurality of cell processing modules is configured to be fluidically connected to the cell culture container at a time.

394. The cell processing system of claim 392, wherein each cell processing module is configured to implement a single process on cells passing through the respective cell processing module.

395. A cell processing system comprising:
a cell culture container including a septum that isolates an internal volume of the cell culture container from the ambient environment;
a cell processing module that is configured to implement a process on cells that pass through the cell processing module, the cell processing module defining an internal flow path that is isolated from the ambient environment, the cell processing module including:
a housing; and
a flow coupler having a reciprocating member, a hollow tube coupled to the reciprocating member, a spring coupled to the reciprocating member; and
the reciprocating member is configured to be advanced towards the cell culture container until the hollow tube pierces and passes through the septum to bring a conduit of the hollow tube into fluid communication with the internal volume of the cell culture container.

396. The cell processing system of claim 395, wherein when the septum is pierced by the hollow tube, the internal volume of the cell culture container remains isolated from the ambient environment.

397. The cell processing system of claim 395, wherein the spring is configured to bias the reciprocating member when the reciprocating member is advanced towards the cell culture container, and
wherein the spring is configured to unload to force the reciprocating member upward thereby removing the hollow tube from the internal volume of the cell culture container back through the septum.

398. The cell processing system of claim 397, wherein when the hollow tube is removed back through the septum, the septum retracts to ensure that the internal volume of the cell culture container remains isolated from the ambient environment.

399. A method of processing cells, the method comprising:
aligning a flow coupler with a port of a cell culture container that includes a barrier, the flow coupler including a reciprocating member and a conduit that passes through the reciprocating member;
advancing the reciprocating member of the flow coupler towards the barrier of the cell culture container;
opening the barrier of the cell culture container using the reciprocating member to bring the conduit into fluid communication with an internal volume of the cell culture container; and at least one of:
drawing, using a fluid handling device, liquid out of the cell culture container, through the conduit of the reciprocating member, and through a cell processing module; or
directing, using the fluid handling device, liquid from the cell processing module, through the conduit of the reciprocating member, through the port and into the internal volume of the cell culture container.

400. The method of claim 399, wherein the cell processing module is configured to implement a process on cells that pass through the cell processing module, and further comprising:
passing liquid that includes cells form the cell culture container through the cell processing module; and
performing a process on the cells that pass through the cell processing module according to the cell process associated with the cell processing module.

401. The method of claim 400, wherein the cell process is at least one of cell separation, cell collection, cell transfection, cell electroporation, cell nucleofection, cell lipofection, cell poration, cell harvesting, reagent exchange, reagent removal, or cell sampling.

402. The method of claim 401, further comprising:
placing the cell culture container into a centrifuge; and
centrifuging the cell culture container to create a cell pellet in the cell culture container.
